(19) United States
(12) Patent Application Publication
(10) Pub. No.: US 2014/0088052 A1
(43) Pub. Date: Mar. 27, 2014

(54) CHALCONE DERIVATIVES AS NRF2 ACTIVATORS

(71) Applicants: Shyam Biswal, Ellicott City, MD (US); Rajesh Thimmulappa, Baltimore, MD (US); Sarvesh Kumar, Lutherville-timonium, MD (US); Sanjay V. Malhotra, Rockville, MD (US); Vineet Kumar, Rockville, MD (US); Kim Jung-Hyun, Baltimore, MD (US)

(72) Inventors: Shyam Biswal, Ellicott City, MD (US); Rajesh Thimmulappa, Baltimore, MD (US); Sarvesh Kumar, Lutherville-timonium, MD (US); Sanjay V. Malhotra, Rockville, MD (US); Vineet Kumar, Rockville, MD (US); Kim Jung-Hyun, Baltimore, MD (US)

(73) Assignee: THE JOHNS HOPKINS UNIVERSITY, Baltimore, MD (US)

(21) Appl. No.: 14/000,816

(22) PCT Filed: Feb. 27, 2012
(86) PCT No.: PCT/US2012/026744

§ 371 Date: Dec. 10, 2013

Related U.S. Application Data
(60) Provisional application No. 61/446,716, filed on Feb. 25, 2011.
(60) Provisional application No. 61/500,272, filed on Jun. 23, 2011.

(51) Int. Cl. A61K 31/121; A61K 45/06; C07C 49/84

(52) U.S. Cl. CPC A61K 31/121; C07C 49/84; A61K 45/06
USPC 514/171; 568/334; 514/685

(57) ABSTRACT

Compounds and methods for treating or preventing a disease, disorder or condition associated with an Nrf2-regulated pathway, including those associated with an autoimmune disease, comorbidity associated with diabetes, such as retinopathy and nephropathy, bone marrow transplant for leukemia and related cancers, bone marrow deficiencies, inborn errors of metabolism, and other immune disorders, oxidative stress, respiratory infection, ischemia, neurodegenerative disorders, radiation injury, neutropenia caused by chemotherapy, autoimmunity, and congenital neutropenic disorders, and for restoring a corticosteroid responsiveness, in a subject are provided.

### CROSS-REFERENCE TO RELATED APPLICATIONS

This application claims the benefit of U.S. Provisional Application Nos. 61/446,716, filed Feb. 25, 2011, and 61/500,272, filed Jun. 23, 2011, each of which is incorporated herein by reference in its entirety.

### FEDERALLY SPONSORED RESEARCH OR DEVELOPMENT

This invention was made with government support under HL081205 and AI080541 awarded by the National Institutes of Health. The government has certain rights in the invention.

### BACKGROUND

Nuclear factor erythroid-2 related factor 2 (Nrf2) is a basic leucine zipper transcription factor, which regulates a transcriptional program that maintains cellular redox homeostasis and protects cells from oxidative insult. Nrf2 activates transcription of its target genes through binding specifically to the antioxidant-response element (ARE) found in those gene promoters. The Nrf2-regulated transcriptional program includes a broad spectrum of genes, including antioxidants, such as γ-glutamyl cysteine synthetase modifier subunit (GCLm), γ-glutamyl cysteine synthetase catalytic subunit (GCLc), heme oxygenase-1, superoxide dismutase, glutathione reductase (GSR), glutathione peroxidase, thioredoxin, thioredoxin reductase, peroxiredoxins (PRDX), cysteine/glutamate transporter (SLC7A11), phase II detoxification enzymes NADP(H) quinone oxidoreductase 1 (NQO1), GST, UDP-glucuronosyltransferase, and several ATP-dependent drug efflux pumps, including MRP1 and MRP2.

Nrf2 protects cells and multiple tissues by coordinately up-regulating ARE-related detoxification and antioxidant genes and molecules required for the defense system. Nrf2-activation suppresses oxidative stress and inflammation and has been shown to be neuroprotective. Accordingly, therapeutic strategies that increase Nrf2 biological activity or expression can be used to treat or prevent diseases, disorders, or conditions related to oxidative stress, including inflammatory disorders, and neurodegenerative disorders.

### SUMMARY

In some aspects, the presently disclosed subject matter provides compounds and methods for treating or preventing a disease, disorder or condition associated with an Nrf2-regulated pathway, including those associated with an autoimmune disease, comorbidity associated with diabetes, such as retinopathy and nephropathy, bone marrow transplant for leukemia and related cancers, bone marrow deficiencies, inborn errors of metabolism, and other immune disorders, oxidative stress, respiratory infection, ischemia, neurodegenerative disorders, radiation injury, chemotherapy injury, neutropenia caused by chemotherapy, autoimmunity, and congenital neutropenic disorders, and for restoring a corticosteroid responsiveness.

In particular aspects, the presently disclosed subject matter provides a compound of Formula (Ia):

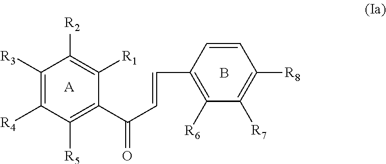

wherein R₁, R₂, R₃, R₄, and R₅ are each independently selected from the group consisting of H and alkoxyl, provided that at least one of R₁, R₂, R₃, R₄, and R₅ is alkoxyl; R₆ and R₇, R₈ are each independently selected from the group consisting of H, CF₃, and NO₂, provided that at least one of R₆ and R₇ is CF₃ or NO₂; and R₈ is H; under the further provision that if R₆ or R₇ is CF₃, then R₁ and R₃, or R₂ and R₃, or R₁ and R₄ cannot both be alkoxyl; and pharmaceutically acceptable salts thereof.

In other aspects, the presently disclosed subject matter provides a method for treating or preventing a disease, disorder or condition associated with an Nrf2-regulated pathway, the method comprising administering a compound of Formula (Ib) to the subject in an amount effective to increase an Nrf2 biological activity or Nrf2 expression, thereby treating or preventing the disease, disorder, or condition:

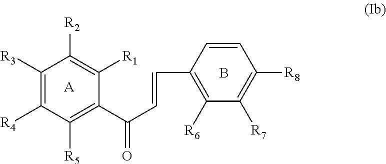

wherein: R₁, R₂, R₃, R₄, and R₅ are each independently selected from the group consisting of H and alkoxyl, provided that at least one of R₁, R₂, R₃, R₄, and R₅ is alkoxyl; R₆, R₇, and R₈ are each independently selected from the group consisting of H, CF₃, and NO₂, provided that at least one of R₆, R₇, and R₈ is CF₃ or NO₂; and pharmaceutically acceptable salts thereof.

In some aspects, the disease, disorder, or condition is an autoimmune disease. In particular aspects, the autoimmune disease is selected from the group consisting of acute graft-versus host disease, autoimmune inner ear disease, inflammatory bowel disease, rheumatoid arthritis, psoriasis, psoriatic arthritis, multiple sclerosis, scleroderma, lupus, ankylosing spondylitis, neutropenia, and uveitis.

In other aspects, the presently disclosed subject matter provides compositions and methods for treating or preventing a comorbidity associated with diabetes including, but not limited to, retinopathy and nephropathy.

In yet other aspects, the presently disclosed subject matter provides compositions and methods for improving the outcome for bone marrow transplant for leukemia and related cancers and treating bone marrow deficiencies, inborn errors of metabolism, and immune disorders.

In certain aspects, the disease, disorder, or condition is related to oxidative stress, for example, a pulmonary inflammatory condition, pulmonary fibrosis, asthma, chronic obstructive pulmonary disease (COPD), emphysema, sepsis, septic shock, meningitis, encephalitis, hemorrhage, ischemic injury, cerebral ischemia, heart ischemia, a cognitive deficit, and a neurodegenerative disorder.

In further aspects, the presently disclosed method restores a corticosteroid responsiveness in the subject, for example, in a subject that has or is at risk of developing a disease, disorder, or condition selected from the group consisting of chronic obstructive pulmonary disease (COPD), asthma, severe asthma, acute graft-versus host disease, autoimmune inner ear disease, inflammatory bowel disease, and rheumatoid arthritis.

In yet other aspects, the disease, disorder, or condition comprises a respiratory infection, for example, in a subject that has or is at risk of developing a disease, disorder, or condition selected from the group consisting of an acute respiratory infection, chronic bronchitis, cystic fibrosis, and an immunodeficiency syndrome.

In yet further aspects, the presently disclosed method includes treating or preventing a radiation injury in the subject, for example, a radiation injury arising as a result of radiotherapy, accidental radiation exposure, or nuclear attack.

In some aspects, the presently disclosed subject matter provides a method for treating of preventing neutropenia caused by chemotherapy, autoimmunity diseases, and in subjects having a congenital neutropenic disorder.

In other aspects, the presently disclosed subject matter provides a kit for treating or preventing radiation injury, the kit comprising a therapeutically effect amount of compound of Formula (Ia) and written instructions for use of the kit.

In yet other aspects, the presently disclosed subject matter provides a device for dispersing one or more particles comprising a compound of Formula (Ia) in an amount effective to increase a Nrf2 biological activity or Nrf2 expression and delivering a dose of the particles to lung tissue of a subject. In some aspects, the device can be a nebulizer, a metered dose inhaler, or a dry powder inhaler.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE FIGURES

Having thus described the presently disclosed subject matter in general terms, reference will now be made to the accompanying Figures, which are not necessarily drawn to scale, and wherein:

FIG. 1 shows the structure activity relationship of chalcone derivatives;

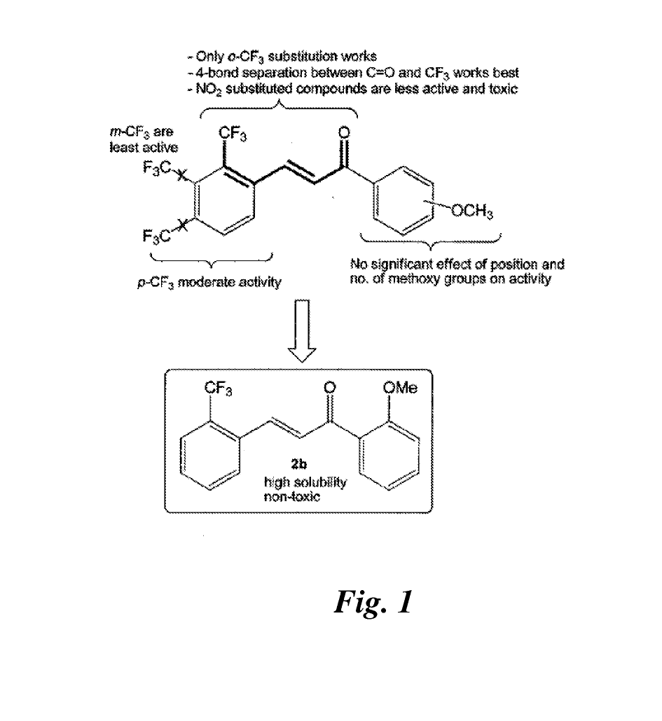

FIG. 2 shows the expression of Nrf2-regulated genes in small intestine after treatment with the presently disclosed chalcone derivatives. Mice (n=4) were fed with vehicle (DCP-10% DMSO+10% Cremophor+80% phosphate buffered saline) or chalcone derivatives or sulforaphane (50 mg/kg body weight) by gavage, and the small intestines were harvested 24 hours later. The expression of Nrf2-regulated genes GCLM and NQO1 was analyzed in the tissues by qRT-PCR as a surrogate marker of Nrf2 activity. β-actin was used for normalization. Data are representative of three independent experiments. Values shown are mean±SD of triplicate wells (P≦0.05);

FIG. 3 shows levels of NQO1-ARE luciferase activity after treatment with compound 2a. NQO1-ARE luciferase activity was measured by using stably transfected Beas-2B cells after treatment with compound 2b or sulforaphane (SFN) or dimethyl sulfoxide (DMSO). The exposure to compound 2b resulted in a significant concentration-dependent increase in luciferase activity as relative luminescence intensity (RLI). Data are representative of three independent experiments. Values shown are mean±SD of triplicate wells (P≦0.05);

FIG. 4 shows the expression of Nrf2-regulated genes after treatment with compound 2b. Human bronchial epithelial cells (Beas-2B) were treated with compound 2b at the indicated concentrations for 16-20 hours. The expression of Nrf2-regulated genes GCLM, HO1, and NQO1 was analyzed in the tissues by qRT-PCT as a surrogate marker of Nrf2 activity. β-actin was used for normalization. Data are representative of three independent experiments. Values shown are mean±SD of triplicate wells (P≦0.05);

FIG. 5 shows a time-dependent increase in Nrf2-regulated genes after treatment with compound 2b. Human bronchial epithelial cells (Beas-2B) were treated with compound 2b (10 μM) at various time points. The expression of Nrf2-regulated genes GCLM, HO1, and NQO1 was analyzed in the tissues by aRT-PCR. β-actin was used for normalization. Data are representative of three independent experiments. Values shown are mean±SD of triplicate wells (P≦0.05);

FIG. 6 demonstrates that activation of Nrf2 genes by compound 2b is independent of reactive oxygen species (ROS) generation. Human bronchial epithelial cells (Beas-2B) were treated with compound 2b (10 μM) in the presence of an antioxidant, N-acetyl cysteine (10 mM, NAC). Cells were harvested 24 h after the treatment and the Nrf2-driven expression of NQO1, HO-1, and GCLM was quantified. β-actin was used for normalization. Data are representative of three independent experiments. Values shown are mean±SD of triplicate wells (P≦0.05);

FIG. 7 shows survival rates of mice treated with compound 2b one hour after total body irradiation (7.16 Gy). Five additional doses of compound 2b or vehicle were given every 48 hours after the first dose. Mortality was monitored for 30 days. (n=10 mice/gp);

FIGS. 8A and 8B show (A) a pictorial version of the regulation of Nrf2 in the cell and (B) the role of Nrf2 in upregulating other genes (Prior Art);

FIGS. 9A and 9B show the percent survival of mice after total body irradiation with a dose of either 6.9 Gy (A) and 7.1 Gy (B) and administration of either compound 2b or vehicle (PEG-200) at 24 h after irradiation;

FIG. 10 shows the percent survival of mice after total body irradiation with a dose of 7.3 Gy and administration of either compound 2b or vehicle at 1 h, 6 h, or 24 h after irradiation;

FIGS. 11A-11C show hematopoietic recovery after total body irradiation with a dose of 6.9 Gy and administration of either compound 2b or vehicle at 24 h after irradiation. Hematopoietic recovery is shown by (A) longitudinal analysis of white blood cells, neutrophils, and lymphocytes, (B) total white blood cell count, red blood cell count, and platelet count on day 22 after irradiation, and (C) histopathological analysis by H&E staining of bone marrow cellularity on day 7 and day 20 after irradiation;

FIGS. 12A-12C show an analysis of the markers of Nrf2 activity (NQO1, HO-1, and GCLM) in bone marrow mononuclear cells (A), lung tissue (B), and small intestine tissue (C) after total body irradiation with a dose of 6.9 Gy and administration of either compound 2b or vehicle;

FIGS. 13A-13C show (A) the levels of total bone marrow mononuclear cells, (B) the frequency of subpopulation of hematopoietic stem cells by FACS analysis; and (C) the total number of viable hematopoietic stem cells after total body irradiation with a dose of 6.9 Gy and administration of either compound 2b or vehicle;

FIG. 14 shows the effect of compound 2b on mice with induced reversible neutropenia. Neutrophils were analyzed in the peripheral blood at the indicated time periods;

FIGS. 15A and 15B show the effect of compound 2b on mice with induced autoimmune encephalomyelitis. Mice were assessed for prophylactic efficacy of compound 2b (A) and therapeutic efficacy (B);

FIGS. 16A and 16B show the effect of compound 2b on mice with induced asthma. Mice were assessed for airway inflammation (A) and airway hyperresponsiveness (B);

FIG. 17 shows the effect of compound 2b on the ability of mouse macrophages to clear bacteria; and

FIG. 18 shows the effect of compound 2b on the ability of mouse macrophages to inhibit lipopolysaccharide-induced inflammation.

### DETAILED DESCRIPTION

The presently disclosed subject matter now will be described more fully hereinafter with reference to the accompanying Figures, in which some, but not all embodiments of the presently disclosed subject matter are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

Nuclear factor-erythroid 2 p45-related factor 2 (Nrf2) plays a central role in protecting cells from oxidative stress and inflammation by increasing several cytoprotective pathways. Such cytoprotective pathways include antioxidant enzymes, which scavenge and decompose free radicals, Phase II enzymes, which detoxify electrophiles, and the proteasome system, which removes damaged proteins. Nrf2-deficient mice are more sensitive to oxidative stress and show an increase in susceptibility and severity to several inflammatory disorders, including chronic obstructive pulmonary disease (COPD), asthma, radiation-induced normal tissue injuries, and neurodegenerative diseases. Activation of Nrf2 protects mice from these and related disorders by suppressing oxidative stress and inflammation. Accordingly, Nrf2 is a potential drug target for treating disorders related to oxidative stress and from autoimmune diseases by suppressing inflammation.

In some embodiments, the presently disclosed subject matter provides chalcone derivatives that activate Nrf2 and increases antioxidant and anti-inflammatory defenses in mouse tissues. As provided in more detail herein below, the presently disclosed compounds can be used for treating or preventing diseases, disorders, or conditions associated with Nrf2-regulated pathways, including, but not limited to an autoimmune disease, comorbidity associated with diabetes, such as retinopathy and nephropathy, bone marrow transplant for leukemia and related cancers, bone marrow deficiencies, inborn errors of metabolism, and other immune disorders, oxidative stress, respiratory infection, ischemia, neurodegenerative disorders, radiation injury, neutropenia caused by chemotherapy, autoimmunity, and congenital neutropenic disorders, and for restoring a corticosteroid responsiveness.

### I. Chalcone Derivatives as Nrf2 Activators

Nrf2-mediated activation of antioxidant response element (ARE) is a central part of molecular mechanisms governing the protective function of phase II detoxification and antioxidant enzymes against oxidative stress and inflammation. By “Nrf2 polypeptide” is meant a protein or protein variant, or fragment thereof, that comprises an amino acid sequence substantially identical to at least a portion of GenBank Accession No. NPJ306164 (human nuclear factor (erythroid-derived 2)-like 2) and that has an Nrf2 biological activity (e.g., activation of target genes through binding to antioxidant response element (ARE), regulation of expression of antioxidants and xenobiotic metabolism genes).

Nrf2 is sequestered in the cytoplasm by its repressor, Keap. Modification of cysteine residues in Keap1 by a variety of inducers, specifically Michael acceptors, results in a conformational change that renders Keap1 to dissociate from Nrf2, thereby inducing translocation of Nrf2 to the nucleus. By “Keap1 polypeptide” is meant a polypeptide comprising an amino acid sequence having at least 85% identity to GenBank Accession No. AAH21957. By “Keap1 nucleic acid molecule” is meant a nucleic acid molecule that encodes a Keap1 polypeptide or fragment thereof.

Representative Nrf2-regulated gene functions are summarized in Table 1.

TABLE 1
NRF2-regulated Gene Functions

| Target Genes | Functions |
|---|---|
| Heme oxygenase-1, Ferritin, | Direct antioxidants |
| NQO1, SOD1 | |
| GCLM, GCLC, GCS, GSR | Increase the levels of GSH synthesis and regeneration |
| G6PD, malic enzyme | Stimulate NADPH synthesis |
| GSTs, UGTs | Encode enzymes that directly inactivate oxidants or |
| | electrophiles |
| GPX2, peroxiredoxin | Increases detoxification of H₂O₂, peroxynitrite, and |
| | oxidative damage by products (4HNE, lipid |
| | hydroperoxides); |
| | Enhance the recognition and repair and removal of |
| | damaged DNA |
| Heat shock proteins (HSP | Chaperone activity; Enhance the recognition, repair, |
| 70), Proteosome members | and removal of damaged proteins |
| MRP1 | Enhance toxin export via the multidrug response |
| | transporters |
| Leukotriene B4 12- | Inhibits cytokine mediated inflammation |
| hydroxydehydrogenase | |
| CD36, MARCO (scavenger | i) Enhances phagocytosis of bacteria |
| receptors) | ii) Maintenance of tissue homeostasis and resolution |
| | of inflammatory lesions by clearance of apoptotic |
| | cells |
| Suppress NF-KB signaling | Regulates redox dependent innate immune, as well as |
| | adaptive immune response |

Chalcones, i.e., 1,2-diphenyl-2-propen-1-ones, are Michael acceptors reported to possess a wide variety of biological properties. The presently disclosed subject matter discloses the synthesis of a series of chalcone derivatives, which were tested for their Nrf2 activity in human bronchial epithelial cells. Eight chalcone derivatives were determined to exhibit positive Nrf2 activity and were further tested in a mice model. Of these eight chalcones, 2-trifluoromethyl-2′-methoxychalone (2b) emerged as a potent activator of Nrf2 in mice. Further, a quantitative structure-activity relationship is disclosed and a possible mechanism of Nrf2 activation is provided.

A. Compounds of Formula (Ia)

In some embodiments, the presently disclosed subject matter provides a compound of Formula (Ia):

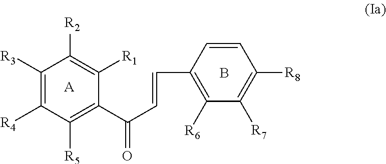

wherein: R₁, R₂, R₃, R₄, and R₅ are each independently selected from the group consisting of H and alkoxyl, provided that at least one of R₁, R₂, R₃, R₄, and R₅ is alkoxyl; R₆ and R₇, R₈ are each independently selected from the group consisting of H, CF₃, and NO₂, provided that at least one of R₆ and R₇ is CF₃ or NO₂; and R₈ is H; under the further provision that if R₆ or R₇ is CF₃, then R₁ and R₃, or R₂ and R₃, or R₁ and R₄ cannot both be alkoxyl; and pharmaceutically acceptable salts thereof.

Methods of making compounds of Formula (Ia) are provided in Example 1, herein below. Representative compounds of Formula (Ia) are provided in Table 2. It should be noted that compounds of Formula (Ia) do not include compounds 2e, 2g, 2h, 3g, and 3h of Table 2.

When the term “independently selected” is used, the substituents being referred to (e.g., R groups, such as groups R₁, R₂, and the like, or variables, such as “m” and “n”), can be identical or different, unless specified otherwise herein. For example, both R₁ and R₂ can be substituted alkyls, or R₁ can be hydrogen and R₂ can be a substituted alkyl, and the like.

The terms “alkoxyl” or “alkoxy” are used interchangeably herein and refer to a saturated (i.e., alkyl-O—) or unsaturated (i.e., alkenyl-O— and alkynyl-O—) group attached to the parent molecular moiety through an oxygen atom, and, in some embodiments, can include C₁₋₂₀ inclusive, linear, branched, or cyclic, saturated or unsaturated oxo-hydrocarbon chains, including, for example, methoxyl, ethoxyl, propoxyl, isopropoxyl, n-butoxyl, sec-butoxyl, t-butoxyl, and n-pentoxyl, neopentoxy, n-hexoxy, and the like.

The terms “halo,” “halide,” or “halogen” as used herein refer to fluoro, chloro, bromo, and iodo groups. Additionally, terms, such as “haloalkyl,” are meant to include monohaloalkyl and polyhaloalkyl. For example, the term “halo(C₁-C₄)alkyl” is mean to include, but not be limited to, trifluoromethyl; 2,2,2-trifluoroethyl; 4-chlorobutyl; 3-bromopropyl; and the like.

The term “nitro” refers to the —NO₂ group.

In some embodiments, the compound of Formula (Ia) is selected from the group consisting of:

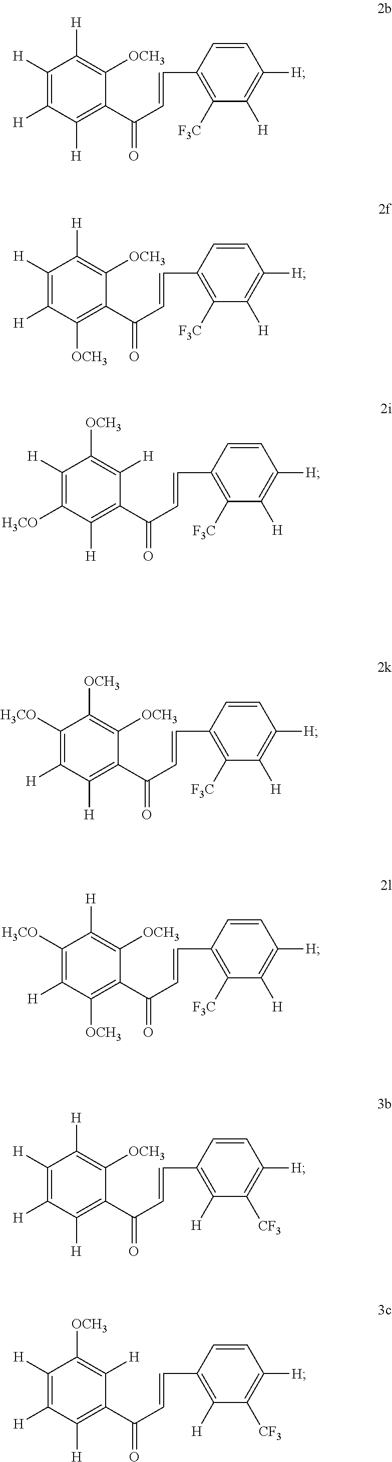

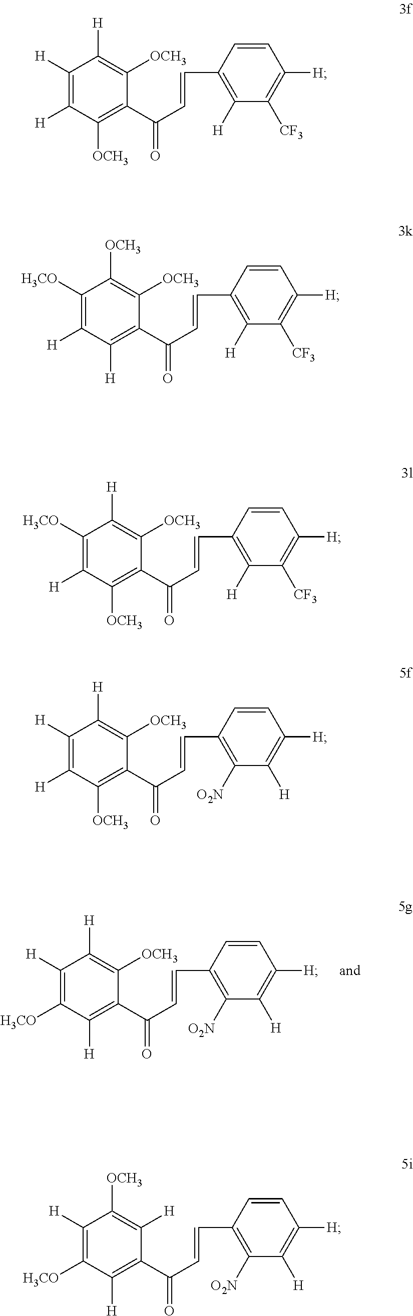

and pharmaceutically acceptable salts thereof.

In other embodiments, the compound of Formula (Ia) is selected from the group consisting of:

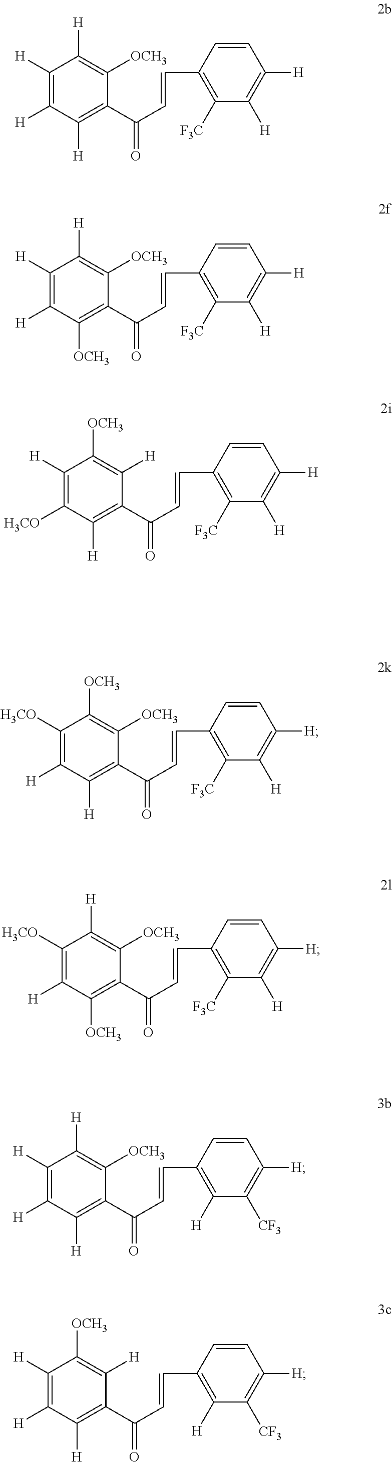

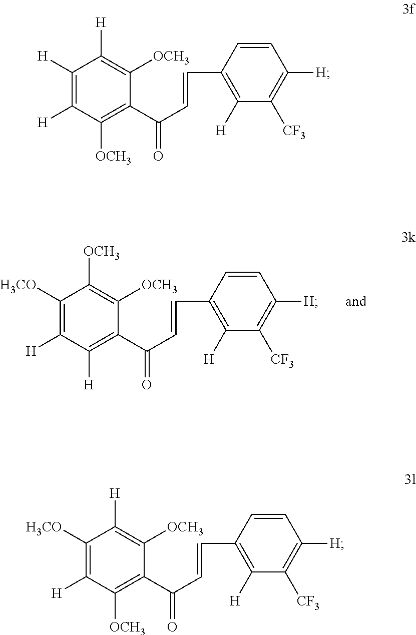

and pharmaceutically acceptable salts thereof.

In yet other embodiments, the compound of Formula (Ia) is selected from the group consisting of:

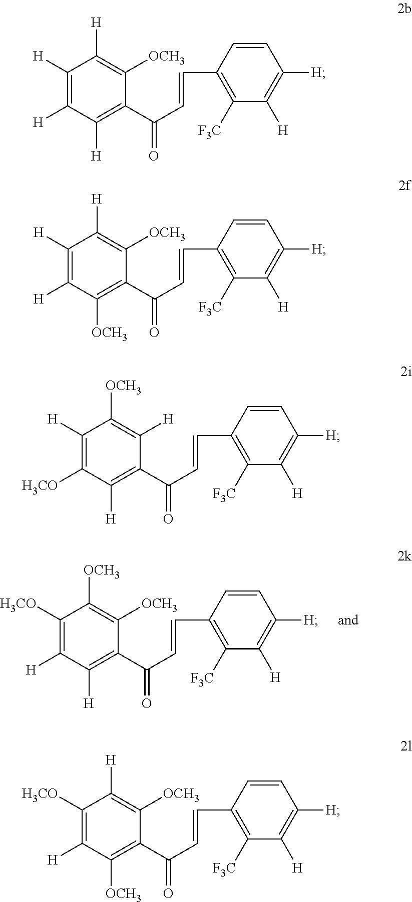

and pharmaceutically acceptable salts thereof.

In particular embodiments, the compound of Formula (Ia) is:

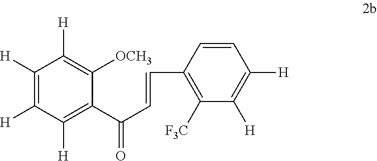 [SMILES: COc1ccccc1C(=O)/C=C/c1ccccc1C(F)(F)F]

and pharmaceutically acceptable salts thereof.

Throughout the specification and claims, a given chemical formula or name shall encompass all tautomers, congeners, and optical- and stereoisomers, as well as racemic mixtures where such isomers and mixtures exist.

B. Pharmaceutical Compositions of Formula (Ia) and Formula (Ib)

In some embodiments, the presently disclosed subject matter provides pharmaceutical composition comprising a compound of Formulae (Ia) or (Ib) and a pharmaceutically acceptable carrier, for example, pharmaceutical composition including one or more compounds of Formula (Ia) or (Ib), alone or in combination with one or more additional therapeutic agents in admixture with a pharmaceutically acceptable excipient. The term “pharmaceutically-acceptable excipient” as used herein means one or more compatible solid or liquid filler, diluents or encapsulating substances that are suitable for administration into a subject. One of skill in the art will recognize that the pharmaceutical compositions include the pharmaceutically acceptable salts of the compounds.

The term “pharmaceutically acceptable salts” is meant to include salts of active compounds which are prepared with relatively nontoxic acids or bases, depending on the particular substituent moieties found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids, such as arginate and the like, and salts of organic acids like glucuronic or galactunoric acids and the like {see, for example, Berge et al, “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

One of ordinary skill in the art would appreciate that certain substituent groups can be added to the presently disclosed compounds to make them amenable to salt formation. For example, acidic functional groups can form stable salts with cations and basic functional groups can form stable salts with acids. Generally, there should be a difference of at least three units in the pKₐ (the logarithmic parameter of the dissociation constant Kₐ, which reflects the degree of ionization of a substance at a particular pH) of the parent drug and the counterion. For parent drug molecules that are very weakly basic, the choice of salt former is preferably a strong acid, such as hydrochloric (pKₐ=−6.1), sulfuric (pKₐ₁=−3.0, pKₐ₂=−1.96), or methanesulfonic (pKₐ=−1.2) to ensure protonation of the parent drug molecule. Parent drug molecules that are more highly basic can form salts with weaker acids, such as phosphoric (pKₐ₁=2.15, pKₐ₂=7.2, pKₐ₃=12.38), tartaric (pKₐ=2.93), acetic (pKₐ=4.76), and benzoic (pKₐ=4.2) acids. For very weakly acidic parent drug molecules, strongly basic cations, such as sodium (pKₐ=14.8), potassium (pKₐ=16.0), or calcium (pKₐ=12.9) are preferable to ensure deprotonation of the parent drug molecule. Parent drug molecules that are more acidic can form stable salts with weaker cations, such as zinc (pKₐ=8.96), choline (pKₐ=8.9), and dithanolamine (pKₐ=9.65). Representative functional groups suitable for stable salt formation, listed in view of relative acid/base strength from stronger acid to stronger base, include, but are not limited to, sulphonic acid (pKₐ₁=−1.2, pKₐ₂=−0.7), carboxylic acid (pKₐ₁=4.2, pKₐ₂=−4.7), imide (pKₐ=8.2), phenol, thiol (pKₐ=10), sulphonamide (pKₐ=10-11), amide (pKₐ=13-14), pyridine/pyridyl (pKₐ=5.2), imine (pKₐ=9.2), arylamine (pKₐ=9.3), alkylamine (pKₐ=9.8-11), amidine (pKₐ=12.4), guanidine (pKₐ=13.7), and quaternary ammonium. See Wermuth, C. G., The Practice of Medicinal Chemistry, 3rd ed., Elsevier, pp. 751-755 (2008).

In addition to salt forms, the present disclosure provides compounds, which are in a prodrug form. Prodrugs of the compounds described herein are those compounds that readily undergo chemical changes under physiological conditions to provide the compounds of the present disclosure. Additionally, prodrugs can be converted to the compounds of the present disclosure by chemical or biochemical methods in an ex vivo environment. For example, prodrugs can be slowly converted to the compounds of the present disclosure when placed in a transdermal patch reservoir with a suitable enzyme or chemical reagent.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

The compounds according to the disclosure are effective over a wide dosage range. For example, in treating adult humans, dosages from 0.01 to 1000 mg, from 0.5 to 100 mg, from 1 to 50 mg per day, and from 5 to 40 mg per day are examples of dosages that may be used. The exact dosage will depend upon the route of administration, the form in which the compound is administered, the subject to be treated, the body weight of the subject to be treated, and the preference and experience of the attending physician. Pharmaceutical compositions suitable for use in the present disclosure include compositions wherein the active ingredients are contained in an effective amount to achieve its intended purpose. Determination of the effective amounts is well within the capability of those skilled in the art, especially in light of the detailed disclosure provided herein.

Depending on the specific conditions being treated, such agents may be formulated into liquid or solid dosage forms and administered systemically or locally. The agents may be delivered, for example, in a timed- or sustained-low release form as is known to those skilled in the art. Techniques for formulation and administration may be found in Remington: The Science and Practice of Pharmacy (20^{th} ed.) Lippincott, Williams & Wilkins (2000). Suitable routes may include oral, buccal, by inhalation spray, sublingual, rectal, transdermal, vaginal, transmucosal, nasal or intestinal administration; parenteral delivery, including intramuscular, subcutaneous, intramedullary injections, as well as intrathecal, direct intraventricular, intravenous, intra-articullar, intra-sternal, intra-synovial, intra-hepatic, intralesional, intracranial, intraperitoneal, intranasal, or intraocular injections or other modes of delivery.

For injection, the agents of the disclosure may be formulated and diluted in aqueous solutions, such as in physiologically compatible buffers, such as Hank's solution, Ringer's solution, or physiological saline buffer. For such transmucosal administration, penetrants appropriate to the barrier to be permeated are used in the formulation. Such penetrants are generally known in the art.

Use of pharmaceutically acceptable inert carriers to formulate the compounds herein disclosed for the practice of the disclosure into dosages suitable for systemic administration is within the scope of the disclosure. With proper choice of carrier and suitable manufacturing practice, the compositions of the present disclosure, in particular, those formulated as solutions, may be administered parenterally, such as by intravenous injection. The compounds can be formulated readily using pharmaceutically acceptable carriers well known in the art into dosages suitable for oral administration. Such carriers enable the compounds of the disclosure to be formulated as tablets, pills, capsules, liquids, gels, syrups, slurries, suspensions and the like, for oral ingestion by a subject (e.g., patient) to be treated.

For nasal or inhalation delivery, the agents of the disclosure also may be formulated by methods known to those of skill in the art, and may include, for example, but not limited to, examples of solubilizing, diluting, or dispersing substances, such as, saline, preservatives, such as benzyl alcohol, absorption promoters, and fluorocarbons.

In addition to the active ingredients, these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions.

Pharmaceutical preparations for oral use can be obtained by combining the active compounds with solid excipients, optionally grinding a resulting mixture, and processing the mixture of granules, after adding suitable auxiliaries, if desired, to obtain tablets or dragee cores. Suitable excipients are, in particular, fillers, such as sugars, including lactose, sucrose, mannitol, or sorbitol; cellulose preparations, for example, maize starch, wheat starch, rice starch, potato starch, gelatin, gum tragacanth, methyl cellulose, hydroxypropylmethyl-cellulose, sodium carboxymethyl-cellulose (CMC), and/or polyvinylpyrrolidone (PVP: povidone). If desired, disintegrating agents may be added, such as the cross-linked polyvinylpyrrolidone, agar, or alginic acid or a salt thereof, such as sodium alginate.

Dragee cores are provided with suitable coatings. For this purpose, concentrated sugar solutions may be used, which may optionally contain gum arabic, talc, polyvinylpyrrolidone, carbopol gel, polyethylene glycol (PEG), and/or titanium dioxide, lacquer solutions, and suitable organic solvents or solvent mixtures. Dye-stuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin, and a plasticizer, such as glycerol or sorbitol. The push-fit capsules can contain the active ingredients in admixture with filler, such as lactose, binders, such as starches, and/or lubricants, such as talc or magnesium stearate and, optionally, stabilizers. In soft capsules, the active compounds may be dissolved or suspended in suitable liquids, such as fatty oils, liquid paraffin, or liquid polyethylene glycols (PEGs). In addition, stabilizers may be added.

In particular embodiments, the pharmaceutical composition further comprises one or more agents selected from the group consisting of a corticosteroid, an antibiotic, and combinations thereof. In particular embodiments, the corticosteroid is selected from the group consisting of dexamethasone, flunisolide, fluticasone propionate, triamcinolone acetonide, beclomethasone dipropionate, budesonide, prednisone, prednisolone, and methylprednisolone. In some embodiments, the pharmaceutical composition is formulated for inhalation or oral administration.

In some embodiments, the presently disclosed subject matter provides a kit for treating or preventing a radiation injury, the kit comprising a therapeutically effect amount of compound of Formula (Ia) and written instructions for use of the kit.

In yet further embodiments, the presently disclosed subject matter provides a device for dispersing one or more particles comprising a compound of Formula (Ia) in an amount effective to increase a Nrf2 biological activity or Nrf2 expression and delivering a dose of the particles to lung tissue of a subject. In particular embodiments, the device is selected from the group consisting of a nebulizer, a metered dose inhaler, and a dry powder inhaler.

B. Methods for Treating or Preventing a Disease, Disorder, or Condition Associated with an Nrf2-Regulated Pathway

In some embodiments, and as disclosed in more detail herein below, the presently disclosed subject matter provides a method for treating or preventing a disease, disorder or condition associated with an Nrf2-regulated pathway, the method comprising administering a compound of Formula (Ib) to the subject in an amount effective to increase an Nrf2 biological activity or Nrf2 expression, thereby treating or preventing the disease, disorder, or condition:

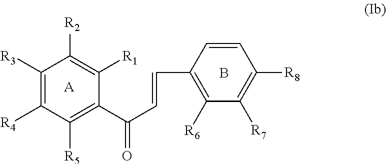

wherein: R₁, R₂, R₃, R₄, and R₅ are each independently selected from the group consisting of H and alkoxyl, provided that at least one of R₁, R₂, R₃, R₄, and R₅ is alkoxyl; R₆, R₇, and R₈ are each independently selected from the group consisting of H, CF₃, and NO₂, provided that at least one of R₆, R₇, and R₈ is CF₃ or NO₂; and pharmaceutically acceptable salts thereof.

By “Nrf2 expression or biological activity” is meant binding to an antioxidant-response element (ARE), nuclear accumulation, the transcriptional induction of target genes, or binding to a Keap1 polypeptide.

In some embodiments, the compound of Formula (Ib) is selected from the group consisting of:

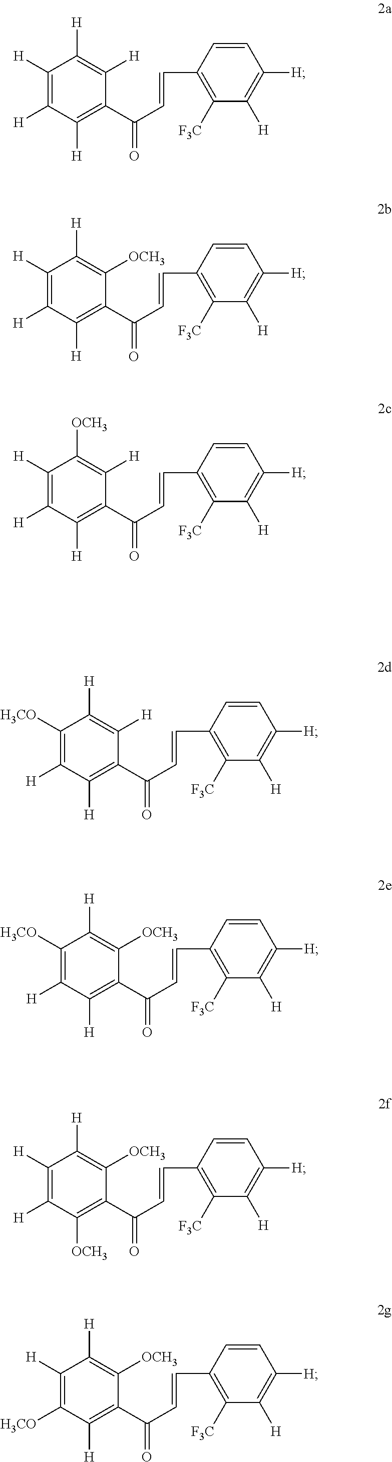

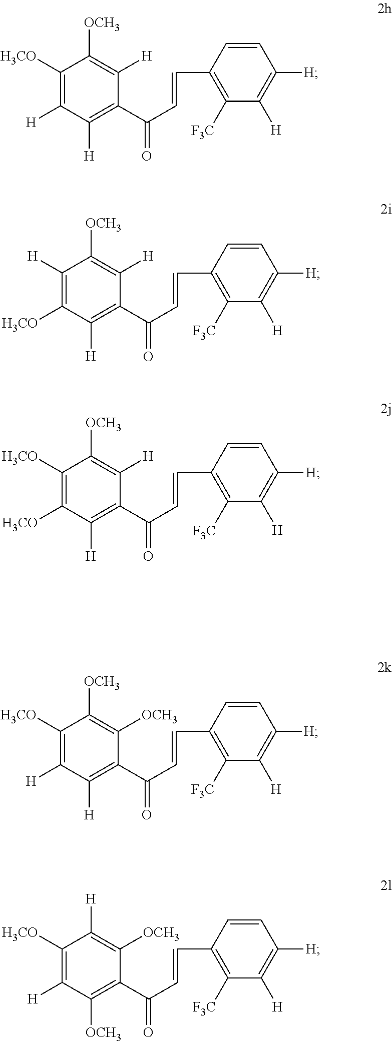

and pharmaceutically acceptable salts thereof.

In yet other embodiments, the compound of Formula (Ib) is selected from the group consisting of:

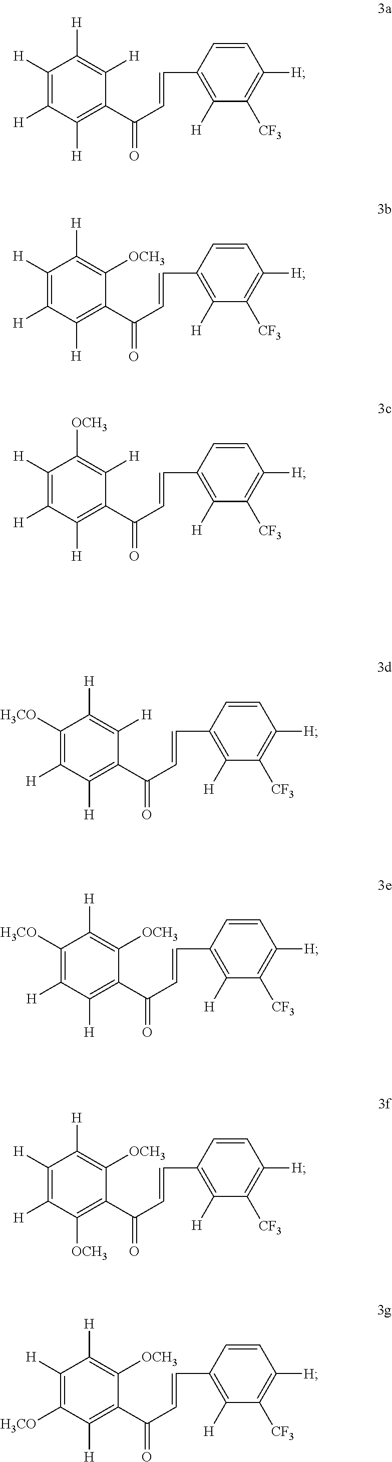

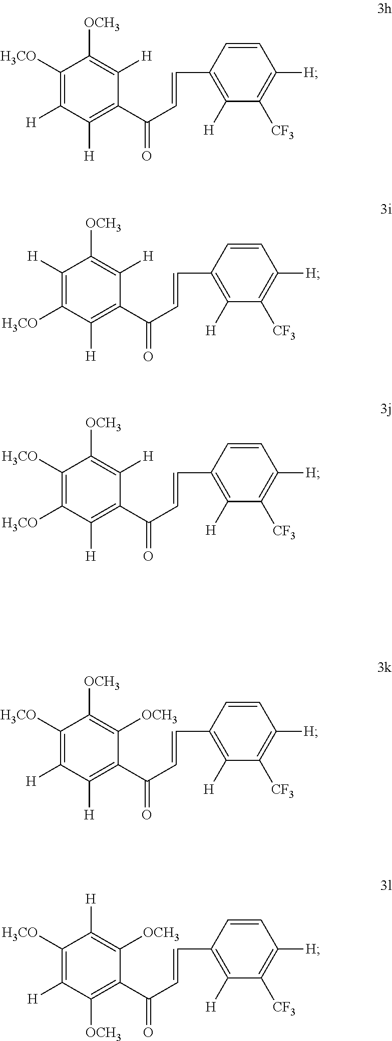

and pharmaceutically acceptable salts thereof.

In further embodiments, the compound of Formula (Ib) is selected from the group consisting of:

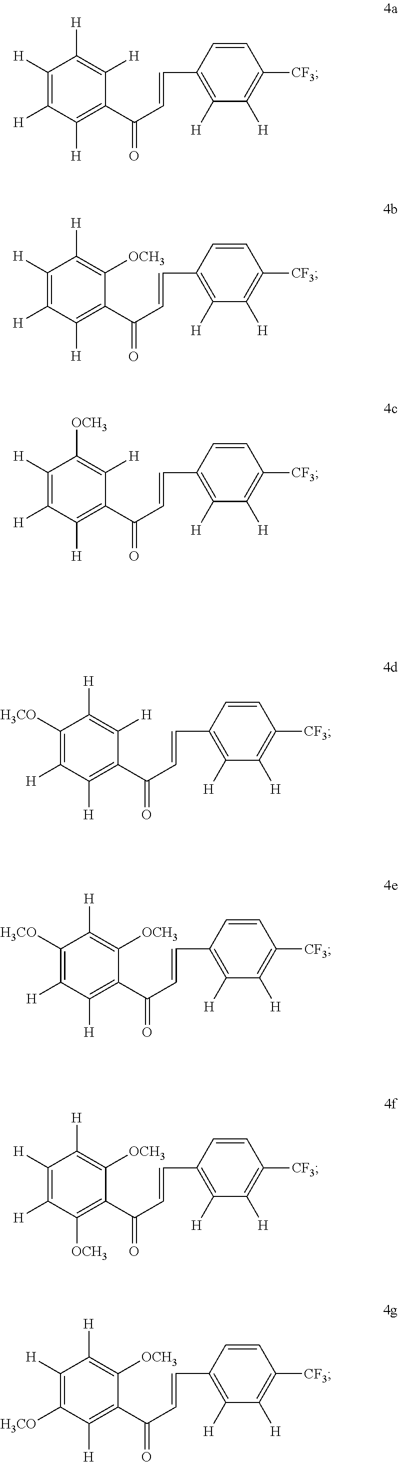

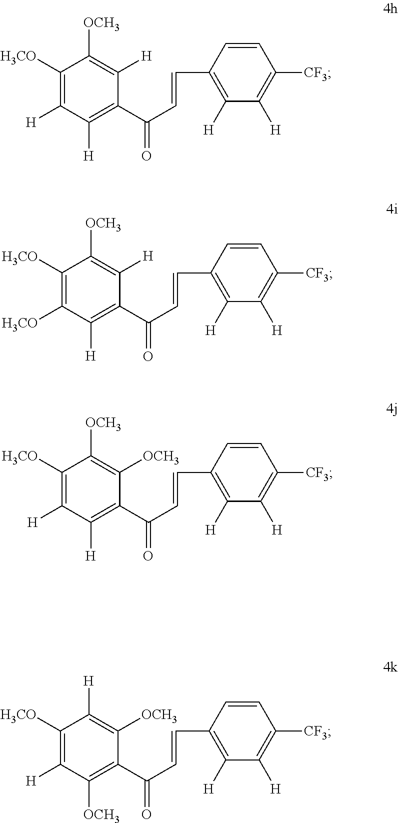 [SMILES: COc1ccc(C(=O)/C=C/c2ccc(C(F)(F)F)cc2)cc1OC ; COc1cc(C(=O)/C=C/c2ccc(C(F)(F)F)cc2)cc(OC)c1OC ; COc1ccc(C(=O)/C=C/c2ccc(C(F)(F)F)cc2)c(OC)c1OC ; COc1cc(OC)c(C(=O)/C=C/c2ccc(C(F)(F)F)cc2)c(OC)c1]

and pharmaceutically acceptable salts thereof.

In particular embodiments, the compound of Formula (Ib) is:

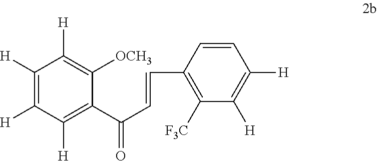 [SMILES: COc1ccccc1C(=O)/C=C/c1ccccc1C(F)(F)F]

and pharmaceutically acceptable salts thereof.

As used herein, the terms “treat,” treating,” “treatment,” and the like, are meant to decrease, suppress, attenuate, diminish, arrest, the underlying cause of a disease, disorder, or condition, or to stabilize the development or progression of a disease, disorder, condition, and/or symptoms associated therewith. It will be appreciated that, although not precluded, treating a disease, disorder or condition does not require that the disease, disorder, condition or symptoms associated therewith be completely eliminated.

As used herein, the terms “prevent,” “preventing,” “prevention,” “prophylactic treatment” and the like refer to reducing the probability of developing a disease, disorder, or condition in a subject, who does not have, but is at risk of or susceptible to developing a disease, disorder, or condition. Thus, in some embodiments, an agent can be administered prophylactically to prevent the onset of a disease, disorder, or condition, or to prevent the recurrence of a disease, disorder, or condition.

By “agent” is meant a compound of Formula (Ib) or another agent, e.g., a peptide, nucleic acid molecule, or other small molecule compound administered in combination with a compound of Formula (Ib).

More particularly, the term “therapeutic agent” means a substance that has the potential of affecting the function of an organism. Such an agent may be, for example, a naturally occurring, semi-synthetic, or synthetic agent. For example, the therapeutic agent may be a drug that targets a specific function of an organism. A therapeutic agent also may be an antibiotic or a nutrient. A therapeutic agent may decrease, suppress, attenuate, diminish, arrest, or stabilize the development or progression of disease, disorder, or condition in a host organism.

The term “effective amount” of a therapeutic agent refers to the amount of the agent necessary to elicit the desired biological response. As will be appreciated by those of ordinary skill in this art, the effective amount of an agent may vary depending on such factors as the desired biological endpoint, the agent to be delivered, the composition of the pharmaceutical composition, the target tissue or cell, and the like. More particularly, the term “effective amount” refers to an amount sufficient to produce the desired effect, e.g., to reduce or ameliorate the severity, duration, progression, or onset of a disease, disorder, or condition, or one or more symptoms thereof; prevent the advancement of a disease, disorder, or condition, cause the regression of a disease, disorder, or condition; prevent the recurrence, development, onset or progression of a symptom associated with a disease, disorder, or condition, or enhance or improve the prophylactic or therapeutic effect(s) of another therapy.

An effective amount of a compound according to the presently disclosed methods can range from, e.g., about 0.001 mg/kg to about 1000 mg/kg, or in certain embodiments, about 0.01 mg/kg to about 100 mg/kg, or in certain embodiments, about 0.1 mg/kg to about 50 mg/kg. Effective doses also will vary, as recognized by those skilled in the art, depending on the disorder treated, route of administration, excipient usage, the age and sex of the subject, and the possibility of co-usage with other therapeutic treatments, such as use of other agents. It will be appreciated that an amount of a compound required for achieving the desired biological response may be different from the amount of compound effective for another purpose.

By “in combination with” is meant the administration of a compound of Formula (Ib) with one or more therapeutic agents either simultaneously, sequentially, or a combination thereof. Therefore, a cell or a subject administered a combination of a compound of Formula (Ib) can receive a compound of Formula (Ib) and one or more therapeutic agents at the same time (i.e., simultaneously) or at different times (i.e., sequentially, in either order, on the same day or on different days), so long as the effect of the combination of both agents is achieved in the cell or the subject. When administered sequentially, the agents can be administered within 1, 5, 10, 30, 60, 120, 180, 240 minutes or longer of one another. In other embodiments, agents administered sequentially, can be administered within 1, 5, 10, 15, 20 or more days of one another. Where the compound of Formula (Ib) and one or more therapeutic agents are administered simultaneously, they can be administered to the cell or administered to the subject as separate pharmaceutical compositions, each comprising either a compound of Formula (Ib) or one or more therapeutic agents, or they can contact the cell as a single composition or be administered to a subject as a single pharmaceutical composition comprising both agents.

When administered in combination, the effective concentration of each of the agents to elicit a particular biological response may be less than the effective concentration of each agent when administered alone, thereby allowing a reduction in the dose of one or more of the agents relative to the dose that would be needed if the agent was administered as a single agent. The effects of multiple agents may, but need not be, additive or synergistic. The agents may be administered multiple times.

The subject treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for treating an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein.

1. Methods of Treating an Autoimmune Disease

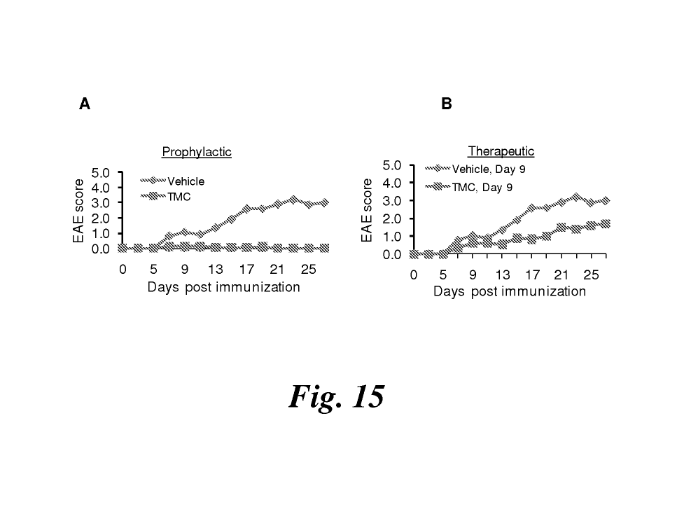

In some embodiments, the presently disclosed subject matter provides a method for treating or preventing an autoimmune disease, disorder or condition associated with an Nrf2-regulated pathway, the method comprising administering a compound of Formula (Ib) to the subject in an amount effective to increase an Nrf2 biological activity or Nrf2 expression, thereby treating or preventing the autoimmune disease, disorder, or condition. Without wishing to be bound to any one particular theory, it is believed that the presently disclosed compounds act as potent immunomodulators, which upon activation of the Nrf2 signaling pathway, protect a subject from an autoimmune disease by suppressing inflammation. For example, as provided in more detail herein below, the data in FIG. 15 related to multiple sclerosis indicate that the presently disclosed activators of Nrf2 also are immunomodulators.

In some embodiments, the autoimmune disease is selected from the group consisting of acute graft-versus host disease, autoimmune inner ear disease, inflammatory bowel disease, rheumatoid arthritis, psoriasis, psoriatic arthritis, multiple sclerosis, scleroderma, lupus, ankylosing spondylitis, neutropenia, and uveitis. Again, without wishing to be bound to any one particular theory, it is believed that these diseases are mainly mediated by Th1 and Th17 inflammation and activating the Nrf2 pathway can suppress these inflammatory mediators.

In other embodiments, the presently disclosed subject matter provides compositions and methods for treating or preventing a comorbidity associated with diabetes including, but not limited to, retinopathy and nephropathy. As used herein, the term “comorbidity” includes either the presence of one or more disorders (or diseases) in addition to a primary disease or disorder, or the effect of such additional disorders or diseases on a subject. “Comorbidity” can include (i) a medical condition existing simultaneously, but independently with another condition in a subject; and/or (ii) a medical condition in a subject that causes, is caused by, or is otherwise related to another condition in the same subject.

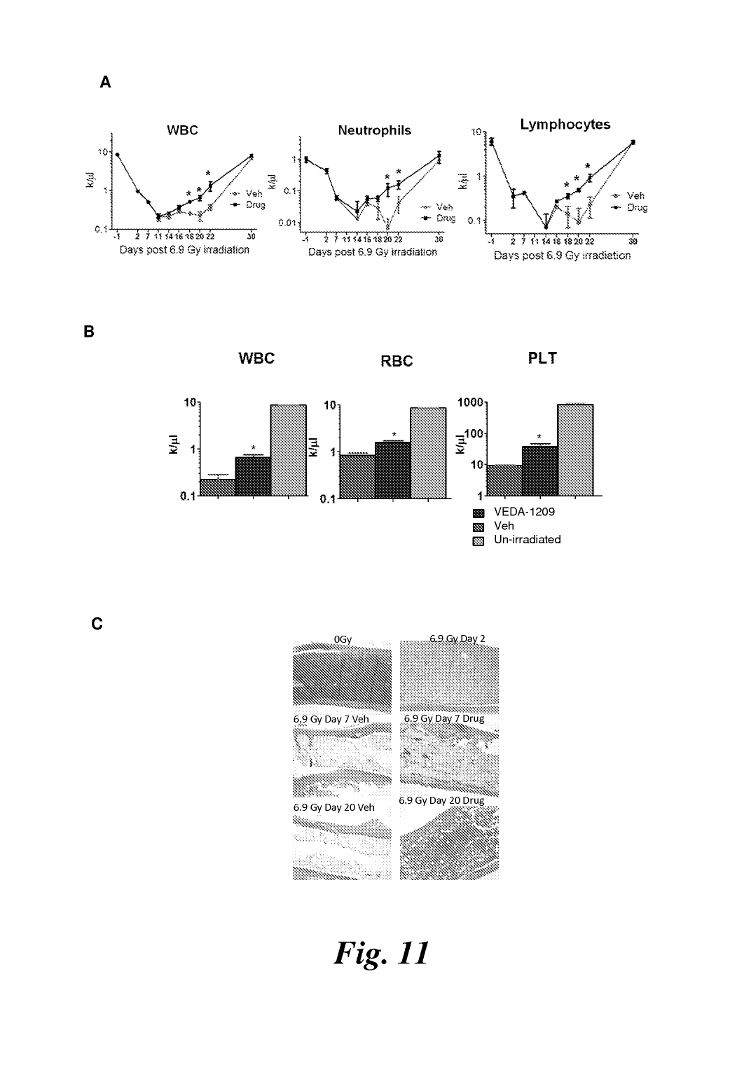

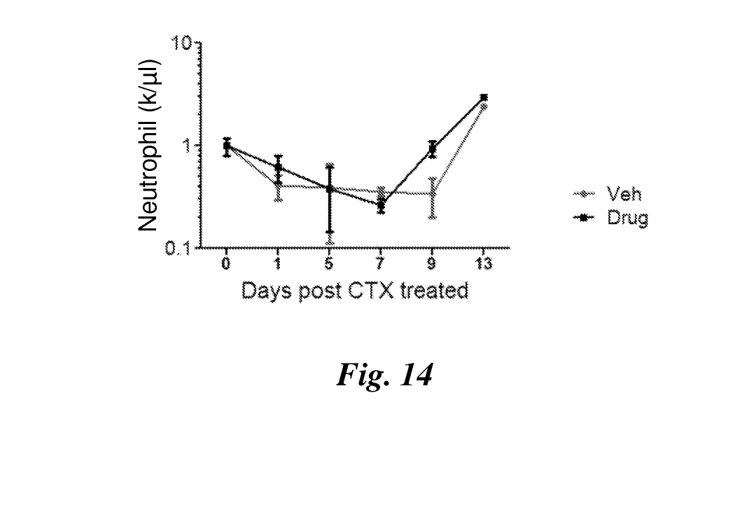

In yet other embodiments, the presently disclosed subject matter provides compositions and methods for improving the outcome for bone marrow transplant for leukemia and related cancers and treating bone marrow deficiencies, inborn errors of metabolism, and immune disorders. Activating Nrf2 also is thought to stimulate hematopoiesis, see, e.g., FIG. 11 and FIG. 14, as well as, Merchant, A A, et al., The redox-sensitive transcription factor Nrf2 regulates murine hematopoietic stem cell survival independently of ROS levels, Blood 2011; 118(25):6572-6579.

2. Methods of Treating a Disease or Condition Associated with Oxidative Stress

Methods of treating a disease, disorder, or condition associated with oxidative stress are disclosed in International PCT Patent Application Publication No. WO2007/005879, which is incorporated herein by reference in its entirety. Oxidative Stress describes the level of oxidative damage caused by reactive oxygen species (ROS) in a cell, tissue, or organ. Reactive oxygen species (e.g., free radicals, reactive anions) are generated in endogenous metabolic reactions. Exogenous sources of reactive oxygen species include exposure to cigarette smoke and environmental pollutants. Reactions between free radicals and cellular components result in the alteration of macromolecules, such as polyunsaturated fatty acids in membrane lipids, essential proteins, and DNA. Oxidative stress results when the formation of free radicals exceeds antioxidant activity. Accordingly, by “oxidative stress” is meant cellular damage or a molecular alteration in response to a reactive oxygen species. By “disease or disorder related to oxidative stress” is meant any pathology characterized by an increase in oxidative stress. Oxidative stress is implicated in a variety of disease states, including Alzheimer's disease, Parkinson's disease, inflammatory diseases, neurodegenerative diseases, heart disease, HIV disease, chronic fatigue syndrome, hepatitis, cancer, autoimmune diseases, and aging.

In some embodiments, the presently disclosed subject matter provides a method for treating a disease, disorder, or condition associated with oxidative stress. Mammals having reduced levels of Nrf2 are particularly susceptible to tissue damage associated with oxidative stress, including pulmonary inflammatory conditions, sepsis, and neuronal cell death associated with ischemic injury. Nrf2 provides protection against oxidative stress and reduces neuronal cell death associated with ischemic injury. Accordingly, agents that increase the expression or biological activity of Nfr2 are useful for preventing and treating diseases or disorders associated with increased levels of oxidative stress or reduced levels of antioxidants, including pulmonary inflammatory conditions, pulmonary fibrosis, asthma, chronic obstructive pulmonary disease (COPD), acute respiratory distress syndrome (ARDS), emphysema, sepsis, septic shock, ischemic injury, including cerebral ischemia and heart ischemia, cognitive deficits, and neurodegenerative disorders.

3. Oxidative Stress and Pulmonary Disorders

Oxidative stress is involved in the pathogenesis of pulmonary diseases, including asthma, COPD, and emphysema. By “pulmonary inflammatory condition” is meant any disease, disorder, or condition characterized by an increase in airway inflammation, intermittent reversible airway obstruction, airway hyperreactivity, excessive mucus production, or an increase in cytokine production (e.g., elevated levels of immunoglobulin E and Th2 cytokines).

In particular, increased Nrf2 activation is associated with a decrease in airway remodeling (Rangasamy, T., et al., Disruption of Nrf2 enhances susceptibility to severe airway inflammation and asthma in mice. J. Exp. Med. 2005, 202, 47-59). Airway remodeling occurs as a result of the proliferation of fibroblasts. Increased remodeling is associated with several pulmonary diseases, such as COPD, asthma and interstitial pulmonary fibrosis (IPF). Compounds and strategies that increase Nrf2 biological activity or expression are useful for preventing or decreasing fibrosis and airway remodeling in lungs as a result of COPD, asthma and IPF.

The lungs of Nrf2^{-/-} mice exhibit a defective antioxidant response that leads to worsened asthma, exacerbates airway inflammation and increases airway hyperreactivity (AHR). By “antioxidant response” is meant an increase in the expression or activity of a Nrf2 regulated gene. Exemplary Nrf2 regulated genes are described herein (see Table 1). Critical host factors that protect the lungs against oxidative stress determine susceptibility to asthma or act as modifiers of risk by inhibiting associated inflammation. Nrf2-regulated genes in the lungs include almost all of the relevant antioxidants, such as heme oxygenase-1 (HO-I), γ-glutamyl cysteine synthase (γ-GCS), and several members of the GST family. Methods for increasing Nrf-2 expression or biological activity are, therefore, useful for treating pulmonary diseases associated with oxidative stress, inflammation, and fibrosis. Such diseases include, but are not limited to, chronic bronchitis, emphysema, inflammation of the lungs, pulmonary fibrosis, interstitial lung diseases, and other pulmonary diseases or disorders characterized by subepithelial fibrosis, mucus metaplasia, and other structural alterations associated with airway remodeling.

4. Ischemia and Neurodegenerative Disease

Nrf2 protects cells and multiple tissues by coordinately up-regulating ARE-related detoxification and antioxidant genes and molecules required for the defense system in each specific environment. A role has been identified for Nrf2 as a neuroprotectant molecule that reduces apoptosis in neural tissues following transient ischemia. By “ischemic injury” is meant any negative alteration in the function of a cell, tissue, or organ in response to hypoxia. By “reperfusion injury” is meant any negative alteration in the function of a cell, tissue, or organ in response restore of blood flow following transient occlusion.

Accordingly, in some embodiments, the presently disclosed subject matter provides compositions and methods for treating a variety of disorders involving cell death, including but not limited to, neuronal cell death. In one embodiment, agents that increase Nrf2 expression or biological activity are useful for treating or preventing a disease or disorder characterized by increased levels of cell death, including ischemic injury (caused by, e.g., a myocardial infarction, a stroke, or a reperfusion injury, brain injury, stroke, and multiple infarct dementia, a secondary exsanguination or blood flow interruption resulting from any other primary diseases), as well as neurodegenerative disorders (e.g., Alzheimer's disease (AD) Creutzfeldt-Jakob disease, Huntington's disease, Lewy body disease, Pick's disease, Parkinson's disease, amyotrophic lateral sclerosis (ALS), multiple sclerosis (MS), and neurofibromatosis). By “neurodegenerative disorder” is meant any disease or disorder characterized by increased neuronal cell death, including neuronal apoptosis or neuronal necrosis.

5. Methods for Reversing Corticosteroid Resistance and Treating Respiratory Infections

Methods for reversing corticosteroid resistance and treating respiratory infections are disclosed in International PCT Patent Application Publication No. WO2011/094598, which is incorporated herein by reference in its entirety. By “corticosteroid resistance” is meant having diminished corticosteroid sensitivity.

Chronic obstructive pulmonary disease (COPD) is characterized by a progressive decrease in lung function and encompasses both chronic bronchitis and emphysema. COPD is the fifth leading cause of death worldwide. Tobacco exposure is the major risk factor for COPD development in industrialized countries. Patients with COPD have frequent symptomatic exacerbations, which are primarily due to exposure to bacterial or viral infections or environmental pollutants. These exacerbations are a major cause of morbidity, mortality, and healthcare costs.

Among the bacterial causes of exacerbations, nontypeable Haemophilus influenzae (NTHI) is the most prevalent, and Pseudomonas aeruginosa (PA) becomes important in severe COPD. Patients with advanced COPD experience, on average, two to three periods of exacerbation annually. Clinical and animal studies have shown that cigarette smoking causes defective bacterial phagocytosis by alveolar macrophages resulting in bacterial colonization and enhanced inflammation in lungs. Currently, there are no proven therapies that can inhibit bacterial colonization and prevent infectious COPD exacerbations.

Current treatments for COPD are of limited benefit. Corticosteroids are highly effective anti-inflammatory drugs for asthma, but they have little therapeutic benefit in COPD because of diminished corticosteroid sensitivity. High doses of inhaled corticosteroids are widely used to manage COPD; but they reduce exacerbations by only about 20% to 25% and do not alter disease progression or survival. High doses of systemic corticosteroids are used to treat acute severe COPD exacerbations, but they reduce length of hospitalization by only 9%.

Corticosteroid resistance due to inactivation of histone deacetylase (HDAC) 2 is a barrier to effective treatment of chronic obstructive pulmonary disease (COPD). The presently disclosed subject matter is based, at least in part, on the discovery that S-nitrosylation is a key posttranslational modification responsible for inactivation of HDAC2 in COPD alveolar macrophages that can be reversed by targeting transcription factor nuclear factor erythroid 2-related factor 2 (Nrf2). Sulforaphane, a small-molecule activator of Nrf2, restores the function of HDAC2 by denitrosylation in a glutathione-dependent manner, thereby augmenting deacetylation of histones in the interleukin-8 promoter and glucocorticoid receptor in alveolar macrophages from patients with COPD. In contrast to nitric oxide synthase inhibition alone, sulforaphane treatment reestablishes the repressive effect of corticosteroid on cytokine production in alveolar macrophages from patients with COPD. Sulforaphane restores HDAC2 function and corticosteroid sensitivity in alveolar macrophages from cigarette smoke-exposed mice. Thus, Nrf2 is a novel drug target to reverse corticosteroid resistance in COPD and other corticosteroid-resistant inflammatory diseases (e.g., severe asthma, acute graft-versus host disease, autoimmune inner ear disease, inflammatory bowel diseases, and rheumatoid arthritis).

Patients with chronic obstructive pulmonary disease (COPD) have pulmonary innate immune dysfunction largely due to defective macrophage phagocytic ability by unknown mechanisms. This condition results in periodic bacterial infection and colonization that cause acute exacerbation of COPD, a major source for morbidity and mortality. The presently disclosed subject matter is further based, at least in part, on the discovery that activation of transcription factor Nrf2 by sulforaphane treatment restores bacterial recognition, phagocytic ability and clearance of clinical isolates nontypeable Haemophilus influenza (NTHI) and Pseudomonas aeruginosa (PA) by alveolar macrophages from patients with COPD. Molecular studies reveal Nrf2 improves macrophage phagocytic ability by direct transcriptional upregulation of class A scavenger receptor MARCO and was independent of its antioxidant function. Sulforaphane treatment restored phagocytic ability of alveolar macrophages by increasing MARCO and inhibited bacterial colonization (NTHI or PA) and inflammation in the lungs of wild-type mice after 6 months of chronic exposure to cigarette smoke. These findings identify increasing MARCO by targeting Nrf2 as a therapeutic approach to improve anti-bacterial defenses and suggest that this pathway can be targeted for preventing bacterial exacerbations in COPD.

Accordingly, agents that increase the expression or biological activity of Nfr2 (e.g., compounds of Formula (Ib)) are useful for reversing corticosteroid resistance, as well as for treating respiratory infections, particularly those associated with chronic obstructive pulmonary disease, emphysema, and related conditions. Therefore, the presently disclosed subject matter provides compositions for reversing corticosteroid resistance that comprise an agent that increases Nrf2 activity, alone or in combination with a corticosteroid (e.g., dexamethasone, flunisolide, fluticasone propionate, triamcinolone acetonide, beclomethasone dipropionate, budesonide, prednisone, prednisolone, and methylprednisolone).

In other embodiments, the presently disclosed subject matter provides compositions for treating a bacterial infection, particularly for bacterial infections that occur in a subject having or at risk of developing COPD, in subjects having chronic bronchitis, in smokers, and in subjects having cystic fibrosis or having an immunodeficiency syndrome that reduces or otherwise compromises the efficacy of the subject's immune system.

Conditions associated with corticosteroid resistance include, but are not limited to, corticosteroid resistance in COPD, asthma, including severe asthma, acute graft-versus host disease, autoimmune inner ear disease, inflammatory bowel diseases, rheumatoid arthritis, as well as bacterial infections, including those associated with COPD and related conditions (e.g., smoking, chronic bronchitis).

By “pulmonary inflammatory condition” also is meant any pathological condition that increases mononuclear cells (monocytes/macrophages, lymphocytes), neutrophils, and fibroblasts in the lungs. Exemplary pulmonary inflammatory conditions include, but are not limited to, bacterial, viral, or fungal pulmonary infections, environmental pollutants (e.g., particulate matter, automobile exhaust, allergens), chronic obstructive pulmonary disease, asthma, acute lung injury/acute respiratory distress syndrome or inflammation.

By “restoring corticosteroid responsiveness” is meant increasing the anti-inflammatory action of corticosteroids in subjects having reduced sensitivity to corticosteroid treatment. The restoration need not be complete, but can be an increase in sensitivity of at least about 10%, 25%, 30%, 50%, 75% or more.

By “reversing corticosteroid insensitivity” is meant re-establishing the repressive effect of corticosteroids on cytokine production in subjects having reduced sensitivity to corticosteroid treatment, thereby reducing the levels required for efficacy to those closer to levels typically used in subjects that are not corticosteroid insensitive.

By “respiratory infection” is meant any infection affecting the respiratory system (e.g., lungs and associated tissues). Exemplary respiratory infections include, but are not limited to, infections with a Gram negative or positive bacteria (e.g., Pseudomonas aeruginosa, nontypeable Haemophilus influenzae, Moraxella catarrhalis, Streptococcus pneumonia, Staphylococcus aureus), or a virus (e.g., Rhinovirus, coronovirus, influenza A and B, parainfluenza, Adenovirus, and Respiratory syncytial virus).

6. Methods for Treating Radiation Damage

Radiation injury can occur from external irradiation, either when the entire body is irradiated or when only part of the body is irradiated. Radiation injury may occur in connection with radiotherapy, during an accidental exposure to radioactivity, e.g., nuclear fallout from a nuclear accident, or in connection with a nuclear attack. Accidental exposure or nuclear attack also can cause internal radiation exposure due to widespread radioactive particles released in the environment. Radiation exposure causes short term and/or long term disorders. Clinical components of the acute radiation syndrome include hematopoietic, gastrointestinal, and cerebrovascular syndromes that occur within days to a few weeks following radiation exposure. Long term disorders, such as lung fibrosis, following radiation exposure are typically associated with tissue damage.

Health effects after radiation exposure are caused by damage to rapidly dividing normal cells. Therapies directed toward preventing and mitigating injuries caused by unintentional (e.g., nuclear accidents or attack) or intentional (e.g., cancer treatment) radiation exposure are vital to addressing such health effects.

In the case of whole body exposure to high doses of radiation, acute radiation syndrome can appear within minutes or days after radiation exposure. At doses between 2 Gy and 6 Gy, the hematopoietic system is significantly damaged leading to immunosuppression, infection, and bleeding. Without appropriate therapy, death may result within 60 days. At doses higher than 6 Gy, significant damage to the gut occurs, which results in severe nausea, vomiting, diarrhea, ulceration of the intestinal mucosa, and systemic infection leading to sepsis. At these doses, death may occur within two to four weeks. Doses higher than 20 Gy cause significant damage to the central nervous system and cardiovascular system that may result in death within two days.

In the case of curative intent, such as eliminating a tumor and/or preventing cancer reoccurrence, the radiation dose for a solid epithelial tumor ranges from about 60 Gy about 80 Gy. During palliative treatment, however, such as relieving suffering by shrinking a tumor in the brain or esophagus, cancer patients are exposed to about 20 Gy to about 40 Gy in 2 Gy fractions. Radiotherapy can cause both acute and late side effects. Depending on the area of the body treated, the acute and late health effects after radiotherapy include permanent damage to salivary glands (e.g., dry mouth and loss of taste), damage to mucosal areas (e.g., oral mucositis, GI mucositis and esophagitis), damage to lungs (e.g., pneumonitis and fibrosis) and damage to the brain (e.g., memory loss).

Toxic effects of radiation are initiated by oxidative stress, which causes cell death, tissue damage, and promotes inflammation. Agents that can inhibit oxidative stress (anti-oxidants), cell death (anti-apoptotic agents or cell survival factors), and inflammation (anti-inflammatory agents) are potential drugs to limit radiation injuries. “Radioprotectants” are agents that protect from radiation damage when administered prior to radiation exposure. “Radiomitigators” are agents that reduce the radiation damage when administered after radiation exposure. In an emergency scenario, radiomitigators that are effective after radiation exposure (e.g., 24 h after radiation exposure) are promising drug candidates.

Methods for treating radiation damage are disclosed in International PCT Patent Application No. WO2010/059245, which is incorporated herein by reference in its entirety. By “radiation injury” is meant any cell, tissue, or organ damage associated with exposure to ionizing radiation. Examples of radiation injury include, but are not limited to, hematopoietic syndrome, gastrointestinal syndrome, cerebrovascular syndrome, cerebrospinal injury, pulmonary effects, sepsis, renal failure, pneumonitis, mucositis, enteritis, fibrosis, skin injuries, neutropenia, and an effect on a soft tissue.

Accordingly, in some embodiments, the presently disclosed subject matter provides compositions and methods that are useful for treating or preventing radiation injury. The presently disclosed subject matter is based, at least in part, on the discovery that compounds that activate Nrf2 protect against cell and tissue damage associated with radiation exposure, and reduce mortality in response to such injury.

a. Radiation Injury

Clinical components of acute radiation syndrome include hematopoietic, gastrointestinal, and cerebrovascular syndromes that occur within days or weeks of exposure. The hematopoietic syndrome, which is characterized by hypoplasia or aplasia of the bone marrow, occurs in connection with significant partial-body or whole-body radiation exposures. These hematopoietic changes result in pancytopenia, predisposition to infection, bleeding, and poor wound healing. Any one of these effects of radiation on hematopoiesis can be fatal. Gastrointestinal syndrome is characterized by abdominal pain, diarrhea, and nausea and vomiting and predispose patients to infection. Radiation induces loss of intestinal crypts and breakdown of the mucosal bather. Cutaneous injury from thermal or radiation burns is characterized by loss of epidermis and dermis. Injuries to the skin may cover small areas, but extend deep into the soft tissue, even reaching underlying muscle and bone.

b. Mechanism of Radiation Injury

ROS and electrophiles generated by irradiation are key players in causing acute and chronic pathological injury. ROS induce oxidative damage to biomolecules and causes apoptosis of hematopoietic cells, endothelial cells and epithelial cells. Depletion of hematopoietic cells in a subject results in an impaired immune response and predisposes the subject to secondary infections. The increased death of endothelial cells and epithelial cells results in a loss of mucosal barrier and tissue injury. Loss of intestinal or lung mucosal barrier leads to translocation of bacteria into systemic circulation and causes systemic inflammation and sepsis. Tissue injury causes local inflammation leading to tissue remodeling and fibrosis. In sum, irradiation increases oxidative stress, apoptosis, and inflammation leading to multi-organ injury, which is often lethal. Therapies directed toward blocking ROS-induced deleterious effects mitigates and treats radiation injury.

In other embodiments, the presently discloses subject matter provides compositions and methods that are useful for treating or preventing chemotherapy injury. By “chemotherapy injury”, it is meant injury or side effects resulting from administering chemotherapy drugs to a patient, such as a cancer patient and the like. The presently disclosed subject matter is based, at least in part, on the discovery that compounds that activate Nrf2 protect against cell and tissue damage associated with chemotherapy exposure.

c. Methods of Treating Neutropenia

Accordingly, in some embodiments, the presently disclosed subject matter provides a method for treating of preventing neutropenia caused by chemotherapy, autoimmunity diseases, and in subjects having a congenital neutropenic disorder. Neutropenia is a condition in which the number of neutrophils in the bloodstream is decreased. Neutrophils are a type of white blood cell, also known as polymorphonuclear leukocytes. Neutropenia can affect the body's ability to fight an infection. As provided hereinabove, activating Nrf2 also is thought to stimulate hematopoiesis, see, e.g., FIG. 11 and FIG. 14, as well as, Merchant, A A, et al., The redox-sensitive transcription factor Nrf2 regulates murine hematopoietic stem cell survival independently of ROS levels, Blood 2011; 118(25):6572-6579.

Unless defined otherwise, all technical and scientific terms used herein have the meaning commonly understood by a person skilled in the art to which this invention belongs.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, parameters, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

### Chalcone Derivatives as Nrf2 Activators in Mice and Human Lung Epithelial Cells

Nuclear factor-erythroid 2 p45-related factor 2 (Nrf2) is a basic-leucine zipper (b-ZIP) transcription factor present in the cytoplasm of normal cells. Upon activation in response to inflammatory stimuli, environmental toxicants, or oxidative and electrophilic stress, Nrf2 detaches from its cytosolic inhibitor, Kelch-like ECH-associated protein 1 (Keap1), and translocates to the nucleus and binds to the antioxidant response element (ARE) of target genes along with other binding partners leading to their transcriptional induction. Kensler, T. W., et al., Cell survival responses to environmental stresses via the Keap1-Nrf2-ARE pathway. Annu. Rev. Pharmacool. Toxicol. 2007, 47, 89-116; Rangasamy, T., et al., Disruption of Nrf2 enhances susceptibility to severe airway inflammation and asthma in mice. J. Exp. Med. 2005, 202, 47-59; Sussan, T. E., et al., Targeting Nrf2 with the triterpenoid CDDO-imidazolide attenuates cigarette smoke-induced emphysema and cardiac dysfunction in mice. Proc. Natl. Acad. Sci. U.S.A. 2009, 106, 250-255; Thimmulappa, R. K., et al., Identification of Nrf2-regulated genes induced by the chemopreventive agent sulforaphane by oligonucleotide microarray. Cancer Res. 2002, 62, 5196-5203.

The Keap1-Nrf2 system is the major regulatory pathway of cytoprotective gene expression against oxidative and/or electrophilic stresses. Keap1 acts as a stress sensor protein in this system. While Keap1 constitutively suppresses Nrf2 activity under unstressed conditions, oxidants or electrophiles provoke the repression of Keap1 activity, thereby inducing the Nrf2 activation. Misra, V., et al., Global expression profiles from C57BL/6J and DBA/2J mouse lungs to determine aging-related genes. Physiological Genomics. 2007, 31, 429-440; Surh, Y. J., et al., Nrf2 as a Master Redox Switch in Turning on the Cellular Signaling Involved in the Induction of Cytoprotective Genes by Some Chemopreventive Phytochemicals. Planta Med. 2008, 74, 1526-1539; Singh, A., et al., RNAi-mediated silencing of nuclear factor erythroid-2-related factor 2 gene expression in non-small cell lung cancer inhibits tumor growth and increases efficacy of chemotherapy. Cancer Res. 2008, 68, 7975-7984.

In addition to Keap1, the activation of protein kinases, such as protein kinase C(PKC), extracellular signal-regulated kinases (ERK), p38 mitogen-activated protein kinase (MAPK), phosphatidylinositol 3-kinase (PI3K) and protein kinase RNA-like endoplasmic reticulum kinase (PERK), has been shown to activate Nrf2. Niture, S. K., et al., Antioxidant-induced modification of INrf2 cysteine 151 and PKC-delta-mediated phosphorylation of Nrf2 serine 40 are both required for stabilization and nuclear translocation of Nrf2 and increased drug resistance. J. Cell Sci. 2009, 122, 4452-4464; Cheng, S. E., et al., Cigarette smoke particle-phase extract induces HO-1 expression in human tracheal smooth muscle cells: role of the c-Src/NADPH oxidase/MAPK/Nrf2 signaling pathway. Free Radical Bioi. Med. 2010, 48, 1410-1422; Nioi, P., et al., Identification of a novel Nrf2-regulated antioxidant response element (ARE) in the mouse NAD(P)H: quinone oxidoreductase 1 gene: reassessment of the ARE consensus sequence. Biochem. J. 2003, 374, 337-348; Chartoumpekis, D., et al., Simvastatin lowers reactive oxygen species level by Nrf2 activation via PI3K/Akt pathway. Biochem. Biophys. Res. Commun. 2010, 396, 463-466; Cullinan, S. B.; Diehl, J. A. PERK-dependent activation of Nrf2 contributes to redox homeostasis and cell survival following endoplasmic reticulum stress. J. Biol. Chem. 2004, 279, 20108-20117.

The Nrf2-regulated genes include almost all of the relevant antioxidants and cytoprotective genes, such as heme oxygenase-1 (HO-1), NAD (P)H:quinone oxidoreductase 1 (NQO1), glutamate-cysteine ligase modifier subunit (GCLM), γ-glutamyl cysteine synthase, glutathione peroxidase (GPx), and several members of the glutathione S-transferase family, Surh, Y. J., et al., Nrf2 as a Master Redox Switch in Turning on the Cellular Signaling Involved in the Induction of Cytoprotective Genes by Some Chemopreventive Phytochemicals. Planta Med. 2008, 74, 1526-1539; Giudice, A., et al., Review of Molecular Mechanisms Involved in the Activation of the Nrf2-ARE Signaling Pathway by Chemopreventive Agents. Transcription Factors: Methods and Protocols. 2010, 37-74; Wan, J. K; Diaz-Sanchez, D. Antioxidant enzyme induction: A new protective approach against the adverse effects of diesel exhaust particles. Inhal. Toxicol. 2007, 19, 177-182; Singh, A., et al., Glutathione peroxidase 2, the major cigarette smoke-inducible isoform of GPX in lungs, is regulated by Nrf2. Am. J. Respir. Cell Mol. Biol. 2006, 35, 639-650; Lin, H. J., et al., Glutathione transferase GSTT1, broccoli, and prevalence of colorectal adenomas. Pharmacogenetics. 2002, 12, 175-179; Kirsch, M.; De Groot, H. NAD(P)H, a directly operating antioxidant? FASEB J. 2001, 15, 1569-1574. Lee, J. S.; Surh, Y. J. Nrf2 as a novel molecular target for chemoprevention. Cancer Lett. 2005, 224, 171-184, that express an ARE in their promoter. Nguyen, T., et al., Regulatory mechanisms controlling gene expression mediated by the antioxidant response element. Annu. Rev. Pharmacool. Toxicol. 2003, 43, 233-260.

Small molecules that activate Nrf2 signaling are being investigated as potential anti-cancer or therapeutic agents. A wide variety of dietary and synthetic compounds that function as potent inducers of ARE-regulated gene expression have been shown to exert chemopreventive activities, e.g., sulforaphane, Thimmulappa, R. K., et al., Identification of Nrf2-regulated genes induced by the chemopreventive agent sulforaphane by oligonucleotide microarray. Cancer Res. 2002, 62, 5196-5203; Dinkova-Kostova, A. T., et al., Direct evidence that sulfhydryl groups of Keap1 are the sensors regulating induction of phase 2 enzymes that protect against carcinogens and oxidants. Proc. Natl. Acad. Sci. U.S.A. 2002, 99, 11908-11913; Yu, R., et al., Role of a mitogen-activated protein kinase pathway in the induction of phase II detoxifying enzymes by chemicals. J. Biol. Chem. 1999, 274, 27545-27552; McMahon, M., et al., Keap1-dependent proteasomal degradation of transcription factor Nrf2 contributes to the negative regulation of antioxidant response element-driven gene expression. J. Biol. Chem. 2003, 278, 21592-21600, dithiolethione, Kwak, M. K., et al., Enhanced expression of the transcription factor Nrf2 by cancer chemopreventive agents: Role of antioxidant response element-like sequences in the nrf2 promoter. Mol. Cell. Biol. 2002, 22, 2883-2892; Kwak, M. K., et al., Modulation of gene expression by cancer chemopreventive dithiolethiones through the Keap1-Nrf2 pathway—Identification of novel gene clusters for cell survival. J. Biol. Chem. 2003, 278, 8135-8145; Kwak, M. K., et al., Role of phase 2 enzyme induction in chemoprotection by dithiolethiones. Mutat. Res. 2001, 480-1, 305-315, curcumin and caffeic acid phenethyl ester (CAPE). Balogun, E., et al., Curcumin activates the haem oxygenase-1 gene via regulation of Nrf2 and the antioxidant-responsive element. Biochem. J. 2003, 371, 887-895. Both curcumin and CAPE bear an α,β-unsaturated ketone moiety and can therefore act as Michael acceptors that are able to modify cysteine thiols present in Keap1.

Chalcones, or 1,2-diphenyl-2-propen-1-ones, are Michael acceptors and constitute an important group of natural products belonging to the flavonoid family. Go, M. L., et al., Chalcones: An update on cytotoxic and chemoprotective properties. Curr. Med. Chem. 2005, 12, 483-499; Dimmock, J. R., et al., Bioactivities of chalcones. Curr. Med. Chem. 1999, 6, 1125-1149. Chalcones have been reported to possess many biological properties including anti-cancer, Xia, Y., et al., Antitumor agents. Part 202: Novel 2′-amino chalcones: Design, synthesis and biological evaluation. Bioorg. Med. Chem. Lett. 2000, 10, 699-701; Bois, F., et al., Halogenated chalcones with high-affinity binding to P-glycoprotein: Potential modulators of multidrug resistance. J. Med. Chem. 1998, 41, 4161-4164, anti-malarial, Liu, M., et al., Antimalarial alkoxylated and hydroxylated chalcones: Structure-activity relationship analysis. J. Med. Chem. 2001, 44, 4443-4452; Dominguez, J. N., et al., Synthesis of quinolinyl chalcones and evaluation of their antimalarial activity. Eur. J. Mad. Chem. 2001, 36, 555-560, anti-inflammatory and antileishmanial, Hsieh, H. K., et al., Synthesis and anti-inflammatory effect of chalcones and related compounds. Pharm. Res. 1998, 15, 39-46; Hsieh, H. K., et al., Synthesis and anti-inflammatory effect of chalcones. J. Pharm. Pharmacol. 2000, 52, 163-171; Herencia, F., et al., Synthesis and anti-inflammatory activity of chalcone derivatives. Bioorg. Med. Chem. Lett. 1998, 8, 1169-1174, anti-tuberclulosis, Lin, Y. M., et al., Chalcones and flavonoids as anti-tuberculosis agents. Biorg. Med. Chem. 2002, 10, 2795-2802, nitric oxide inhibition, Rojas, J., et al., The synthesis and effect of fluorinated chalcone derivatives on nitric oxide production. Bioorg. Med. Chem. Lett. 2002, 12, 1951-1954; Herencia, F., et al., 4-dimethylamino-3′,4′-dimethoxychalcone downregulates iNOS expression and exerts anti-inflammatory effects. Free Radical Biol. Med. 2001, 30, 43-50, anti-mitotic, Ducki, S., et al., Potent antimitotic and cell growth inhibitory properties of substituted chalcones. Bioorg. Med. Chem. Lett. 1998, 8, 1051-1056, analgesic, antipyretic, antioxidant, Maria, K., et al., Synthesis and Anti-Inflammatory Activity of Chalcones and Related Mannich Bases. Med. Chem. 2008, 4, 586-596; Viana, G. S. B., et al., Analgesic and antiinflammatory affects of chalcones isolated from Myracrodruon urundeuva Allemao. Phytomedicine. 2003, 10, 189-195; Cheng, J. H., et al., Synthesis and cytotoxic, anti-inflammatory, and anti-oxidant activities of 2′,5′-dialkoxylchalcones as cancer chemopreventive agents. Biorg. Med. Chem. 2008, 44, 16, 7270-7276; Heidari, M. R., et al., Evaluation of Anti-inflammatory and Analgesic Activity of a Novel Rigid 3,4-Dihydroxy Chalcone in Mice. Natural Compounds and Their Role in Apoptotic Cell Signaling Pathways. 2009, 1171, 399-406, antibacterial, anti-HIV, Cheenpracha, S., et al., Anti-HIV-1 protease activity of compounds from Boesenbergia pandurata. Biorg. Med. Chem. 2006, 14, 1710-1714, antifungal, Svetaz, L., et al., Antifungal chalcones and new caffeic acid esters from Zuccagnia punctata acting against soybean infecting fungi. J. Agric. Food. Chem. 2004, 52, 3297-3300, and antiprotozoal activities. Saavedra, M. J., et al., Antimicrobial activity of phenolics and glucosinolate hydrolysis products and their synergy with streptomycin against pathogenic bacteria. Med. Chem. 2010, 6, 174-83; Aponte, J. C., et al., Synthesis, cytotoxicity, and anti-Trypanosoma cruzi activity of new chalcones. J. Med. Chem. 2008, 51, 6230-6234; Lahtchev, K. L., et al., Antifungal activity of chalcones: A mechanistic study using various yeast strains. Eur. J. Med. Chem. 2008. 43, 2220-2228.

Chalcones also are reported to be gastric protectant, Batovska, D. I.; Todorova, I. T. Trends in utilization of the pharmacological potential of chalcones. Curr Clin Pharmacol. b2010, 5, 1-29, anti-mutagenic, and anti-tumorogenic. Torigoe, T, et al., Anti-mutagenic chalcones-antagonizing the mutagenicity of benzo (A) pyrene on salmonella-typhimurium. Biochem. Biophys. Res. Commun. 1983, 112, 833-842; Lee, S. A., et al., Blockade of Four-Transmembrane L6 Family Member 5 (TM4SF5)-Mediated Tumorigenicity in Hepatocytes by a Synthetic Chalcone Derivative. Hepatology. 2009, 49, 1316-1325; Shibata, S., Anti-tumerigenic chalcones. Stem Cells. 1994, 12, 44-52.

Natural and synthetic chalcones have been reported to possess strong antiproliferative effects in primary and established ovarian cancer cell, Devincenzo, R., et al., Effect of synthetic and naturally-occurring chalcones on ovarian-cancer cell-growth-structure-activity-relationships. Anti-Cancer Drug Des. 1995, 10, 481-490, and in gastric cancer cells; Shibata, S., Anti-tumerigenic chalcones. Stem Cells. 1994, 12, 44-52. Chalcones contain two aromatic rings separated by an α,β-unsaturated ketone. This unique structure is responsible for various activities of these molecules. Go, M. L., et al., Chalcones: An update on cytotoxic and chemoprotective properties. Curr. Med. Chem. 2005, 12, 483-499. The α,β-unsaturated carbonyl entity in chalcones is a soft electrophile, which attracts soft nucleophiles, such as thiols, rather than hard nucleophiles, such as amino and hydroxyl groups. Chalcones are unlikely to react with the amino and hydroxyl groups on nucleic acids and thus would unlikely induce mutagenicity and carcinogenicity commonly associated with alkylating agents used in cancer chemotherapy. Dimmock, J. R., et al., Bioactivities of chalcones. Curr. Med. Chem. 1999, 6, 1125-1149.

The biological potential of chalcones is due to their possible interactions with various proteins related to cell apoptosis and proliferation. Wei, B. L., et al., Synthetic 2′,5′-dimethoxychalcones as G(2)/M arrest-mediated apoptosis-inducing agents and inhibitors of nitric oxide production in rat macrophages. Eur. J. Med. Chem. 2007, 42, 660-668; Takahashi, T., et al., Isoliquiritigenin, a flavonoid from licorice, reduces prostaglandin E-2 and nitric oxide, causes apoptosis, and suppresses aberrant crypt foci development. Cancer Sci. 2004, 95, 448-453.

A number of recent studies have indicated that the anti-inflammatory effect of chalcones is due to the inhibition of the NF-κB pathway, which is mediated by Iκ8 degradation and the phosphorylation of c-Jun N-terminal kinase (JNK) and c-Jun. Lee, J. H., et al., Blockade of nuclear factor-kappa B signaling pathway and anti-inflammatory activity of cardamomin, a chalcone analog from Alpinia conchigera. J. Pharmacol. Exp. Ther. 2006, 316, 271-278; Ban, H. S., et al., Inhibition of lipopolysaccharide-induced expression of inducible nitric oxide synthase and tumor necrosis factor-alpha by 2′-hydroxychalcone derivatives in RAW 264.7 cells. Biochem. Pharmacal. 2004, 67, 1549-1557; Liu, Y. C., et al., Chalcone inhibits the activation of NF-kappa Band STAT3 in endothelial cells via endogenous electrophile. Life Sci. 2007, 80, 1420-1430.

It has been reported that an electrophilic α,β-unsaturated carbonyl moiety on chalcone resulted in the activation of the Nrf2/ARE pathway and the induction of phase II detoxifying enzyme expression. Foresti, R., et al., Differential activation of heme oxygenese-1 by chalcones and rosolic acid in endothelial cells. J. Pharmacol. Exp. Ther. 2005, 312, 686-693; Wu, C. C., et al., Upregulation of endothelial heme oxygenase-1 expression through the activation of the JNK pathway by sublethal concentrations of acrolein. Taxicol. Appl. Pharmacol. 2006, 214, 244-252. This moiety acts as an electrophile and reacts with free sulfhydryl groups of thioredoxin and cysteine residues in proteins. Liu, Y. C., et al., Chalcone inhibits the activation of NF-kappa Band STAT3 in endothelial cells via endogenous electrophile. Life Sci. 2007, 80, 1420-1430; Foresti, R., et al., Differential activation of heme oxygenese-1 by chalcones and rosolic acid in endothelial cells. J. Pharmacol. Exp. Ther. 2005, 312, 686-693; Wu, C. C., et al., Upregulation of endothelial heme oxygenase-1 expression through the activation of the JNK pathway by sublethal concentrations of acrolein. Taxicol. Appl. Pharmacol. 2006, 214, 244-252; Shibata, T., et al., Thioredoxin as a molecular target of cyclopentenone prostaglandins. Free Radical Biol. Med. 2003, 35, 384.

It also has been reported that electrophilic phytochemicals could give rise to thiyl radicals, which also could interact with sulfhydryl residues of intracellular targets, including Nrf2. Heiss, E., et al., Nuclear factor kappa B is a molecular target for sulforaphane-mediated anti-inflammatory mechanisms. J. Biol. Chem. 2001, 276, 32008-32015. These studies demonstrate that the endogenous electrophilic activity, through its α,β-unsaturated carbonyl moiety, is involved in the anti-oxidant and anti-inflammatory properties of chalcone.

A. Chemistry

Chalcones can be readily synthesized by the base-catalyzed Chaisen-Schmidt condensation of an aldehyde and ketone in a polar solvent, for example, ethanol or methanol. The traditional synthesis of chalcones involves the use of strong bases, such as NaOH, Liu, M., et al., Antimalarial alkoxylated and hydroxylated chalcones: Structure-activity relationship analysis. J. Med. Chem. 2001, 44, 4443-4452; Herencia, F., et al., Synthesis and anti-inflammatory activity of chalcone derivatives. Bioorg. Med. Chem. Lett. 1998, 8, 1169-1174; Ducki, S., et al., Potent antimitotic and cell growth inhibitory properties of substituted chalcones. Bioorg. Med. Chem. Lett. 1998, 8, 1051-1056; Micheli, F., et al., A combinatorial approach to [1,5]benzothiazepine derivatives as potential antibacterial agents. J. Comb. Chem. 2001, 3, 224-228, KOH, Lin, H. J., et al., Glutathione transferase GSTT1, broccoli, and prevalence of colorectal adenomas. Pharmacogenetics. 2002, 12, 175-179; Bu, X. Y., et al., A facile synthesis of 6-C-prenylflavanones. Synthesis-Stuttgart. 1997, 1246-1248, Ba(OH)₂, Sinisterra, J. V., et al., An improved procedure for the Claisen-Schmidt reaction. Synthesis-Stuttgart. 1984, 502-504; Alcantara, A. R., et al., Synthesis of 2′-hydroxychalcones and related-compounds in interfacial solid-liquid conditions. Tetrahedron Lett. 1987, 28, 1515-1518, hydrotalcites, Climent, M. J., et al., Activated hydrotalcites as catalysts for the synthesis of chalcones of pharmaceutical interest. J. Catal. 2004, 221, 474-482, and LiHMDS, Daskiewicz, J. B., et al., Organolithium mediated synthesis of prenylchalcones as potential inhibitors of chemoresistance. Tetrahedron Lett. 1999, 40, 7095-7098, calcined NaNO₃/natural phosphate, Sebti, S., et al., Dramatic activity enhancement of natural phosphate catalyst by lithium nitrate. An efficient synthesis of chalcones. Catal. Commun. 2002, 3, 335-339.

Chalcones also can be synthesized by acid-catalyzed aldol condensations, e.g., AICl₃, Calloway, N. O.; Green, L. D. Reactions in the presence of metallic halides 1 beta-unsaturated ketone formation as a side reaction in Friedel-Crafts acylations. J. Am. Chem. Soc. 1937, 59, 809-811, BF₃ or dry HCl, Szell, T.; Sohar, I., New nitrochalcones. Can. J. Chem. 1969, 47, 1254-1258, Zn(bpy)(OAc)₂, Irie, K; Watanabe, K., Aldol condensations with metal(II) complex catalysts. Bull. Chem. Soc. Jpn. 1980, 53, 1366-1371, Cp₂ZrH₂/NiCl₂, Nakano, T., et al., Cross-condensation reactions of cycloalkanones with aldehydes and primary alcohols under the influence of zirconocene complexes. J. Org. Chem. 1987, 52, 2239-2244, and RuCI₃ (for cyclic and acyclic ketones), Iranpoor, N.; Kazemi, F. RuCl₃ catalyses aldol condensations of aldehydes and ketones. Tetrahedron. 1998, 54, 9475-9480. Suzuki coupling also has been employed for the synthesis of chalcone derivatives. Eddarir, S., et al., An efficient synthesis of chalcones based on the Suzuki reaction. Tetrahedron Lett. 2003, 44, 5359-5363.

Several disadvantages of these procedures include long reaction time, high reaction temperature, complex reaction conditions and the use of expensive and non-commercial reagents. Recently, an efficient and facile synthesis of chalcones by condensation of aldehydes and ketones has been reported using LiOH.H₂O as a dual activation catalyst under mild conditions. Bhagat, S., et al. LiOH.H₂O as a novel dual activation catalyst for highly efficient and easy synthesis of 1,3-diaryl-2-propenones by Claisen-Schmidt condensation under mild conditions. J. Mol. Catal. A-Chem. 2006, 244, 20-24.

The presently disclosed subject matter used similar conditions for the synthesis of all chalcone derivatives disclosed herein. In short, the appropriately substituted acetophenone was dissolved in ethanol followed by addition of catalytic amount of LiH.H₂O. The reaction mixture was stirred at room temperature for 15 minutes and the desired substituted benzaldehyde was added. The reaction was carried out at room temperature until completion and the corresponding chalcone derivative (1a-5l) was isolated by crystallization or by silica gel flash chromatography (Table 2). All chalcone derivatives were characterized by ¹H, ¹³C NMR, and HRMS.

TABLE 2
Nrf2 Induction Activity of Chalcone Derivatives

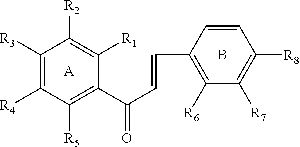

| | | | | | | | Substituents | | | Relative Fold | |
|---|---|---|---|---|---|---|---|---|---|---|---|
| | | Substituents on Ring A | | | | | on Ring B | | | Change^{a} | |
| Entry | Chalcone | R₁ | R₂ | R₃ | R₄ | R₅ | R₆ | R₇ | R₈ | GCLM | NQO1 |
| 1 | 1a | H | H | H | H | H | H | H | H | 0.7 | 1.8 |
| 2 | 1b | OMe | H | H | H | H | H | H | H | 3.6 | 3.2 |
| 3 | 1c | H | OMe | H | H | H | H | H | H | 1.5 | 2.4 |
| 4 | 1d | H | H | OMe | H | H | H | H | H | 1.9 | 2.8 |
| 5 | 1e | OMe | H | OMe | H | H | H | H | H | 2.7 | 2.8 |
| 6 | 1f | OMe | H | H | H | OMe | H | H | H | 2.5 | 1.9 |
| 7 | 1g | OMe | H | H | OMe | H | H | H | H | 3.0 | 2.6 |
| 8 | 1h | H | OMe | OMe | H | H | H | H | H | 2.3 | 2.5 |
| 9 | 1i | H | OMe | H | OMe | H | H | H | H | 3.0 | 2.4 |
| 10 | 1j | H | OMe | OMe | OMe | H | H | H | H | 0.7 | 0.7 |
| 11 | 1k | OMe | OMe | OMe | H | H | H | H | H | 2.4 | 2.4 |
| 12 | 1l | OMe | H | OMe | H | OMe | H | H | H | 3.1 | 1.8 |
| 13 | 2a | H | H | H | H | H | CF₃ | H | H | 5.0 | 5.3 |
| 14 | 2b | OMe | H | H | H | H | CF₃ | H | H | 4.5 | 4.6 |
| 15 | 2c | H | OMe | H | H | H | CF₃ | H | H | 5.6 | 4.3 |
| 16 | 2d | H | H | OMe | H | H | CF₃ | H | H | 5.4 | 4.6 |
| 17 | 2e | OMe | H | OMe | H | H | CF₃ | H | H | 5.4 | 4.5 |
| 18 | 2f | OMe | H | H | H | OMe | CF₃ | H | H | 5.7 | 5.3 |
| 19 | 2g | OMe | H | H | OMe | H | CF₃ | H | H | 4.4 | 2.7 |
| 20 | 2h | H | OMe | OMe | H | H | CF₃ | H | H | 4.3 | 2.7 |
| 21 | 2i | H | OMe | H | OMe | H | CF₃ | H | H | 4.0 | 3.7 |
| 22 | 2j | H | OMe | OMe | OMe | H | CF₃ | H | H | 3.0 | 3.1 |
| 23 | 2k | OMe | OMe | OMe | H | H | CF₃ | H | H | 5.4 | 4.1 |
| 24 | 2l | OMe | H | OMe | H | OMe | CF₃ | H | H | 4.4 | 4.3 |
| 25 | 3a | H | H | H | H | H | H | CF₃ | H | 3.1 | 2.8 |
| 26 | 3b | OMe | H | H | H | H | H | CF₃ | H | 3.9 | 2.9 |
| 27 | 3c | H | OMe | H | H | H | H | CF₃ | H | 4.6 | 3.5 |
| 28 | 3d | H | H | OMe | H | H | H | CF₃ | H | 2.7 | 2.8 |
| 29 | 3e | OMe | H | OMe | H | H | H | CF₃ | H | 4.6 | 4.0 |
| 30 | 3f | OMe | H | H | H | OMe | H | CF₃ | H | 1.8 | 0.6 |
| 31 | 3g | OMe | H | H | OMe | H | H | CF₃ | H | 1.8 | 0.8 |
| 32 | 3h | H | OMe | OMe | H | H | H | CF₃ | H | 4.1 | 2.9 |
| 33 | 3i | H | OMe | H | OMe | H | H | CF₃ | H | 3.8 | 3.9 |
| 34 | 3j | H | OMe | OMe | OMe | H | H | CF₃ | H | 1.7 | 0.8 |
| 35 | 3k | OMe | OMe | OMe | H | H | H | CF₃ | H | 3.6 | 3.2 |
| 36 | 3l | OMe | H | OMe | H | OMe | H | CF₃ | H | 1.4 | 0.7 |
| 37 | 4a | H | H | H | H | H | H | H | CF₃ | 3.31 | 2.9 |
| 38 | 4b | OMe | H | H | H | H | H | H | CF₃ | 4.3 | 5.4 |
| 39 | 4c | H | OMe | H | H | H | H | H | CF₃ | 2.9 | 3.1 |
| 40 | 4d | H | H | OMe | H | H | H | H | CF₃ | 3.5 | 4.6 |
| 41 | 4e | OMe | H | OMe | H | H | H | H | CF₃ | 5.0 | 4.8 |
| 42 | 4f | OMe | H | H | H | OMe | H | H | CF₃ | 1.4 | 0.6 |
| 43 | 4g | OMe | H | H | OMe | H | H | H | CF₃ | 4.1 | 4.0 |
| 44 | 4h | H | OMe | OMe | H | H | H | H | CF₃ | 3.5 | 2.7 |
| 45 | 4i | H | OMe | OMe | OMe | H | H | H | CF₃ | 4.1 | 3.7 |
| 46 | 4j | OMe | OMe | OMe | H | H | H | H | CF₃ | 4.1 | 3.9 |
| 47 | 4k | OMe | H | OMe | H | OMe | H | H | CF₃ | 2.4 | 2.4 |
| 48 | 5a | H | H | H | H | H | NO₂ | H | H | 2.1 | 1.6 |
| 49 | 5b | OMe | H | H | H | H | NO₂ | H | H | 1.9 | 1.4 |
| 50 | 5c | H | OMe | H | H | H | NO₂ | H | H | 2.9 | 2.7 |
| 51 | 5d | H | H | OMe | H | H | NO₂ | H | H | 4.6 | 4.3 |
| 52 | 5e | OMe | H | OMe | H | H | NO₂ | H | H | 2.7 | 3.3. |
| 53 | 5f | OMe | H | H | H | OMe | NO₂ | H | H | 4.1 | 3.7 |
| 54 | 5g | OMe | H | H | OMe | H | NO₂ | H | H | 1.7 | 0.7 |
| 55 | 5h | H | OMe | OMe | H | H | NO₂ | H | H | 2.3 | 2.7 |
| 56 | 5i | H | OMe | H | OMe | H | NO₂ | H | H | 3.4 | 4.1 |
| 57 | 5j | H | OMe | OMe | OMe | H | NO₂ | H | H | 1.2 | 0.7 |
| 58 | 5k | OMe | OMe | OMe | H | H | NO₂ | H | H | 2.9 | 3.3 |
| 59 | 5l | OMe | H | OMe | H | OMe | NO₂ | H | H | 2.6 | 2.0 |

| | | |
|---|---|---|
| DMSO | 1.0 | 1.0 |
| Sulfora- | 2.7 | 3.6 |
| phane | | |

^{a}Data presented are representative of 3 independent experiments.
Values shown are mean ± SD of quadruplicate wells.

B. Biology

1. Potency of Chalcone Derivatives to Activate the Expression of Nrf2-Regulated Cytoprotective Genes in Human Lung Epithelial Cells.

To investigate the potency of the presently disclosed chalcone derivatives to activate Nrf2, the expression of antioxidant genes, GCLM and NADPH-NQO1, two well characterized transcriptional targets of Nrf2, were measured as surrogate markers. By “marker” is meant any protein or polynucleotide having an alteration in expression level or activity that is associated with a disease, disorder, or condition. It has been previously shown that oxidants or small molecule activators of Nrf2 increase GCLM and NQO1 in cells or tissues of wild-type, but not in Nrf2-deficient mice. Osburn, W. O., et al., Genetic or pharmacologic amplification of Nrf2 signaling inhibits acute inflammatory liver injury in mice. Toxicol. Sci. 2008, 104(1), 218-227.

In the presently disclosed subject matter, to screen novel Nrf2 activators, normal human bronchial epithelial cells (Beas-2B) were treated with chalcone derivatives (10 μM) for 16 h and analyzed the expression of GCLM and NQO1 by quantitative RT-PCR (qRT-PCR). Sulforaphane, a well known potent activator of Nrf2, was included as a positive control. Fifty-nine chalcone derivatives that induce the expression of GCLM and NQO01 were identified (Table 2). Concurrent with the gene expression analysis, the cytotoxicity of the chalcone derivatives was determined using the MTT assay. A total of 20 chalcones showed a higher induction of Nrf2-regulated transcriptional targets than the positive control, e.g., sulforaphane (Table 3).

TABLE 3
Cell Viability of Selected Chalcone Derivatives

| | |
|---|---|
| Relative | |
| Fold Change^{a} | % Cell |

| Entry | Compound | GCLM | NQO1 | Viability |
|---|---|---|---|---|
| 1 | DMSO | 1 | 1 | 100 |
| 2 | sulforaphane | 2.7 | 3.6 | 97.3 |
| 3 | 2a | 5 | 5.3 | 144.8 |
| 4 | 2b | 4.5 | 4.6 | 100.9 |
| 5 | 2c | 5.6 | 4.3 | 96.6 |
| 6 | 2d | 5.4 | 4.6 | 96 |
| 7 | 2e | 5.4 | 4.5 | 105 |
| 8 | 2f | 5.7 | 5.3 | 101.1 |
| 9 | 2i | 4 | 3.7 | 87.1 |
| 10 | 2k | 5.4 | 4.1 | 101.6 |
| 11 | 2l | 4.4 | 4.3 | 145.3 |
| 12 | 3c | 4.6 | 3.5 | 95.3 |
| 13 | 3i | 3.8 | 3.9 | 108.2 |
| 14 | 4b | 4.3 | 5.4 | 91 |
| 15 | 4d | 3.5 | 4.6 | 104.1 |
| 16 | 4e | 5 | 4.8 | 92.6 |
| 17 | 4g | 4.1 | 4 | 91.6 |
| 18 | 4j | 4.1 | 3.7 | 100.1 |
| 19 | 5d | 4.6 | 4.3 | 93.2 |
| 20 | 5f | 4.1 | 3.7 | 75.8 |
| 21 | 5i | 3.4 | 4.1 | 90.9 |

2. Preliminary Structure-Activity Relationship:

The structure-activity relationship analysis showed that the chalcone derivatives 1a-1i without any substitution on ring B were not active. The activity of similar derivatives with trifluoromethyl (CF₃) substitution on ring B enhanced the activity dramatically. The position of CF₃ substitution also was crucial for the activity and cytotoxicity of these compounds. In general, the chalcone derivatives with CF₃ substitution at ortho position on ring B were the most active compounds (entries 13-24, Table 2), followed by para (entries 37-47, Table 2), and meta (entries 25-36, Table 2) substitution. Also, the cytotoxicity data show that the ortho CF₃-substituted chalcones were non cytotoxic. This observation indicates that a 4-bond separation between carbonyl and CF₃ can influence the induction activity. With nitro (NO₂) substitution at ortho position on ring B, the activity decreased and the toxicity increased significantly (entries 48-59, Table 2 and entries 19-21, Table 3). Thus, based on these data, only those chalcone derivatives that showed >4-fold induction of GCLM and NQO1 genes and >95% cell viability were selected for further analysis. Based on these criteria, of the 20 compounds that showed potency to increase Nrf2 activity, compounds 2a-f, 2k, and 2l were selected for further analysis.

3. In Vivo Potency of Identified Lead Compounds to Activate Nrf2 Using Mouse Models.

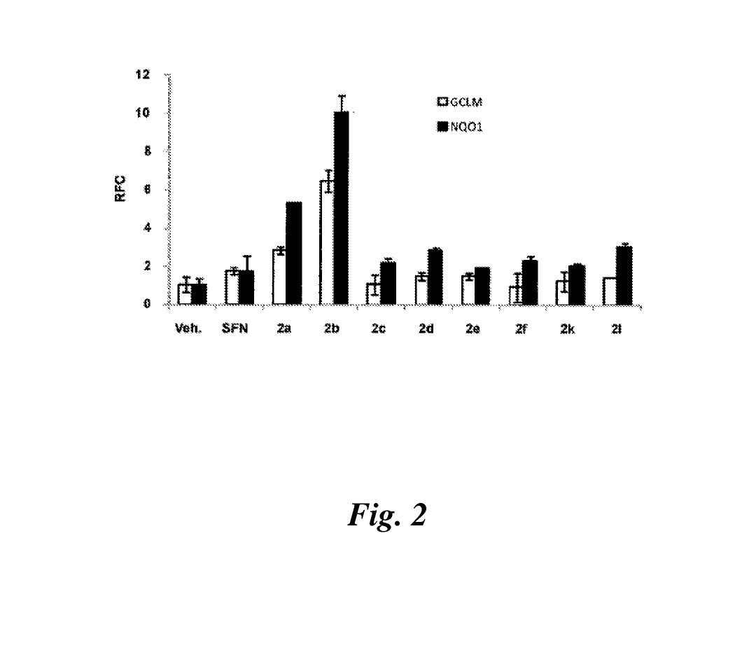

The potency of the 8 lead chalcones identified in the in vitro screening to activate Nrf2 pathway were evaluated in mouse models. First, various formulations to dissolve the compounds were evaluated, and the DCP (10% DMSO+10% Cremophor EL+80% phosphate buffered saline) formulation offered the maximum solubility for delivery of these compounds by oral route. Mice (C57BL/6) were administrated with a single dose of vehicle or test compound(s) or sulforaphane as the positive control at a dose of 50 mg/kg body weight by gavage and small intestines were harvested 24 h later. The expression of Nrf2-regulated genes GCLM and NQO1 was analyzed in the tissue by qRT-PCR. All the eight compounds tested increased the expression of GCLM and NQO1 in small intestine. Compound 2b, however, was the most potent inducer of Nrf2 activity (FIG. 2). The expression of GCLM and NQO1 in the small intestine of mice treated with 2b was 6-fold and 10-fold higher compared to vehicle, respectively. Similarly, the expression of GCLM and NQO1 in the small intestine treated with 2b was 3-fold and 5-fold higher compared to sulforaphane, respectively. Taken together, 2b was selected as the most potent activator of Nrf2 for further studies.

4. Nrf2 is Essential for Induction of Antioxidant Genes by Compound 2b.

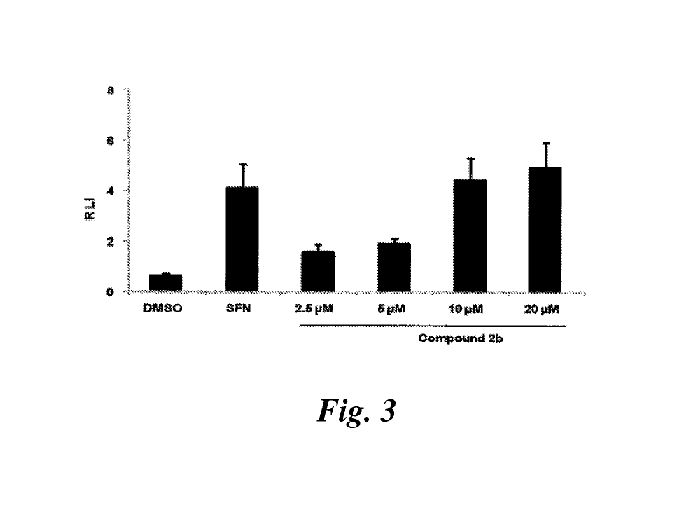

Nrf2 induction by 2b was further characterized by using cell-based assays. Nrf2 increases the expression of NQO1 and GCLM by binding to the ARE present in the promoter region of these genes. Bloom, D., et al., Site-directed mutagenesis of cysteine to serine in the DNA binding region of Nrf2 decreases its capacity to upregulate antioxidant response element-mediated expression and antioxidant induction of NAD(P)H:quinone oxidoreductase1 gene. Oncogene. 2002, 21, 2191-2200. Whether the ARE mediates the transcriptional regulation of NQO1 by 2b also was determined The expression of the luciferase gene under the control of NQO1-ARE sequence was measured using stably transfected Beas-2B cells treated with 2b. The exposure to 2b resulted in a significant concentration-dependent increase in luciferase activity as measured by the chemiluminescence-based assay (FIG. 3). These results implicate the ARE element in the induction of NQO1 gene by compound 2b. The transcriptional activation of antioxidant genes through an ARE is largely dependent upon Nrf2, suggesting that 2b upregulates antioxidant genes via Nrf2 activation.

5. Concentration and Time-Course Studies.

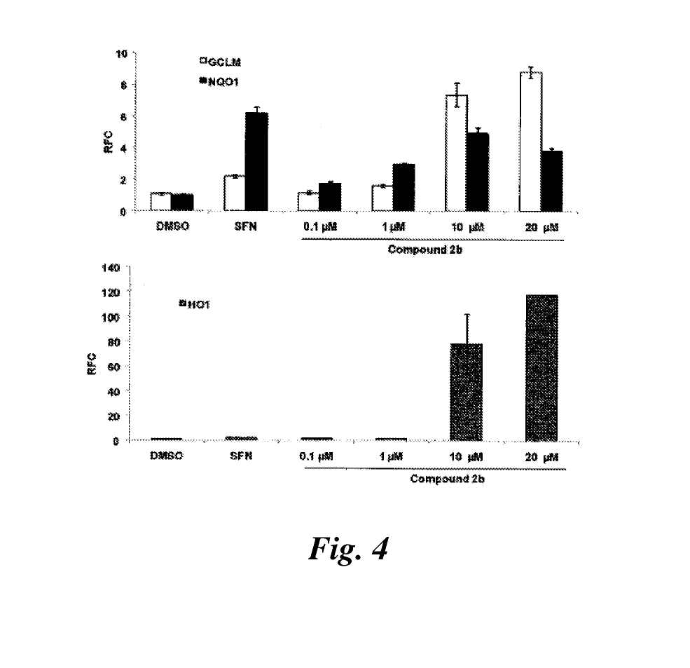

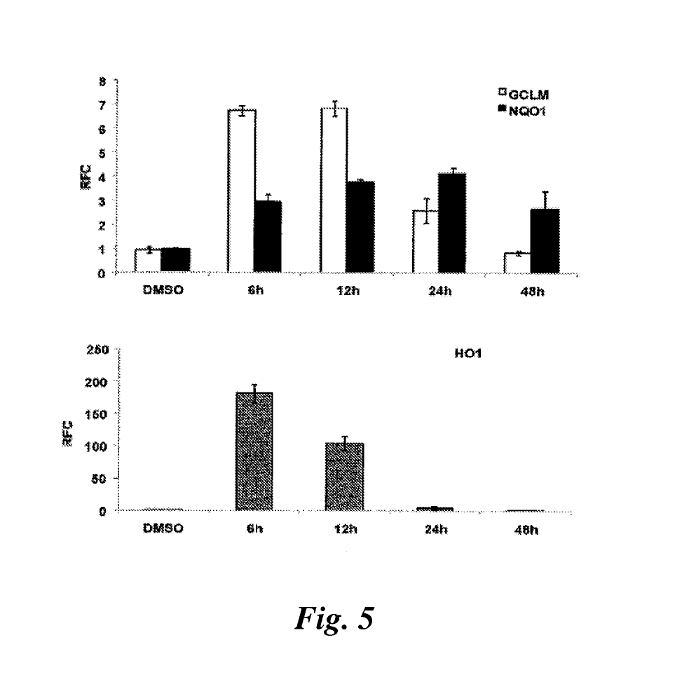

Next, the concentration-dependent effect of 2b on the mRNA levels of Nrf2-driven antioxidant genes, GCLM, NQO1, and HO1 was examined. The expression of these genes at 24 h after treatment was measured with various concentrations (2.5, 5, 10, 20 μM) of 2b in Beas-2B cells. As shown in FIG. 5, compound 2b significantly increased the Nrf2-regulated gene expression in a concentration-dependent manner. There was an approximately 5- and 10-fold increase in the expression of GCLM and NQO1, respectively, at the highest concentration (20 μM) with no cytotoxicity. Interestingly, a dramatic concentration-dependent activation of Nrf2 genes was observed. At 10 μM concentration of 2b, the expression of HO-1 was 6-fold higher compared to sulforaphane (FIG. 4).

For the time-course studies, the expression of antioxidant genes (GCLM, NQO1, and HO-1) was measured at 6, 12, 24 and 48 h after treatment with 2b (10 μM) in Beas-2B cells. The time-course studies showed the highest induction of GCLM (approximately 7-fold) and HO-1 (approximately 150-fold) at 6 h after treatment with 2b (FIG. 5). The expression of GCLM and HO-1 decreased at 6 h after treatment with 2b and was comparable to vehicle by 48 h. The expression of NQO1 was highest at 24 h and remained significantly elevated even at 48 h after treatment with compound 2b compared to vehicle. Taken together, these results suggest that 2b is a potent activator of Nrf2-regulated antioxidant defenses.

6. Activation of Nrf2 by Compound 2b is Independent of ROS Generation.

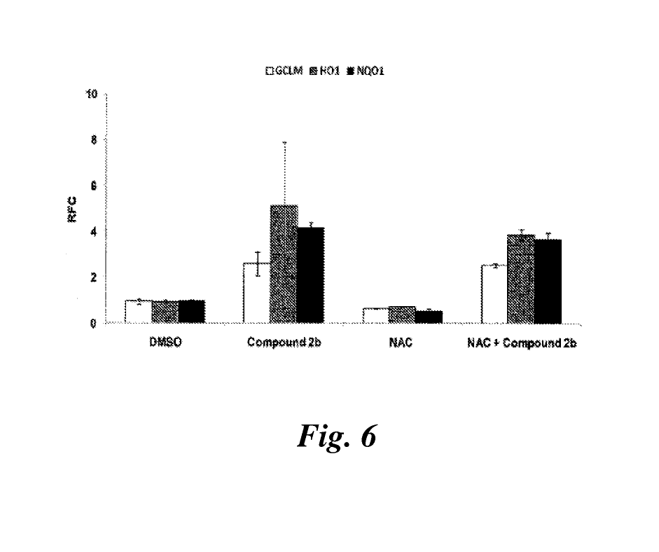

The activation of Nrf2 by various electrophiles and compounds that are Michael acceptors is attributed to changes in ROS production and or redox environment and or direct cysteine modification in Keap1. Nguyen, T., et al., The Nrf2-antioxidant response element signaling pathway and its activation by oxidative stress. J. Biol. Chem. 2009, 284, 13291-13295; McMahon, M., et al., Keap1 perceives stress via three sensors for the endogenous signaling molecules nitric oxide, zinc, and alkenals. Proc. Natl. Acad. Sci. U.S.A. 2010, 107, 18838-18843. Whether 2b activates Nrf2 by generating ROS or redox changes also was examined. Beas-2B cells were co-incubated with compound 2b with or without N-acetyl-cysteine (NAC, 10 mM), and the expression of GCLM, NQO1, and HO1 was measured 24 h later. Compound 2b was found to potentially increase the expression of Nr2-regulated antioxidant genes in the presence of NAC (FIG. 6). NAC alone showed no induction of Nrf2-regulated genes. Taken together, these results suggest that the activation of Nrf2 by 2b is independent of ROS or redox changes.

In summary, novel chalcones, e.g., compound 2b, have been identified as potent activators of Nrf2 signaling pathway after screening a series of chalcone derivatives using cell-based and mouse models.

C. Experimental Section

1. Chemistry

a. General Methods.

TLCs were run on pre-coated Merck silica gel 60F254 plates and observed under UV light. The products were isolated and purified by crystallization or using a Teledyne ISCO Rf Flash chromatography system with hexanes and ethyl acetate as eluents. The ¹H (400 MHz), ¹³C (101 MHz), gCOSY, and gHSQC NMR spectra were taken on a Varian 400-MR spectrophotometer using TMS as an internal standard. Chemical shifts (δ) are expressed in ppm, coupling constants (J) are expressed in Hz, and splitting patterns are described as follows: s=singlet; d=doublet; t=triplet; q=quartet; m=multiplet; dd=doublet of doublets; dt=doublet of triplets; td=triplet of doublets; ddd=doublet of doublet of doublets. For the verification of the product and purity analysis, the LC-MS was taken on an Agilent 1200 series system with an Agilent 6210 Time-Of-Flight (TOF) mass detector using Agilent Eclipse XDB-C-18 column (5 mm, 4.6×150 mm) using a flow rate of 0.9 mL/min and solvent system water (with 0.1% formic acid)/acetonitrile (ACN) (Gradient: 50% ACN@0 min, 80% ACN@7 min, 80% ACN@10 min and 50% ACN@15 min). All chemicals were purchased from Sigma-Aldrich (St. Louis, Mo.) and were used without further purification.

b. General Procedure for Synthesis of Chalcones.

The presently disclosed chalcones were synthesized by the general method depicted in Scheme 1:

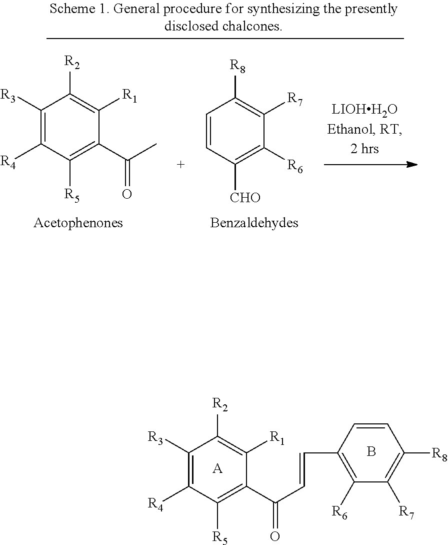

In a 14-mL vial, the substituted acetophenone (1.25 mmol) and lithium hydroxide monohydrate (0.251 mmol) were dissolved in ethanol (5 mL) and the mixture was stirred at room temperature (RT) for 10 min followed by addition of substituted benzaldehyde (1.272 mmol). The reaction mixture was then stirred at RT and monitored by TLC using 25% ethyl acetate/hexanes as the solvent system. The reaction was quenched after 2 hrs by pouring into 50 mL of stiffing ice cold water. If the product precipitated out after quenching with cold water, it was filtered off and crystallized with hot ethanol. In some examples, a sticky mass was observed in the aqueous solution after quenching. In those cases, the product was extracted by ethyl acetate (3×50 mL), dried over sodium sulfate, and concentrated under vacuum. The crude product was purified by flash chromatography using ethyl acetate/hexanes as the solvent system in increasing order of polarity.

### (E)-1-(2-methoxyphenyl)-3-phenylprop-2-en-1-one (1b)

It was obtained as yellow oil in 71% yield. ¹H NMR (400 MHz, DMS) δ=7.76-7.67 (m, 2H), 7.56-7.46 (m, 3H), 7.44-7.37 (m, 4H), 7.18 (d, J=7.9 Hz, 1H), 7.05 (td, J=7.5, 0.9 Hz, 1H), 3.85 (s, 3H). ¹³C NMR (101 MHz, DMSO) δ 192.60, 158.17, 142.97, 135.01, 133.53, 130.94, 129.98, 129.45, 129.23, 128.97, 127.41, 121.01, 112.79, 56.27. LC-MS (ESI-TOF): m/z 239.1072 ([C₁₆H₁₄O₂+H]⁺ calcd. 239.1067). Purity 98.02% (rt 7.21 min).

### (E)-1-(3-methoxyphenyl)-3-phenylprop-2-en-1-one (1c)

It was obtained as yellow oil in 54% yield. ¹H NMR (400 MHz, DMSO) δ 7.95-7.85 (m, 3H), 7.78-7.70 (m, 2H), 7.62-7.58 (m, 1H), 7.49 (d, J=8.0 Hz, 1H), 7.47-7.42 (m, 3H), 7.23 (ddd, J=8.2, 2.6, 0.8 Hz, 1H), 3.84 (s, 3H). ¹³C NMR (101 MHz, DMSO) δ 189.36, 159.99, 144.57, 139.42, 135.07, 131.12, 130.40, 129.42, 129.36, 122.51, 121.52, 119.69, 113.41, 55.83. LC-MS (ESI-TOF): m/z 239.1071 ([C₁₆H₁₄O₂+H]⁺ calcd. 239.1067). Purity 98.52% (rt 7.84 min).

### (E)-1-(4-methoxyphenyl)-3-phenylprop-2-en-1-one (1d)

It was obtained as white solid in 76% yield. ¹H NMR (400 MHz, DMSO) δ 8.16 (d, J=9.0 Hz, 2H), 7.94 (d, J=15.6 Hz, 1H), 7.90-7.83 (m, 2H), 7.69 (d, J=15.6 Hz, 1H), 7.44 (dd, J=5.1, 1.9 Hz, 3H), 7.07 (d, J=9.0 Hz, 2H), 3.85 (s, 3H). ¹³C NMR (101 MHz, DMSO) δ 187.79, 163.68, 143.60, 135.24, 131.39, 130.88, 130.85, 129.33, 129.24, 122.43, 114.48, 56.03. LC-MS (ESI-TOF): m/z 239.1068 ([C₁₆H₁₄O₂+H]⁺ calcd. 239.1067). Purity 100.00% (rt 7.36 min).

### (E)-3-phenyl-1-(2,4-dimethoxyphenyl)prop-2-en-1-one (1e)

It was obtained as yellow oil in 40% yield. ¹H NMR (400 MHz, cdcl₃) δ=7.76 (d, J=8.6 Hz, 1H), 7.68 (d, J=15.8 Hz, 1H), 7.60 (dd, J=7.3, 1.8 Hz, 2H), 7.52 (d, J=15.8 Hz, 1H), 7.43-7.34 (m, 3H), 6.57 (dd, J=8.6, 2.2 Hz, 1H), 6.50 (d, J=2.1 Hz, 1H), 3.91 (s, 3H), 3.87 (s, 3H). ¹³C NMR (101 MHz, DMSO) δ=189.73, 164.44, 160.71, 141.62, 135.32, 132.51, 130.65, 129.43, 128.78, 127.52, 121.79, 106.46, 99.07, 56.42, 56.06. LC-MS (ESI-TOF): m/z 269.1171 ([C₁₇H₁₆O₃+H]⁺ calcd. 269.1172). Purity 96.00% (rt 7.25 min).

### (E)-3-phenyl-1-(2,6-dimethoxyphenyl)prop-2-en-1-one (1f)

It was obtained as white solid in 60% yield. ¹H NMR (400 MHz, DMSO) δ=7.72-7.58 (m, 2H), 7.45-7.31 (m, 4H), 7.17 (d, J=16.2 Hz, 1H), 6.97 (d, J=16.2 Hz, 1H), 6.74 (d, J=8.4 Hz, 2H), 3.70 (s, 6H). ¹³C NMR (101 MHz, DMSO) δ=194.53, 157.29, 144.91, 134.61, 131.35, 131.11, 129.43, 129.04, 129.01, 118.25, 104.86, 56.24. LC-MS (ESI-TOF): m/z 269.1175 ([C₁₇H₁₆O₃+H]⁺ calcd. 269.1172). Purity 100.00% (rt 6.19 min).

### (E)-3-phenyl-1-(2,5-dimethoxyphenyl)prop-2-en-1-one (1g)

It was obtained as yellow oil in 62% yield. ¹H NMR (400 MHz, DMSO) δ=7.76-7.67 (m, 2H), 7.50 (d, J=16.0 Hz, 1H), 7.43 (d, J=2.7 Hz, 3H), 7.40 (d, J=12.2 Hz, 1H), 7.14-7.09 (m, 2H), 7.03 (dd, J=2.6, 0.8 Hz, 1H), 3.80 (s, 3H), 3.73 (s, 3H). ¹³C NMR (101 MHz, DMSO) δ=192.17, 153.47, 152.36, 143.16, 135.00, 130.98, 129.67, 129.46, 128.99, 127.25, 119.02, 114.36, 114.33, 56.80, 56.00. LC-MS (ESI-TOF): m/z 269.1170 ([C₁₇H₁₆O₃+H]⁺ calcd. 269.1172). Purity 98.59% (rt 7.31 min).

### (E)-3-phenyl-1-(3,4-dimethoxyphenyl)prop-2-en-1-one (1h)

It was obtained as yellow oil in 62% yield. ¹H NMR (400 MHz, DMSO) δ 7.95 (d, J=15.6 Hz, 1H), 7.93-7.85 (m, 3H), 7.70 (d, J=15.6 Hz, 1H), 7.60 (d, J=2.0 Hz, 1H), 7.44 (dd, J=1.9, 5.1 Hz, 3H), 7.09 (d, J=8.5 Hz, 1H), 3.86 (s, 3H), 3.84 (s, 3H). ¹³C NMR (101 MHz, DMSO) δ 187.77, 153.67, 149.23, 143.53, 135.25, 130.89, 130.87, 129.32, 129.27, 123.87, 122.36, 111.31, 111.11, 56.23, 56.02. LC-MS (ESI-TOF): m/z 269.1173 ([C₁₇H₁₆O₃+H]⁺ calcd. 269.1172). Purity 97.87% (rt 6.33 min).

### (E)-3-phenyl-1-(3,5-dimethoxyphenyl)prop-2-en-1-one (1i)

It was obtained as yellow oil in 66% yield. ¹H NMR (400 MHz, DMSO) δ 7.94-7.86 (m, 3H). 7.73 (d, J=15.6 Hz, 1H), 7.44 (dd, J=2.6, 3.8 Hz, 3H), 7.25 (d, J=2.3 Hz, 2H), 6.78 (t, J=2.3 Hz, 1H), 3.82 (s, 6H). ¹³C NMR (101 MHz, DMSO) δ 189.16, 161.14, 144.70, 140.05, 135.06, 131.12, 129.48, 129.33, 122.46, 106.71, 105.53, 56.01. LC-MS (ESI-TOF): m/z 269.1176 ([C₁₇H₁₆O₃+H]⁺ calcd. 269.1172). Purity 100.00% (rt 8.09 min).

### (E)-3-phenyl-1-(3,4,5-trimethoxyphenyl)prop-2-en-1-one (1j)

It was obtained as white solid in 68% yield. ¹H NMR (400 MHz, DMSO) δ 7.99-7.85 (m, 3H), 7.73 (d, J=15.5 Hz, 1H), 7.52-7.36 (m, 5H), 3.89 (s, 6H), 3.75 (s, 3H). ¹³C NMR (101 MHz, DMSO) δ 188.34, 153.37, 144.31, 142.44, 135.15, 133.39, 131.03, 129.42, 129.32, 122.35, 106.62, 60.64, 56.67. LC-MS (ESI-TOF): m/z 299.1284 ([C₁₈H₁₈O₄+H]⁺ calcd. 299.1278). Purity 100.00% (rt 6.97 min).

### (E)-3-phenyl-1-(2,3,4.trimethoxyphenyl)prop-2-en-1-one (1k)

It was obtained as white solid in 66% yield. ¹H NMR (400 MHz, DMSO) δ=7.73 (dd, J=6.8, 2.8 Hz, 2H), 7.54 (d, J=15.9 Hz, 1H), 7.48-7.39 (m, 4H), 7.37 (d, J=8.7 Hz, 1H), 6.93 (d, J=8.8 Hz, 1H), 3.86 (s, 3H), 3.83 (s, 3H), 3.77 (s, 3H). ¹³C NMR (101 MHz, DMSO) δ=190.50, 157.16, 153.34, 142.86, 142.07, 135.08, 130.88, 129.47, 128.89, 126.98, 126.58, 125.60, 108.34, 62.16, 60.96, 56.54. LC-MS (ESI-TOF): m/z 299.1275 ([C₁₈H₁₈O₄+H]⁺ calcd. 299.1278). Purity 100.00% (rt 7.19 min).

### (E)-3-phenyl-1-(2,4,6-trimethoxyphenyl)prop-2-en-1-one (1l)

It was obtained as yellow oil in 71% yield. ¹H NMR (400 MHz, DMSO) δ=7.64 (dd, J=6.6, 3.1 Hz, 2H), 7.39 (dd, J=5.1, 1.8 Hz, 3H), 7.19 (d, J=16.1 Hz, 1H), 6.94 (d, J=16.1 Hz, 1H), 6.30 (s, 2H), 3.82 (s, 3H), 3.70 (s, 6H). ¹³C NMR (101 MHz, DMSO) δ=193.70, 162.41, 158.53, 143.97, 134.81, 130.90, 129.46, 129.40, 128.91, 111.45, 91.53, 56.26, 55.91. LC-MS (ESI-TOF): m/z 299.1276 ([C₁₈H₁₈O₄+H]⁺ calcd. 299.1278). Purity 100.00% (rt 6.23 min).

### (E)-1-phenyl-3-(2-(trifluoromethyl)phenyl)prop-2-en-1-one (2a)

It was obtained as yellow solid in 64% yield. ¹H NMR (400 MHz, DMSO) δ=8.35 (d, J=7.8 Hz, 1H), 8.25-8.15 (m, 2H), 8.05 (d, J=15.3 Hz, 1H), 7.98 (dd, J=15.5 Hz, 2.0 Hz, 1H), 7.89-7.76 (m, 2H), 7.76-7.65 (m, 2H), 7.61 (t, J=7.7 Hz, 2H). ¹³C NMR (101 MHz, DMSO) δ=189.33, 138.24 (d, J=1.8 Hz), 137.47, 134.01, 133.42, 133.22 (d, J=1.5 Hz), 130.99, 129.32, 129.26, 129.16, 127.96 (q, J=29.2 Hz), 126.64, 126.62 (q, J=6.0 Hz), 124.61 (q, J=274.5 Hz). LC-MS (ESI-TOF): m/z 277.0833 ([C₁₆H₁₁F₃₀+H]⁺ calcd. 277.0835). Purity 100.00% (rt 6.97 min).

### (E)-1-(2-methoxyphenyl)-3-(2-(trifluoromethyl)phenyl)prop-2-en-1-one (2b)

It was obtained as yellow oil in 72% yield. ¹H NMR (400 MHz, cdcl₃) δ=7.90-7.82 (m, 1H), 7.71 (d, J=7.8 Hz, 1H), 7.63 (d, J=7.8 Hz, 1H), 7.55 (dd, J=7.6 Hz, 1.8, 1H), 7.50 (t, J=7.6 Hz, 1H), 7.45-7.36 (m, 2H), 7.22 (d, J=15.7 Hz, 1H), 6.97 (td, J=7.5 Hz, 0.8 Hz, 1H), 6.92 (d, J=8.4 Hz, 1H), 3.82 (s, 4H). ¹³C NMR (101 MHz, DMSO) δ=192.21, 158.40, 136.83 (d, J=2.1 Hz), 134.05, 133.58, 133.29 (d, J=1.6 Hz), 131.30, 130.82, 130.15, 128.79, 128.58, 127.81 (q, J=29.2 Hz), 126.68 (q, J=5.2 Hz), 124.55 (q, J=274.5 Hz), 121.07, 112.76, 56.31. LC-MS (ESI-TOF): m/z 304.0940 ([C₁₇H₁₃F₃O₂+H]⁺ calcd. 307.0940). Purity 96.40% (rt 8.69 min).

### (E)-1-(3-methoxyphenyl)-3-(2-(trifluoromethyl)phenyl)prop-2-en-1-one (2c)

It was obtained as yellow solid in 26% yield, ¹H NMR (400 MHz, DMSO) δ=8.35 (d, J=7.9 Hz, 1H), 8.06-7.94 (m, 2H), 7.88-7.76 (m, 3H), 7.72-7.63 (m, 2H), 7.52 (t, J=7.9 Hz, 1H), 7.28 (ddd, J=8.2 Hz, 2.7 Hz, 0.8 Hz, 1H), 3.87 (s, 3H). ¹H NMR (400 MHz, cdcl₃) δ=8.13 (d, J=15.6 Hz, 1H), 7.83 (d, J=7.8 Hz, 1H), 7.74 (d, J=7.8 Hz, 1H), 7.60 (dd, J=13.7 Hz, 7.1 Hz, 2H), 7.55-7.47 (m, 2H), 7.41 (dd, J=15.9 Hz, 8.5 Hz, 2H), 7.15 (dd, J=8.2 Hz, 1.9 Hz, 1H), 3.89 (s, 3H). ¹³C NMR (101 MHz, DMSO) δ=189.12, 160.04, 138.92, 138.33 (d, J=2.2 Hz), 133.41, 133.21 (d, J=1.7 Hz), 131.00, 130.47, 129.32, 127.96 (d, J=29.2 Hz), 126.71, 126.62 (d, J=6.0 Hz), 124.62 (d, J=273.5 Hz), 121.69, 120.04, 113.64, 55.86. LC-MS (ESI-TOF): m/z 304.0945 ([C₁₇H₁₃F₃O₂+H]⁺ calcd. 307.0940). Purity 100.00% (rt 9.13 min).

### (E)-1-(4-methoxyphenyl)-3-(2-(trifluoromethyl)phenyl)prop-2-en-1-one (2d)

It was obtained as yellow solid in 37% yield. ¹H NMR (400 MHz, DMSO) δ=8.34 (d, J=7.9 Hz, 1H), 8.24-8.17 (m, 2H), 8.04 (d, J=15.3 Hz, 1H), 7.95 (dd, J=15.4 Hz, 2.2, 1H), 7.87-7.76 (m, 2H), 7.67 (t, J=7.6 Hz, 1H), 7.16-7.07 (m, 2H), 3.89 (s, 3H). ¹³C NMR (101 MHz, DMSO) δ=187.42, 164.00, 137.46 (d, J=2.2 Hz), 133.45 (d, J=1.6 Hz), 133.38, 131.63, 130.78, 130.40, 129.21, 127.87 (d, J=29.2 Hz), 126.67, 126.58 (d, J=6.0 Hz), 124.64 (d, J=274.5 Hz), 114.58, 56.08. LC-MS (ESI-TOF): m/z 304.0944 ([C₁₇H₁₃F₃O₂+H]⁺ calcd. 307.0940). Purity 100.00% (rt 8.75 min).

### (E)-1-(2,4-dimethoxyphenyl)-3-(2-(trifluoromethyl)phenyl)prop-2-en-1-one (2e)

It was obtained as yellow solid in 50% yield. ¹H NMR (400 MHz, DMSO) δ=8.07 (d, J=7.6 Hz, 1H), 7.79 (dt, J=14.8 Hz, 7.9 Hz, 3H), 7.69-7.60 (m, 3H), 6.71 (d, J=2.3 Hz, 1H), 6.67 (dd, J=8.6 Hz, 2.3 Hz, 1H), 3.92 (s, 3H), 3.87 (s, 3H). ¹³C NMR (101 MHz, DMSO) δ=189.18, 164.88, 161.01, 135.47 (d, J=2.0 Hz), 133.64 (d, J=1.3 Hz), 133.59, 132.74, 131.55, 130.55, 128.69, 127.74 (d, J=29.2 Hz), 126.64 (d, J=6.0 Hz), 124.62 (d, J=274.5 Hz) 121.23, 106.69, 99.04, 56.50, 56.13. LC-MS (ESI-TOF): m/z 337.1050 ([C₁₈H₁₅F₃O₃+H]⁺ calcd. 337.1046). Purity 100.00% (rt 8.71 min).

### (E)-1-(2,6-dimethoxyphenyl)-3-(2-(trifluoromethyl)phenyl)prop-2-en-1-one (2f)

It was obtained as white solid in 67% yield. ¹H NMR (400 MHz, DMSO) δ 8.07 (s, 1H), 8.01 (d, J=7.9 Hz, 1H), 7.74 (d, J=7.8 Hz, 1H), 7.61 (t, J=7.8 Hz, 1H), 7.38 (t, J=8.4 Hz, 1H), 7.30 (d, J=16.3 Hz; 1H), 7.14 (d, J=16.2 Hz, 1H), 6.74 (d, J=8.5 Hz, 2H), 3.70 (s, 6H). ¹³C NMR (101 MHz, DMSO) δ 194.45, 157.36, 142.84, 135.92, 132.43, 131.45, 130.69, 130.41, 130.26 (q, J=31.1 Hz), 127.17 (q, J=3.6 Hz), 125.92 (q, J=3.8 Hz), 124.38 (q, J=272.5 Hz), 118.22, 104.90, 56.27. LC-MS (ESI-TOF): m/z 337.1045 ([C₁₈H₁₅F₃O₃+H]⁺ calcd. 337.1046). Purity 100.00% (rt 7.68 min).

### (E)-1-(2,5-dimethoxyphenyl)-3-(2-(trifluoromethyl)phenyl)prop-2-en-1-one (2g)

It was obtained as yellow solid in 80% yield. ¹H NMR (400 MHz, DMSO) δ=8.09 (d, J=7.9 Hz, 1H), 7.83 (d, J=8.0 Hz, 1H), 7.80-7.72 (m, 2H), 7.65 (t, J=7.6 Hz, 1H), 7.51 (d, J=15.7 Hz, 1H), 7.16 (d, J=1.8 Hz, 2H), 7.09 (t, J=1.8 Hz, 1H), 3.83 (s, 3H), 3.76 (s, 3H). ¹³C NMR (101 MHz, DMSO) δ=191.72, 153.50, 152.64, 136.95 (d, J=2.1 Hz), 133.59, 133.28 (d, J=1.7 Hz), 131.14, 130.86, 128.96, 128.79, 127.82 (q, J=29.2 Hz), 126.69 (q, J=16.6 Hz), 124.56 (d, J=273.5 Hz), 119.72, 114.41, 114.33, 56.80, 56.04. LC-MS (ESI-TOF): m/z 337.1049 ([C₁₈H₁₅F₃O₃+H]⁺ calcd. 337.1046). Purity 100.00% (rt 8.76 min).

### (E)-1-(3,4-dimethoxyphenyl)-3-(2-(trifluoromethyl)phenyl)prop-2-en-1-one (2h)

It was obtained as yellow solid in 72% yield. ¹H NMR (400 MHz, DMSO) δ=8.33 (d, J=7.8 Hz, 1H), 8.04 (d, J=15.3 Hz, 1H), 7.96 (dd, J=6.2 Hz, 2.1 Hz, 1H), 7.94 (d, J=2.0 Hz, 1H), 7.84 (d, J=7.8 Hz, 1H), 7.80 (t, J=7.6 Hz, 1H), 7.67 (t, J=7.7 Hz, 1H), 7.63 (d, J=2.0 Hz, 1H), 7.13 (d, J=8.5 Hz, 1H), 3.89 (s, 3H), 3.87 (s, 3H). ¹³C NMR (101 MHz, DMSO) δ=187.44, 154.02, 149.31, 137.42 (d, J=2.0 Hz), 133.49 (d, J=1.0 Hz), 133.37, 130.76, 130.44, 129.26, 127.85 (q, J=29.2 Hz), 126.66, 126.58 (q, J=5.2 Hz), 124.65 (q, J=273.5 Hz), 124.22, 111.40, 111.28, 56.28, 56.07. LC-MS (ESI-TOF): m/z 337.1048 ([C₁₈H₁₅F₃O₃+H]⁺ calcd. 337.1046). Purity 100.00% (rt 7.77 min).

### (E)-1-(3,5-dimethoxyphenyl)-3-(2-(trifluoromethyl)phenyl)prop-2-en-1-one (2i)

It was obtained as yellow oil in 72% yield. ¹H NMR (400 MHz, DMSO) δ=8.36 (d, J=7.9 Hz, 1H), 7.98 (s, 2H), 7.85 (d, J=7.8 Hz, 1H), 7.80 (t, J=7.6 Hz, 1H), 7.68 (t, J=7.6 Hz, 1H), 7.30 (d, J=2.2 Hz, 2H), 6.83 (t, J=2.2 Hz, 1H), 3.85 (s, 6H). ¹H NMR (400 MHz, cdcl₃) δ=8.12 (d, J=13.9 Hz, 1H), 7.82 (d, J=7.8 Hz, 1H), 7.73 (d, J=7.8 Hz, 1H), 7.61 (t, J=7.6 Hz, 1H), 7.51 (t, J=7.7 Hz, 1H), 7.35 (d, J=15.6 Hz, 1H), 7.14 (d, J=2.2 Hz, 2H), 6.69 (t, J=2.1 Hz, 1H), 3.86 (s, 7H). ¹³C NMR (101 MHz, DMSO) δ=188.97, 161.20, 139.53, 138.45 (d, J=2.2 Hz), 133.39, 133.18 (d, J=1.6 Hz), 131.00, 129.40, 127.96 (q, J=29.2 Hz), 126.67, 129.60 (q, J=5.2 Hz), 124.62 (q, J=273.5 Hz), 106.93, 105.88, 56.05. LC-MS (ESI-TOF): m/z 337.1047 ([C₁₈H₁₅F₃O₃+H]⁺ calcd. 337.1046). Purity 100.00% (rt 9.37 min).

### (E)-1-(3,4,5,S.trimethoxyphenyl)-3-(2-(trifluoromethyl)phenyl)prop-2-en-1-one (2j)

It was obtained as yellow solid in 80% yield. ¹H NMR (400 MHz, DMSO) δ=8.31 (d, J=7.8 Hz, 1H), 8.02 (d, J=15.2 Hz, 1H), 7.95 (dd, J=15.4 Hz, 2.1 Hz, 1H), 7.83 (d, J=8.0 Hz, 1H), 7.78 (d, J=7.7 Hz, 1H), 7.66 (t, J=7.6 Hz, 1H), 7.44 (s, 2H), 3.88 (s, 6H), 3.76 (s, 3H). ¹³C NMR (101 MHz, DMSO) δ=188.07, 153.41, 142.77, 138.14 (d, J=2.0), 133.37, 132.86, 130.91, 129.40, 127.77 (q, J=29.2 Hz), 126.61, 126.61 (q, J=5.0 Hz), 124.63 (q, J=274.5 Hz), 106.86, 60.66, 56.68. LC-MS (ESI-TOF): m/z 367.1160 ([C₁₉H₁₇F₃O₄+H]⁺ calcd. 367.1152). Purity 100.00% (rt 8.38 min).

### (E)-1-(2,3,4-trimethoxyphenyl)-3-(2-(trifluoromethyl)phenyl)prop-2-en-1-one (2k)

It was obtained as yellow solid in 58% yield. ¹H NMR (400 MHz, DMSO) δ=8.10 (d, J=7.7 Hz, 1H), 7.80 (dt, J=22.4, 7.7 Hz, 3H), 7.66 (t, J=7.6 Hz, 1H), 7.56 (d, J=15.5 Hz, 1H), 7.46 (d, J=8.8 Hz, 1H), 6.97 (d, J=8.9 Hz, 1H), 3.89 (s, 3H), 3.86 (s, 3H), 3.80 (s, 3H). ¹³C NMR (101 MHz, DMSO) δ=189.74, 157.66, 153.66, 142.08, 136.44 (d, J=2.1 Hz), 133.60, 133.46 (d, J=2.0 Hz), 131.02, 130.73, 128.71, 127.78 (d, J=29.2 Hz), 126.66 (d, J=5.0 Hz), 125.95 (d, J=7.0 Hz), 124.59 (d, J=274.5 Hz), 108.48, 62.21, 60.98, 56.60. LC-MS (ESI-TOF): m/z 367.1152 ([C₁₉H₁₇F₃O₄+H]⁺ calcd. 367.1152). Purity 96.17% (rt 8.70 min).

### (E)-1-(2,4,6-trimethoxyphenyl)-3-(2-(trifluoromethyl)phenyl)prop-2-en-1-one (2l)

It was obtained as yellow solid in 72% yield. ¹H NMR (400 MHz, DMSO) δ=8.04 (d, J=7.8 Hz, 1H), 7.77 (d, J=7.7 Hz, 1H), 7.71 (t, J=7.5 Hz, 1H), 7.60 (t, J=7.6 Hz, 1H), 7.48 (dd, J=15.9, 2.2 Hz, 1H), 7.01 (d, J=15.8 Hz, 1H), 6.30 (s, 2H), 3.82 (s, 3H), 3.70 (s, 6H). ¹³C NMR (101 MHz, DMSO) δ=193.37, 162.78, 162.78, 158.71, 138.33 (d, J=2.2 Hz), 133.51, 133.07, 130.80, 128.83, 127.58 (q, J=29.2 Hz), 126.61 (q, J=6.0 Hz); 124.46 (q, J=274.5 Hz), 110.73, 91.34, 56.23, 55.96. LC-MS (ESI-TOF): m/z 367.1157 ([C₁₉H₁₇F₃O₄+H]⁺ calcd. 367.1152). Purity 96.17% (rt 7.58 min).

### (E)-1-phenyl-3-(3-(trifluoromethyl)phenyl)prop-2-en-1-one (3a)

It was obtained as white solid in 68% yield. ¹H NMR (400 MHz, DMSO) δ 8.36 (s, 1H), 8.26-8.18 (m, 3H), 8.15 (d, J=15.7 Hz, 1H), 7.89-7.78 (m, 2H), 7.75-7.67 (m, 2H), 7.64-7.57 (m, 2H). ¹³C NMR (101 MHz, DMSO) δ 189.51, 142.61, 137.72, 136.28, 133.83, 133.36, 130.38, 130.26 (q, J=28.1 Hz), 127.14 (q, J=3.8 Hz), 125.62 (q, J=3.7 Hz), 124.15 (q, J=272.5 Hz), 124.38. LC-MS (ESI-TOF): m/z 277.0840 ([C₁₆H₁₁F₃O+H]⁺ calcd. 277.0835). Purity 100.00% (rt 9.20 min).

### (E)-1-(2-methoxyphenyl)-3-(3-(trifluoromethyl)phenyl)prop-2-en-1-one (3b)

It was obtained as yellow oil in 45% yield. ¹H NMR (400 MHz, DMSO) δ 8.57 (t, J=1.9 Hz, 1H), 8.29-8.19 (m, 2H), 7.73 (t, J=8.0 Hz, 1H), 7.69-7.61 (m, 2H), 7.61-7.57 (m, 1H), 7.57-7.52 (m, 1H), 7.22 (d, J=7.9 Hz, 1H), 7.08 (td, J=7.5, 0.9 Hz, 1H), 3.89 (s, 3H). LC-MS (ESI-TOF): m/z 304.0941 ([C₁₇H₁₃F₃O₂+H]⁺t calcd. 307.0940). Purity 96.00% (rt 8.88 min).

### (E)-1-(3-methoxyphenyl)-3-(3-(trifluoromethyl)phenyl)prop-2-en-1-one (3c)

It was obtained as white solid in 21% yield. ¹H NMR (400 MHz, DMSO) δ 8.35 (s, 1H), 8.21 (d, J=7.8 Hz, 1H), 8.11 (d, J=15.7 Hz, 1H), 7.87-7.77 (m, 3H), 7.70 (t, J=7.78 Hz, 1H), 7.66 (dd, J=1.6, 2.5 Hz, 1H), 7.52 (t, J=7.94 Hz, 1H), 7.27 (ddd, J=0.8, 2.6, 8.2 Hz, 1H), 3.87 (s, 3H). ¹³C NMR (101 MHz, DMSO) δ 189.28, 160.03, 142.68, 139.18, 136.27, 133.32, 130.41, 130.36, 130.25 (q, J=31.2 Hz), 127.15 (q, J=3.7 Hz), 125.72 (q, J=3.7 Hz), 124.50 (q, J=272.5 Hz), 124.46, 121.70, 119.76, 113.68, 55.85. LC-MS (ESI-TOF): m/z 304.0945 ([C₁₇H₁₃F₃O₂+H]⁺ calcd. 307.0940). Purity 100.00% (rt 9.35 min).

### (E)-1-(4-methoxyphenyl)-3-(3-(trifluoromethyl)phenyl)prop-2-en-1-one (3d)

It was obtained as white solid in 59% yield. ¹H NMR (400 MHz, DMSO) δ 8.33 (s, 1H), 8.24-8.20 (m, 2H), 8.18 (d, J=7.8 Hz, 1H), 8.13 (d, J=15.7 Hz, 1H), 7.82-7.74 (m, 2H), 7.69 (t, J=7.8 Hz, 1H), 7.14-7.07 (m, 2H), 3.88 (s, 3H). ¹³C NMR (101 MHz, DMSO) δ 187.69, 163.85, 141.74, 136.45, 133.23, 131.57, 130.67, 130.34, 130.24 (q, J=32.7 Hz), 126.93 (q, J=3.8 Hz), 125.49 (q, J=3.7 Hz), 124.52 (q, J=272.5 Hz), 124.45, 114.49, 56.05. LC-MS (ESI-TOF): m/z 304.0951 ([C₁₇H₁₃F₃O₂+H]⁺ calcd. 307.0940). Purity 100.00% (rt 8.99 min).

### (E)-1-(2,4-dimethoxyphenyl)-3-(3-(trifluoromethyl)phenyl)prop-2-en-1-one (3e)

It was obtained as white solid in 52% yield. ¹H NMR (400 MHz, DMSO) δ 8.11-8.03 (m, 2H), 7.77 (d, J=7.8 Hz, 1H), 7.72-7.58 (m, 4H), 6.70 (d, J=2.3 Hz, 1H), 6.66 (dd, J=2.3, 8.6 Hz, 1H), 3.90 (s, 3H), 3.86 (s, 3H). ¹³C NMR (101 MHz, d₂o) δ 189.57, 164.52, 160.77, 139.58, 136.52, 132.49, 131.97, 130.39, 130.18 (q, J=31.2 Hz), 129.38, 126.69 (q, J=3.7 Hz), 125.52 (q, J=3.8 Hz, H), 124.37 (q, J=272.5 Hz), 121.55, 106.44, 99.03, 56.38, 56.02. LC-MS (ESI-TOF): m/z 337.1044 ([C₁₈H₁₅F₃O₃+H]⁺ calcd. 337.1046). Purity 100.00% (rt 8.93 min).

### (E)-1-(2,6-dimethoxyphenyl)-3-(3-(trifluoromethyl)phenyl)prop-2-en-1-one (3f)

It was obtained as light yellow solid in 72% yield. ¹H NMR (400 MHz, DMSO) δ=8.07 (d, J=7.8 Hz, 1H), 7.76 (d, J=7.8, 1H), 7.71 (dd, J=11.4 Hz, 4.0 Hz, 1H), 7.61 (t, J=7.6 Hz, 1H), 7.45 (d, J=2.1 Hz, 1H), 7.40 (dd, J=11.1 Hz, 5.7 Hz, 1H), 7.02 (d, J=15.9 Hz, 1H), 6.75 (d, J=8.4 Hz, 2H), 3.70 (s, 6H). ¹³C NMR (101 MHz, DMSO) δ=194.52, 157.33, 139.62 (d, J=2.2 Hz), 133.52, 132.81 (d, J=1.6 Hz), 132.57, 131.77, 131.02, 128.90, 127.59 (q, J=30.2 Hz), 126.64 (q, J=5.7 Hz), 124.38 (q, J==274.5 Hz), 117.44, 104.69, 56.22. LC-MS (ESI-TOF): m/z 337.1050 ([C₁₈H₁₅F₃O₃+H]⁺ calcd. 337.1046). Purity 98.65% (rt 7.83 min).

### (E)-1-(2,5-dimethoxyphenyl)-3-(3-(trifluoromethyl)phenyl)prop-2-en-1-one (3g)

It was obtained as yellow oil in 71% yield. ¹H NMR (400 MHz, DMSO) δ 8.12 (s, 1H), 8.08 (d, J=7.8 Hz, 1H), 7.79 (d, J=7.8 Hz, 1H), 7.67 (t, J=7.8 Hz, 1H), 7.62 (d, J=16.1 Hz, 1H), 7.55 (dd, J=0.9, 16.0 Hz, 1H), 7.18-7.12 (m, 2H), 7.09-7.03 (m, 1H), 3.82 (d, J=0.8 Hz, 3H), 3.76 (d, J=0.9 Hz, 3H). ¹³C NMR (101 MHz, DMSO) δ 192.15, 153.49, 152.47, 141.17, 136.27, 132.29, 130.48, 130.27 (q, J=31.2 Hz), 129.50, 129.14, 127.06 (q, J=3.7 Hz), 125.83 (q, J=3.3 Hz), 124.42 (d, J=272.5 Hz), 119.20, 114.42, 114.37, 56.84, 56.04. LC-MS (ESI-TOF): m/z 337.1048 ([C₁₈H₁₅F₃O₃+H]⁺ calcd. 337.1046). Purity 96.60% (rt 8.96 min).

### (E)-1-(3,4-dimethoxyphenyl)-3-(3-(trifluoromethyl)phenyl)prop-2-en-1-one (3h)

It was obtained as white solid in 64% yield. ¹H NMR (400 MHz, d₂o) δ 8.11 (dd, J=11.7, 8.7 Hz, 3H), 7.98-7.91 (m, 1H), 7.79 (dd, J=19.6, 12.0 Hz, 3H), 7.63 (d, J=1.9 Hz, 1H), 7.13 (d, J=8.5 Hz, 1H), 3.89 (s, 3H), 3.87 (s, 3H). ¹³C NMR (101 MHz, d₂o) δ 187.59, 153.86, 149.24, 141.43, 139.26 (d, J=1.3 Hz), 130.59, 130.23 (d, J=31.2 Hz), 129.74, 126.02 (q, J=3.7 Hz), 125.06, 124.45 (d, J=272.5 Hz), 124.08, 111.29, 111.13, 56.21, 56.00. LC-MS (ESI-TOF): m/z 337.1041 ([C₁₈H₁₅F₃O₃+H]⁺ calcd. 337.1046). Purity 98.47% (rt 8.03 min).

### (E)-1-(3,5-dimethoxyphenyl)-3-(3-(trifluoromethyl)phenyl)prop-2-en-1-one (3i)

It was obtained as light yellow solid in 72% yield. ¹H NMR (400 MHz, DMSO) δ 8.34 (s, 1H), 8.22 (d, J=7.S Hz, 1H), 8.08 (d, J=15.7 Hz, 1H), 7.88-7.77 (m, 2H), 7.70 (t, J=7.8 Hz, 1H), 7.32 (d, J=2.3 Hz, 2H), 6.83 (t, J=2.2 Hz, 1H), 3.85 (s, 6H). ¹³C NMR (101 MHz, DMSO) δ 189.08, 161.18, 142.83, 139.77, 136.24, 133.33, 130.33, 130.24 (q, J=32.2 Hz), 127.17 (q, J=3.6 Hz), 125.85 (q, J=3.7 Hz), 124.50 (q, J=273.5 Hz), 124.36, 106.98, 105.52, 56.03. LC-MS (ESI-TOF): m/z 337.1049 ([C₁₈H₁₅F₃O₃+H]⁺ calcd. 337.1046). Purity 100.00% (rt 9.55 min).

### (E)-1-(3,4,5-trimethoxyphenyl)-3-(3-(trifluoromethyl)phenyl)prop-2-en-1-one (3j)

It was obtained as white solid in 60% yield. ¹H NMR (400 MHz, DMSO) δ 8.28-8.19 (m, 2H), 8.06 (d, J=15.7 Hz, 1H), 7.86-7.75 (m, 2H), 7.69 (t, J=7.8 Hz, 1H), 7.44 (s, 2H), 3.89 (s, 6H), 3.76 (s, 3H). ¹³C NMR (101 MHz, DMSO) δ 188.34, 153.39, 142.69, 142.48, 136.33, 133.16, 132.95, 130.33, 130.24 (q, J=32.2 Hz), 127.13 (q, J=3.7 Hz), 126.02 (q, J=3.7 Hz), 124.49 (q, J=272.5 Hz), 124.34, 106.90, 60.66, 56.75. LC-MS (ESI-TOF): m/z 367.1155 ([C₁₉H₁₇F₃O₄+H]⁺ calcd. 367.1152). Purity 96.17% (rt 8.61 min).

### (E)-1-(2,3,4-trimethoxyphenyl)-3-(3-(trifluoromethyl)phenyl)prop-2-en-1-one (3k)

It was obtained as yellow oil in 46% yield. ¹H NMR (400 MHz, DMSO) δ 8.13 (s, 1H), 8.09 (d, J=7.8 Hz, 1H), 7.78 (d, J=7.7 Hz, 1H); 7.71-7.56 (m, 3H), 7.42 (d, J=8.7 Hz, 1H), 6.96 (d, J=8.9 Hz, 1H), 3.89 (s, 3H), 3.85 (s, 3H), 3.80 (s, 3H). ¹³C NMR (101 MHz, DMSO) δ 190.46, 157.30, 153.41, 142.11, 140.87, 136.36, 132.23, 130.49, 130.27 (q, J=31.2 Hz), 128.92, 126.96 (q, J=3.7 Hz), 126.42, 125.69, 125.66 (q, J=5.0 Hz), 124.43 (q, J=272.5 Hz), 108.33, 62.14, 60.97, 56.57. LC-MS (ESI-TOF): m/z 367.1157 ([C₁₉H₁₇F₃O₄+H]⁺ calcd. 367.1152). Purity 97.99% (rt 8.83 min).

### (E)-1-(2,4,6-trimethoxyphenyl)-3-(3-(trifluoromethyl)phenyl)prop-2-en-1-one (3l)

It was obtained as light yellow solid in 69% yield. ¹H NMR (400 MHz, DMSO) δ 8.05 (s, 1H), 8.00 (d, J=7.8 Hz, 1H), 7.73 (d, J=7.8 Hz, 1H), 7.61 (t, J=7.8 Hz, 1H), 7.32 (d, J=16.2 Hz, 1H), 7.11 (d, J=16.2 Hz, 1H), 6.30 (s, 2H), 3.82 (s, 3H), 3.70 (s, 6H). ¹³C NMR (101 MHz, DMSO) δ 193.58, 162.54, 158.63, 141.94, 136.11, 132.29, 130.39, 130.24 (q, J=30.2 Hz), 127.00 (d, J=4.0 Hz), 125.81 (d, J=4.0 Hz), 124.40 (q, J=31 272.5 Hz), 111.35, 91.55, 56.27, 55.93. LC-MS (ESI-TOF): m/z 367.1154 ([C₁₉H₁₇F₃O₄+H]⁺ calcd. 367.1152). Purity 100.00% (rt 7.86 min).

### (E)-1-phenyl-3-(4-(trifluoromethyl)phenyl)prop-2-en-1-one (4a)

It was obtained as white solid in 59% yield. ¹H NMR (400 MHz, DMSO) δ 8.20-8.11 (m, 3H), 8.08 (d, J=15.7 Hz, 2H), 7.83-7.75 (m, 3H), 7.72-7.65 (m, 1H), 7.62-7.54 (m, 2H). ¹³C NMR (101 MHz, DMSO) δ89.53, 142.43, 139.13 (d, J=1.3 Hz), 137.68, 133.87, 130.49 (q, J=32.2 Hz), 129.91, 129.30, 129.11, 126.14 (q, J=3.8 Hz), 125.13, 124.44 (q, J=272.5 Hz). LC-MS (ESI-TOF): m/z 277.0834 ([C₁₆H₁₁F₃O+H]⁺ calcd, 277.0835). Purity 100.00% (rt 9.35 min).

### (E)-1-(2-methoxyphenyl)-3-(4-(trifluoromethyl)phenyl)prop-2-en-1-one (4b)

It was obtained as yellow oil in 79% yield. ¹H NMR (400 MHz, DMSO) δ 7.97 (d, J=8.1 Hz, 2H), 7.79 (d, J=8.2 Hz, 2H), 7.72 (d, J=8.2 Hz, 1H), 7.61-7.48 (m, 6H), 7.22 (d, J=8.0 Hz, 1H), 7.08 (td, J=7.5, 0.9 Hz, 1H), 3.88 (s, 3H), 3.86 (s, 2H). LC-MS (ESI-TOF): m/z 304.0948 ([C₁₇H₁₃F₃O₂+H]⁺ calcd. 307.0940). Purity 94.00% (rt 9.09 min).

### (E)-1-(3-methoxyphenyl)-4-(4-(trifluoromethyl)phenyl)prop-2-en-1-one (4c)

It was obtained as white solid in 61% yield. ¹H NMR (400 MHz, DMSO) δ 8.14 (d, J=8.1 Hz, 2H), 8.07 (d, J=15.7 Hz, 1H), 7.85-7.80 (m, 3H), 7.79 (d, J=4.3 Hz, 1H), 7.67-7.62 (m, 1H), 7.52 (t, J=7.9 Hz, 1H), 7.27 (dd, J=8.2, 2.6 Hz, 1H), 3.87 (s, 3H). ¹³C NMR (101 MHz, DMSO) δ 189.29, 160.04, 142.49, 139.13, 13.50 (d, J=31.2 Hz), 130.45, 129.95, 126.12 (q, J=3.7 Hz), 124.5 (d, J=272.46 Hz), 125.20, 121.65, 119.90, 113.58, 55.86. LC-MS (ESI-TOF): m/z 304.0943 ([C₁₇H₁₃F₃O₂+H]⁺ calcd. 307.0940). Purity 100.00% (rt 9.52 min).

### (E)-1-(4-methoxyphenyl)-3-(4-(trifluoromethyl)phenyl)prop-2-en-1-one (4d)

It was obtained as white solid in 56% yield. ¹H NMR (400 MHz, DMSO) δ 8.23-8.01 (m, 5H), 7.76 (dd, J=21.4, 11.6 Hz, 3H), 7.08 (d, J=8.2 Hz, 2H), 3.86 (s, 3H). ¹³C NMR (101 MHz, DMSO) δ 187.70, 163.89, 141.58, 139.30, 131.55, 130.60, 130.32 (d, J=32.2 Hz), 129.78, 126.11 (dd, J=3.8 Hz), 125.18, 124.5 (d, J=272.5 Hz), 114.55, 56.06. LC-MS (ESI-TOF): m/z 304.0940 ([C₁₇H₁₃F₃O₂+H]⁺ calcd. 307.0940). Purity 100.00% (rt 9.13 min).

### (E)-1-(2,4-dimethoxyphenyl)-3-(4-(trifluoromethyl)phenyl)prop-2-en-1-one (4e)

It was obtained as off white solid in 40% yield. ¹H NMR (400 MHz, DMSO) δ 7.92 (d, J=7.4 Hz, 2H), 7.76 (d, J=7.5 Hz, 2H), 7.70-7.50 (m, 3H), 6.74-6.57 (m, 2H), 3.89 (s, 3H), 3.84 (s, 3H). ¹³C NMR (101 MHz, DMSO) δ 189.45, 164.74, 160.94, 139.41, 132.65, 130.12) q, J=32.2 Hz), 130.10, 129.33, 126.21 (dd, J=3.5 Hz), 124.50 (d, J=272.5 Hz), 121.48, 106.62, 99.07, 56.48, 56.10. LC-MS (ESI-TOF): m/z 337.1046 ([C₁₈H₁₅F₃O₃+H]⁺ calcd. 337.1046). Purity 96.35% (rt 9.13 min).

### (E)-1-(2,6-dimethoxyphenyl)-3-(4-(trifluoromethyl)phenyl)prop-2-en-1-one (4f)

It was obtained as light yellow solid in 64% yield. ¹H NMR (400 MHz, DMSO) δ 7.92 (d, J=8.3 Hz, 2H), 7.75 (d, J=8.2 Hz, 2H), 7.41 (t, J=8.4 Hz, 1H), 7.29 (d, J=16.2 Hz, 1H), 7.13 (d, J=16.2 Hz, 1H), 6.77 (d, J=8.5 Hz, 2H), 3.73 (s, 6H). ¹³C NMR (101 153.39, 149.18, 142.64, 139.01, 134.20, 132.84, 131.45, 130.35, 130.12, 126.80, 125.16, 106.80, 60.66, 56.65. LC-MS (ESI-TOF): m/z 344.1130 ([C₁₈H₁₇NO₆+H]⁺ calcd. 344.1129). Purity 99.00% (rt 6.25 min).

### (E)-1-(2,3,4-trimethoxyphenyl)-3-(2-nitrophenyl)prop-2-en-1-one (5k)

It was obtained as light white solid in 32% yield. ¹H NMR (400 MHz, DMSO) δ=8.07 (d, J=8.0 Hz, 1H), 7.97 (d, J=7.6 Hz, 1H), 7.85-7.75 (m, 2H), 7.67 (t, J=7.7 Hz, 1H), 7.43 (dd, J=12.2 Hz, 3.2 Hz, 2H), 6.95 (d, J=8.8 Hz, 1H), 3.87 (s, 3H), 3.84 (s, 3H), 3.77 (s, 3H). LC-MS (ESI-TOF): m/z 344.1127 ([C₁₈H₁₇NO₆+H]⁺t calcd. 344.1129). Purity 100.00% (rt 6.52 min).

### (E)-1-(2,4,6-trimethoxyphenyl)-3-(2-nitrophenyl)prop-2-en.1-one (5l)

It was obtained as yellow solid in 61% yield. ¹H NMR (400 MHz, DMSO) δ=8.03 (dd, J=8.1 Hz, 1.0, 1H), 7.93 (d, J=7.7 Hz, 1H), 7.75 (t, J=7.6 Hz, 1H), 7.64 (t, J=7.8 Hz, 1H), 7.53 (d, J=15.9 Hz, 1H), 6.92 (d, J=15.9 Hz, 1H), 6.30 (s, 2H), 3.82 (s, 3H), 3.72 (s, 6H). ¹³C NMR (101 MHz, DMSO) δ=193.25, 162.76, 158.80, 148.78, 138.79, 134.30, 133.17, 131.33, 130.06, 129.61, 125.18, 110.82, 91.42, 56.26, 55.94. LC-MS (ESI-TOF): m/z 344.1141 ([C₁₈H₁₇NO₆+H]⁺ calcd. 344.1129). Purity 100.00% (rt 5.77 min).

2. Biology

a. Cell Culture and Treatment.

Human bronchial epithelial (Beas-2B) cells were cultured in DMEM:F12 (pH 7.4) supplemented with 10% (v/v) FBS, 100 mg/L gentamicin and genetisin. Beas-2B cells were grown in 48-well plates for 24 h and then treated with a series of chalcone derivatives dissolved in DMSO for various time points. The concentration of DMSO did not exceed 0.1%. RNA was isolated and gene expression was measured after 16 h.

b. Cell Viability Assay.

The cytotoxicity of the presently disclosed chalcone derivatives was analyzed by using trypan blue exclusion test and was further confirmed by colorimetric methylthiazolydiphenyl-tetrazolium bromide (MTT) assay as described. Kumar, S., et al., A chromone analog inhibits TNF-alpha induced expression of cell adhesion molecules on human endothelial cells via blocking NF-kappaB activation. Bioorg. Mad. Chem. 2007, 15, 2952-2962. Briefly, Beas-2B cells were treated with chalcone analogs or DMSO alone (0.1%, as vehicle) for 24 h. Four hours before the end of incubation, the medium was removed and 100 μL of MTT (5 mg/mL in serum free medium) was added to each well. The MTT was removed after 4 h, cells were washed with PBS, and 100 μL DMSO was added to each well to dissolve the water-insoluble MTT-formazan crystals. The absorbance was recorded at 570 nm in a plate reader (Molecular Devices, Sunnyvale, Calif.).

c. Generation of Stable Transfectants.

Beas-2B cells overexpressing ARE luciferase reporter plasmid were obtained by transfecting Beas-2B cells with 3 μg of NQO1-ARE reporter plasmid and 0.3 μg of pUB6 empty vector (Invitrogen). Stable transfectants were selected using blasticidin at a concentration of 6 μg/mL. Stable clones were expanded and screened for the expression of ARE luciferase. Micheli, F., et al., A combinatorial approach to [1,5]benzothiazepine derivatives as potential antibacterial agents. J. Comb. Chem. 2001, 3, 224-228.

d. ARE Reporter Assay.

Beas-2B cells stably expressing NQO1-ARE luciferase were seeded onto a 96-well plate at a density of 10,000 cells/well for 16 h before incubation with test compounds. Next day, cells were treated with the indicated concentrations of compound 2b. Cells also were treated with DMSO, which was used as the solvent. The reporter activity was measured after 16 h exposure using the luciferase assay kit (Promega, Madison, Wis.). The level of increase in luciferase activity reflects the degree of Nrf2 activity. Singh, A., et al., Dysfunctional KEAP1-NRF2 interaction in non-small-cell lung cancer. PLoS Mad. 2006, 3, 1866-1876.

e. Mice In Vivo Study.

All experiments in mice were performed in accordance with the standards established by the U.S. Animal Welfare Acts, set forth in NIH guidelines and the Policy and Procedures Manual of the Johns Hopkins University Animal Care and Use Committee. C57BL/6 mice (male, 7 weeks) were maintained on AIN 76A diet (Harlan Tekland, Madison, Wis.) and water ad libitum and housed at a temperature (range, 20-23° C.) under 12 h light/dark cycles. The mice were treated with chalcone analogs (50 mg/kg body weight) or vehicle or sulforaphane as a positive control by gavage. After 24 h treatment, the small intestines were harvested and stored at −80° C. until analysis.

f. RNA Extraction and Gene Expression Analysis.

Total RNA was extracted from cells/tissue using Qiagen RNeasy kit (Qiagen Corporation, Valencia, Calif.) and reverse transcription was performed by using random hexamers and MultiScribe reverse transcriptase according to the manufacturer's recommendations (Applied Biosystems, Foster City, Calif., USA). Quantitative real-time RT-PCR analyses of Nrf2, NQO1, HO1, and GCLM were performed by using Assay-on-Demand primers and probe sets from Applied Biosystems. Assays were performed using the ABI 7000 Taqman system (Applied Biosystems, Foster City, Calif.). β-actin was used for normalization.

3. Statistics

The values are represented as mean±SE and analyzed by student's t-test. Differences were considered significant at P≦0.05.

### Example 2

### 2-trifluoromethyl-2′-methoxychalone Mitigates Mortality in Mice Exposed to Total Body Radiation

Radiation injury can occur from external irradiation, either total body irradiation or partial body irradiation, during radiotherapy, accidental exposures, or nuclear threat due to terrorist or war-time activity. Radiation exposure causes short-term (acute radiation syndrome) or long-term disorders, e.g., lung fibrosis. Clinical components of acute radiation syndrome include hematopoietic, gastrointestinal, and cerebrovascular syndrome that occurs within days to a few weeks.

Hematopoietic syndrome is seen with significant partial-body or whole-body radiation exposures characterized by hypoplasia or aplasia of the bone marrow. These changes result in pancytopenia predisposition to infection, bleeding, and poor wound healing, all of which can contribute to death.

In gastrointestinal syndrome, radiation induces loss of intestinal crypts and breakdown of the mucosal barrier. These changes result in abdominal pain, diarrhea, and nausea and vomiting, and predispose patients to infection.

Cutaneous syndrome includes cutaneous injury from thermal or radiation burns and is characterized by loss of epidermis and dermis. Injuries to the skin might cover only small areas, but can extend deeply into the soft tissue, even reaching underlying muscle and bone.

Mechanism of radiation injury: ROS and electrophiles generated by irradiation are key players in causing acute and chronic pathological injury. ROS induces oxidative damage to biomolecules and causes apoptosis of hematopoietic cells, endothelial cells, and epithelial cells. Depletion of hematopoietic cells results in impaired immune response and predispose patients to secondary infection. Increased death of endothelial cells and epithelial cells results in loss of mucosal barrier and tissue injury. Loss of intestinal or lung mucosal barriers lead to translocation of bacteria into systemic circulation and can cause systemic inflammation and sepsis. On the other hand, tissue injury causes local inflammation leading to tissue remodeling and fibrosis. Taken together, irradiation induces oxidative stress, apoptosis, and inflammation that can lead to multi-organ injury and death. Therapies directed toward blocking ROS-induced deleterious effects can help in mitigating, as well as, treating radiation-induced injury.

Nrf2 is a primary regulator of antioxidant genes. It has been reported that Nrf2 is a primary regulator of a network of cytoprotective genes, including antioxidants in different organs, such as the lung, intestine, liver, and brain, in response to chemical activators or stressors. The antioxidant associated genes regulated by Nrf2 include direct antioxidants (SOD1, heme oxygenase-1 (Hmox1), and NQO1) and genes associated with the glutathione pathway (flutathione peroxidase (Gpx), glutathione reductase, glutamate cysteine ligase (catalytic and modifier subunit), thioredoxin pathway (thioredoxin reductase (Txnrd1), perosiredoxin (Prdx)), as well as NADPH-regenerating enzymes (glucose 6-phophate dehydrogenase (G6PD), phosphogluconate dehydrogenase (Pgd), and maleic enzyme 1 (Me1)) and xenobiotic detoxification enzymes, such as glutathione S-transferase (GST). In addition, Nrf2 regulates several other genes as described in Table 3, which function in a concerted fashion along with antioxidant to attenuate pathological damage caused by reactive oxygen species (ROS), reactive nitrogen species (RNS), and electrophiles generated after radiation exposure.

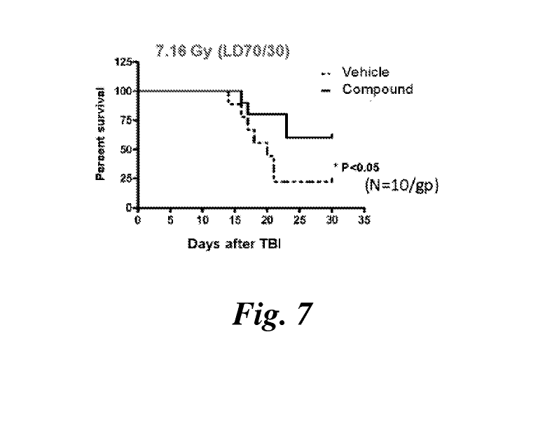

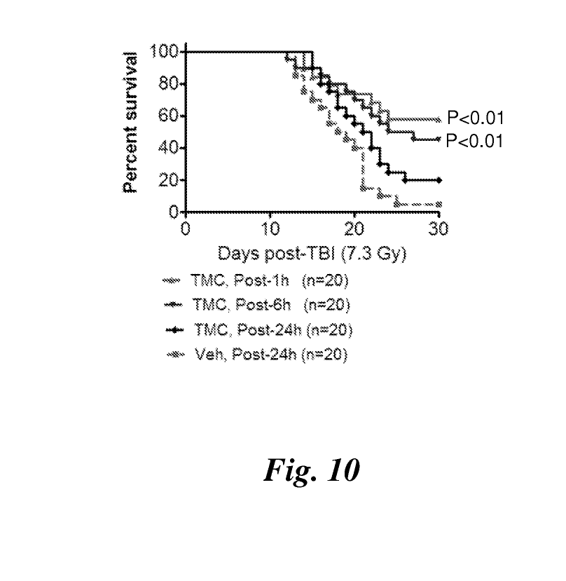

To determine whether 2-trifluoromethyl-2′-methoxychalone, a potent Nrf2 activator, improves survival after lethal irradiation, irradiated mice were treated with 2-trifluoromethyl-2′-methoxychalone (200 μmol/kg body weight) or vehicle (100% PEG) by gavage. Treatment with compound 2b was initiated at one hour and 24 hours after irradiation. Five additional doses of compound 2b or vehicle were given every 48 hours after the first dose. Mice treated with vehicle showed only 25% and 10% survival after exposure to 7.13 Gy and 7.3 Gy. (LD70/30; 70% death in 30 days). See FIG. 7. Additionally, in a separate experiment, it was observed that the median survival increased from approximately 15 days in the vehicle-treated group to 26 days in mice treated with compound 2b 24 hours after irradiation at a higher dose (7.3 Gy, LD100/30; 100% death in 30 days. See FIGS. 10-12.

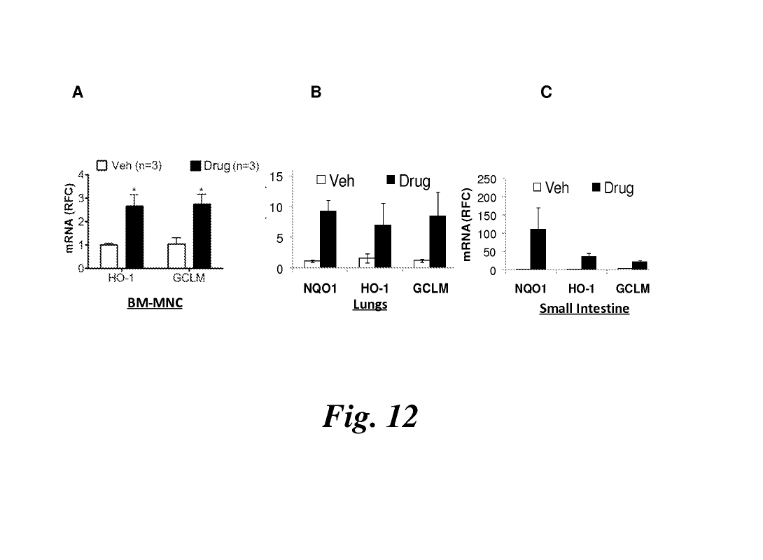

Hematopoietic reconstitution also was evaluated over a period of 30 days in mice treated with the compound 2b 24 hours after 7.3 Gy TBI. CBC analysis showed significant recovery of WBC in irradiated mice treated with the compound 2b. See FIGS. 12A and 12B. Taken together, these data suggest that 2-trifluoromethyl-2′-methoxychalone significantly improves hematopoietic reconstitution and mitigates mortality after radiation exposure.

### Example 3

### Radioprotective and Radiomitigation Efficacy of 2-trifluoromethyl-2′-methoxychalone

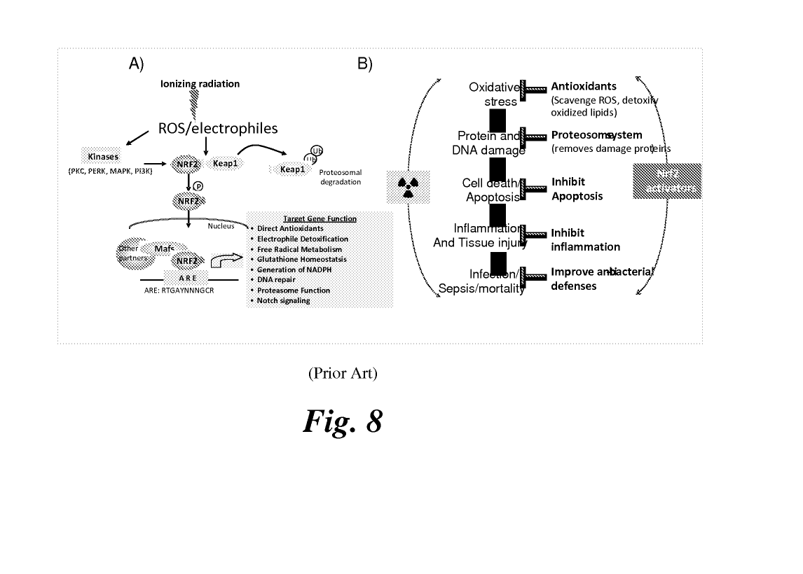

Nrf2 is activated in response to oxidative insult by dissociating from its cytoplasmic anchor, Keap1, and upregulating a transcriptional program that includes genes encoding for antioxidants, DNA repair, proteasome and electrophile detoxification proteins (FIG. 8; Boutten et al., 2011; Kensler et al., 2006). These cytoprotective defense programs counteract oxidative stress, as well as repair and remove cytotoxic oxidative damage byproducts of DNA, protein and lipids. Nrf2 null cells or mice show increased lipid peroxidation byproducts, protein carbonyls, DNA damage and cell death. Pharmacological activation of Nrf2, pre- and post-oxidative insult, significantly decrease protein carbonyls and inhibit cell death. More recently, Nrf2 has been shown to regulate the transcriptional expression of Notch 1, which is essential for tissue repair and hematopoietic stem cell self-renewal. Nrf2 null cells show impaired notch signaling and Nrf2 null mice show impaired tissue regeneration (Malhotra et al., 2010). Activation of Nrf2 augmented tissue regeneration (Malhotra et al., 2010). Because radiation-induced hematopoietic injury mediates cell death and/or impairs regeneration of hematopoietic stem cells, Nrf2 may be a potential drug target for mitigating radiation injuries including, but not limited to, hematopoietic and GI syndrome. Unlike single antioxidant scavengers, small molecules targeting Nrf2 upregulate a broad spectrum of cytoprotective proteins that can be more effective as radio-mitigators and provide whole body protection from radiation-induced injuries. Furthermore, it can be used as a potent radio-protector to inhibit normal tissue injury that occurs during radiotherapy.

To investigate the therapeutic efficacy of compound 2b to mitigate mortality and the hematopoietic syndrome, mice were subjected to total body irradiation (TBI). C57BL/6 (8 wks, males,) were fed chow pellets ad libitum and supplied non-acidic water. Mice were subjected to TBI (0.65 Gy/min) in an AECL Gamma cell 40 irradiator (Atomic Energy, Canada). Compound 2b was dissolved in DMSO: PEG-200 (1:100). Treatment with compound 2b (orally at dose 400 mg/Kg) or vehicle (PEG-200) was initiated 1 h, 6 h, or 24 h after TBI. Five additional doses of compound 2b or vehicle were given every 48 h after the first dose.

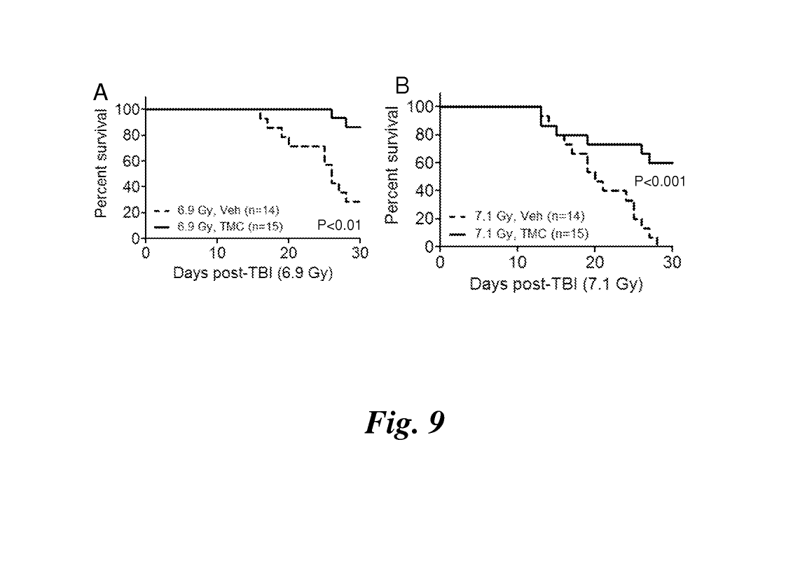

FIG. 9 shows data representing Kaplan-Meier analysis of survival after TBI. Mice were orally administered with compound 2b or vehicle (PEG-200) 24 h after TBI of 6.9 Gy or 7.1 Gy and five additional doses were administered every 48 h. Mortality was monitored every day for 30 days. Mice treated with compound 2b 24 h after TBI at doses 6.9 Gy and 7.1 Gy significantly improved survival to 90% and 60% respectively compared to mice treated with the vehicle (FIG. 9).

To determine if the survival of the mice improved if compound 2b was administered earlier than 24 h after TBI, mice were orally administered with compound 2b or vehicle 1 h, 6 h, or 24 h after TBI (7.3 Gy). Five additional doses of compound 2b or vehicle were given every 48 h after the first dose. Mortality was monitored every day for 30 days. Compound 2b significantly improved the survival of mice treated 1 h and 6 h after TBI to approximately 60% and 50%, respectively, compared to the mice treated with the vehicle (FIG. 10). The percent mortality of mice treated with the vehicle at 1 h or 6 h after radiation was 100% (data not shown). In addition, the survival rate of the mice was better if compound 2b was administered earlier than 24 h after radiation.

### Example 4

### 2-trifluoromethyl-2′-methoxychalone Accelerates Hematopoietic Recovery

To determine if compound 2b was able to accelerate hematopoietic recovery, mice were orally administered compound 2b or vehicle 24 h after total body irradiation with 6.9 Gy (LD30/30). Five additional doses of compound 2b or vehicle were given every 48 h after the first dose. At indicated time periods, a cohort of mice was sacrificed. Peripheral blood was collected by heart puncture at the indicated days and differential blood cell count was analyzed using Hemavet 950FS(P<0.05, n=3 per group). Femur bones were fixed, decalcified sectioned along the sagittal plan and stained with hematoxylin and eosin for analyzing marrow cellularity.

The mice treated with compound 2b showed an early increase in white blood cells (WBC), neutrophils and lymphocytes post-irradiation compared to the mice treated with the vehicle (FIG. 11A). On day 22 post-irradiation, mice treated with compound 2b showed a significant increase in WBC, red blood cells (RBC) and platelets (FIG. 11B). Histopathological analysis by H&E staining of bone marrow isolated from mice treated with compound 2b at day 7 and day 20 showed greater marrow cellularity compared to bone marrow isolated from mice treated with the vehicle (FIG. 11C).

Thus, the presently disclosed data illustrate the therapeutic efficacy of compound 2b to mitigate mortality and hematopoietic syndrome.

### Example 5

### Mechanism of Action of 2-Trifluoromethyl-2′-Methoxychalone

To determine if compound 2b could increase Nrf2 regulated antioxidants in irradiated mice, the pharmacodynamic (PD) markers of Nrf2 activity (NQO1, HO-1 and GCLM) were monitored in the bone marrow mononuclear cells (BM-MC), gut and lungs of irradiated mice (FIG. 12). NQO1, HO-1 and GCLM levels were analyzed in bone marrow (FIG. 12A), lung (FIG. 12B) and small intestine (FIG. 12C) harvested at day 12 from irradiated (6.9Gy) mice treated with vehicle or drug.

Nrf2 regulated antioxidants were significantly elevated in BM-MNC, gut and lung of irradiated mice treated with drug compare to vehicle (FIG. 12). These data demonstrate that compound 2b increases Nrf2 regulated antioxidants.

To determine if compound 2b could increase the levels of bone marrow mononuclear cells and hematopoietic stem cells in irradiated mice, the mice were treated with a single dose of compound 2b 24 h after irradiation (6.9Gy). The total number of BM-MNC and subpopulation of hematopoietic stem cells were analyzed in irradiated mice 24 h after compound 2b treatment.

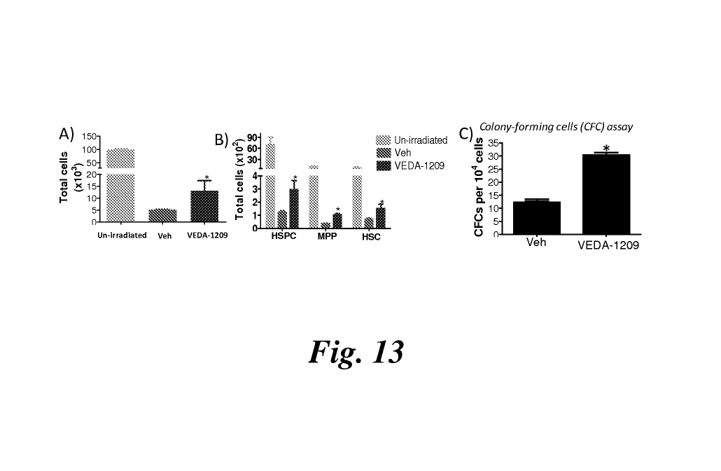

FIG. 13 shows the total number of BM-MNC (Panel A), the frequency of subpopulation of hematopoietic stem cells by FACS analysis (Panel B), and the total number of viable hematopoietic stem cells as assessed by colony forming cells assay (Panel C) (P<0.05, compared to vehicle; n=5). Total BM-MNC per limb (femur and tibia) (FIG. 13A) and total hematopoietic stem progenitor cells (HSPC, ckit+, sca−1+lin−), hematopoietic stem cell (HSC, ckit+, sca1+, lin− and CD150+, CD48−) and multipotent progenitor (MPP, ckit+, sca−1+, lin− and CD150−, CD48−) were significantly higher in mice treated with compound 2b compared to mice treated with the vehicle (FIG. 13B). Colony forming cells assay also demonstrated an increased number of hematopoietic stem cells in mice treated with compound 2b compared to mice treated with the vehicle (FIG. 13C).

The presently disclosed data show that a potential mechanism of action of compound 2b is to enhance the survival of hematopoietic stem cells by increasing the Nrf2 regulated antioxidant defense.

### Example 6

### 2-trifluoromethyl-2′-methoxychalone as a Treatment of Chemotherapy-Induced Neutropenia

Neutropenia, a condition in which neutrophils are at abnormally low levels, occurs with common chemotherapy regimens in 25% to 40% of treatment-naive patients, and its severity depends on the dose intensity of the chemotherapy regimen. Neutropenia and its subsequent infectious complications represent the most common dose-limiting toxicity of cancer chemotherapy. Neutropenia also may lengthen a hospital stay, increase monitoring, diagnostic and treatment costs, and reduce patient quality of life.

To determine whether compound 2b reduces chemotherapy-induced neutropenia, mice were treated with two doses of cyclophosphamide (intraperitoneal injection, day 0 and day 5) to induce reversible neutropenia lasting for 6-7 days. Treatment with compound 2b or vehicle was initiated immediately after the first dose of cyclophosphamide was administered and thereafter every 48 h for the duration of the study. At indicated time periods, a cohort of mice was sacrificed. Peripheral blood was collected and differential blood cell count was analyzed using a Hemavet 950S. Treatment with compound 2b significantly reduced the duration of neutropenia compared to treatment with the vehicle (FIG. 14).

### Example 7

### 2-trifluoromethyl-2′-methoxychalone as a Treatment for Multiple Sclerosis

Multiple sclerosis (MS) is an inflammatory autoimmune disease that affects the central nervous system. It affects at least 350,000 individuals in the United States and 2.5 million people worldwide and the socioeconomic burden of MS is second only to trauma. Clinical signs and symptoms vary among patients, but involve motor, sensory, autonomic and cognitive disabilities. Recent evidence suggests that the pathogenesis of MS is mediated by Th-1 and Th-17 inflammation. Unpublished data suggest that Nrf2 suppresses Th17 inflammation and therefore, small molecule activators of Nrf2 could be potential candidates for the treatment of MS and other Th-17 driven autoimmune inflammatory diseases, such as psoriasis, psoriatic arthritis, rheumatoid arthritis and intestinal bowel disease.

The mouse model for MS, experimental autoimmune encephalomyelitis (EAE), a well characterized model of Th17 cell mediated auto-immune disease, has been extensively utilized for testing the pre-clinical efficacy of drug candidates. EAE is induced by subcutaneous injection with myelin oligodendrocyte (MOG) peptide in complete Freund's adjuvant and interperitoneal injection of pertussis toxin. Induction of EAE occurs within 12-16 days and is scored according to well-established objective criteria. The effects of compound 2b treatment were evaluated in an EAE model. Mice were immunized with MOG33-55 emulsified in complete Freund's adjuvant (CFA).

To assess the prophylactic efficacy, after MOG immunization at day 0, mice (n=5) were treated with compound 2b (400 mg/kg, orally) or vehicle (prophylactic mode) every other day for the duration of the study (FIG. 15A). To assess therapeutic efficacy (n=5), compound 2b or vehicle treatment was initiated at day 9 after MOG immunization and continued thereafter every other day for the duration of the study (FIG. 15B). Both in prophylactic and therapeutic mode, compound 2b significantly delayed the onset and clinical severity of EAE compared to vehicle (FIG. 15).

### Example 8

### 2-trifluoromethyl-2′-methoxychalone as a Treatment for Allergic Asthma

Asthma affects more than 300 million people worldwide, with 250,000 annual deaths (Adcock et al., 2008). In the last 20 years, the prevalence of asthma has doubled, affecting ≧10% of the United States population and is the leading cause of hospitalization among young children (Akinbami and Schoendorf, 2002). Allergic asthma is a complex inflammatory disorder in which typically innocuous allergens trigger a response that is characterized by airway inflammation, intermittent reversible airway obstruction, airways hyperresponsiveness (AHR), excessive mucus production, and elevated levels of IgE and Th2 cytokines. Airborne allergens are inhaled into the lungs and deposited into the alveoli where they are recognized by professional antigen presenting cells (APCs, such as dendritic cells (DCs)), and presented to naïve T cells in the lymph node. Th2 polarized T cells are a major contributor to damage and/or obstruction of airways in response to allergens.

Emerging evidence indicates a critical role for Nrf2 in modulation of inflammation in allergic asthma. Nrf2 deficiency causes a greater expression of Th2 cytokines (IL-4 and IL-13) in splenocytes after ovalbumin (OVA) challenge, indicating Nrf2 may modulate Th2 inflammation. Clinical studies suggest that children with severe asthma have greater oxidative stress and lower concentrations of glutathione in plasma and airway lavage than healthy controls (Fitzpatrick et al., 2011). A recent study suggests that Nrf2 signaling pathway is defective in asthmatics. Dworski, Free Radic. Biol. Med. 2011, Jul. 15; 51(2):516-21; Michaeloudes, Am. J. Respir. Crit. Care. Med. 2011 Oct. 15; 184(8):894-903.

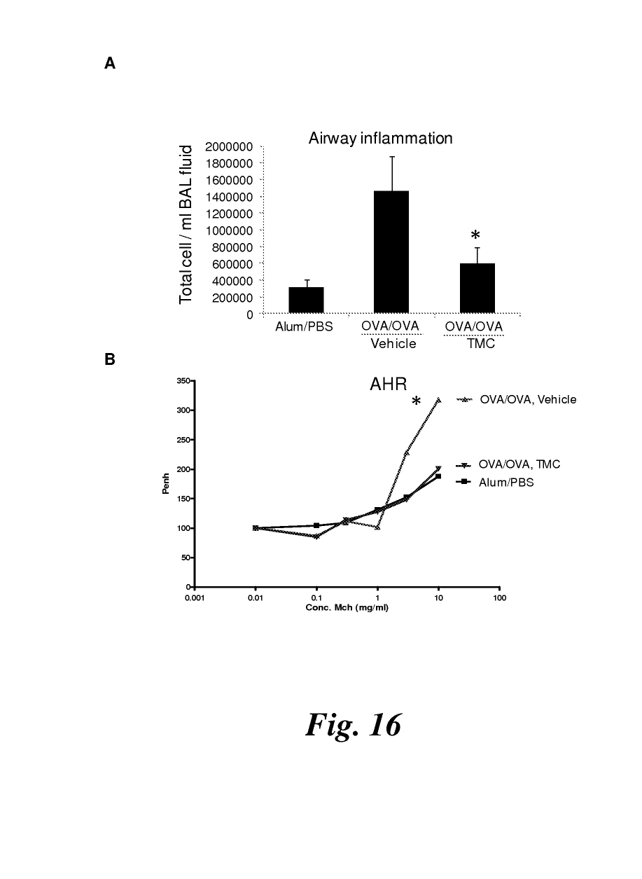

To evaluate whether compound 2b treatment inhibited allergic asthma in a mouse model, a standard mouse model of OVA-induced asthma (Rangasamy et al., 2005) was used. Mice were sensitized by intraperitoneal injection (day 0, day 14), and challenged intranasally, with OVA (day 21 to day 24). Mice were treated with 400 mg/mice of compound 2b (also termed TMC) or vehicle every other day during the sensitization and challenge phase. 24 h after the last dose of OVA challenge, airway inflammation (FIG. 16A) and airway hyperresponsiveness (AHR) (FIG. 16B) were assessed. It was found that compound 2b treatment in a prophylactic mode protected the mice from allergen-induced asthma as assessed by airway inflammation and airway hyper-responsiveness (AHR) (FIG. 16).

### Example 9

### 2-trifluoromethyl-2′-methoxychalone as a Treatment for COPD

COPD is a major worldwide public health problem that is characterized by progressive irreversible airflow limitation (Yoshida and Tuder, 2007). COPD comprises emphysema (alveolar destruction) and airway narrowing caused by chronic bronchitis. COPD significantly impairs quality of life, predisposes to disability, and causes high healthcare costs and early mortality. About 16 million people in the U.S. are affected by COPD, causing at least 125,000 deaths per year and costing $20-30 billion per year (Wise, 2004). COPD is the only leading cause of death that has shown increased mortality in the past 30 years and it is currently the fourth leading cause of death (Barnes, 2007). Although COPD is primarily caused by cigarette smoking, a growing body of evidence suggests other inhaled noxious agents, such as indoor biomass fuel smoke, environmental particles, pathogens (bacteria and virus) as important etiological factors (Hogg and Timens, 2008). The pathogenesis of COPD is mediated by chronic abnormal airway inflammation and oxidative damage that lead to remodeling of lung extracellular matrix, enhancement of mucus secretion, heightened alveolar cell apoptosis versus impaired cell repair and proliferation, and persistence inflammation (Yoshida and Tuder, 2007). COPD is complicated by frequent and recurrent acute exacerbations that are described as the episodes of worsening respiratory symptoms (COPD-related), such as dyspnea, cough, and sputum production that is often followed by subsequent clinical deterioration (decline in FEV1) (Veeramachaneni and Sethi, 2006; Wedzicha and Seemungal, 2007). The frequency of these exacerbations shows correlation with a decline in the lung function, which is the cause for substantial morbidity and mortality of patients with COPD (Donaldson et al., 2002; Kanner et al., 2001; Anzueto et al., 2007).

COPD is associated with poor lung innate immune defenses, particularly impaired phagocytic ability of alveolar macrophages. A growing body of evidence suggests that exacerbations of COPD are largely caused by bacterial infections, such as Nontypeable Haemophilus influenzae (NTHI), Streptococcus pneumoniae, Moraxella catarrhalis, Pseudomonas aeruginosa (PA), and Staphylococcus aureus (Wedzicha and Seemungal, 2007). Currently, there are no effective therapies to limit COPD exacerbations. Treatment with antibiotics is discouraged due to increasing rates of antibiotic resistance and adverse effects associated with prolonged use. Treatment with corticosteroids shows limited efficacy due to the development of corticosteroid resistance in patients with COPD.

It has recently been demonstrated that enhancing Nrf2 improves the phagocytic ability of macrophages, as well as inhibits LPS-induced inflammation in macrophages isolated from patients with COPD or mice exposed to cigarette smoke.

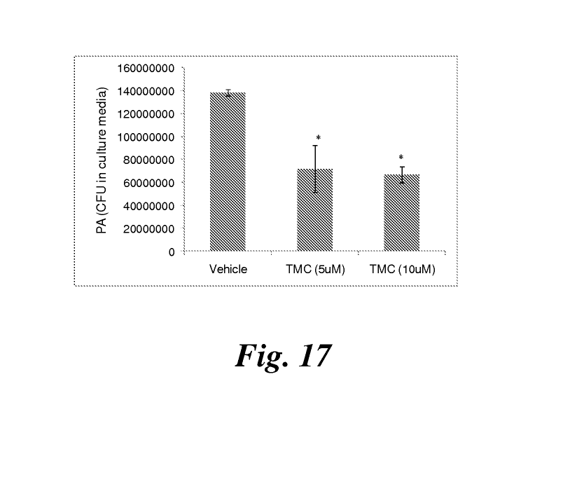

To evaluate whether compound 2b increased bacterial clearance by macrophages, normal mice were treated with compound 2b (5 μM or 10 μM) for 16-20 h, the peritoneal macrophages were isolated from the mice, and the peritoneal macrophages were incubated with Pseudomonas aeruginosa (PA). After 4 h, the bacterial burden was analyzed in cell-free media by plating on to blood agar. This study showed that compound 2b significantly decreased bacteria burden in culture media as indicated by colony forming units (CFU) when compared to the vehicle treatment (FIG. 17).

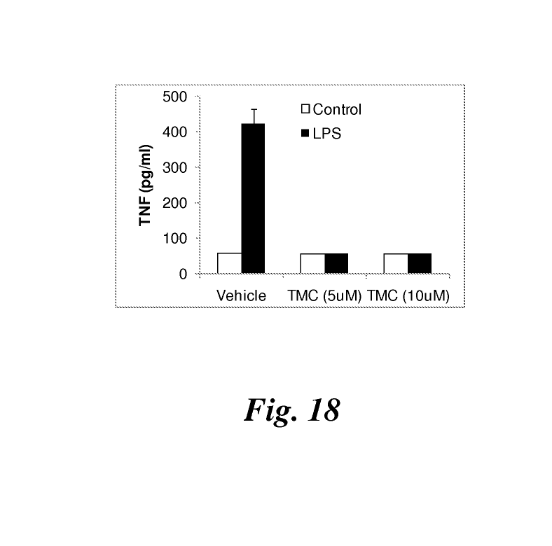

To determine if compound 2b inhibited LPS-induced inflammation, normal mice were treated with compound 2b (5 μM or 10 μM) for 16-20 h, the peritoneal macrophages were isolated from the mice, and the peritoneal macrophages were incubated with LPS (100 ng/mL). After 4 h, TNF-α secretion in cell-free media by macrophages was analyzed by ELISA. Results from this study showed a significant decrease in LPS-induced TNF-α secretion by macrophages when the mice were treated with compound 2b when compared to the treatment with the vehicle (FIG. 18).

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.
Kensler, T. W.; Wakabayash, N.; Biswal, S. Cell survival responses to environmental stresses via the Keap1-Nrf2-ARE pathway. Annu. Rev. Pharmacool. Toxicol. 2007, 47, 89-116.
Rangasamy, T.; Guo, J.; Mitzner, W. A.; Roman, J.; Singh, A.; Fryer, A. D.; Yamamoto, M.; Kensler, T. W.; Tuder, R. M.; Georas, S, N.; Biswal, S. Disruption of Nrf2 enhances susceptibility to severe airway inflammation and asthma in mice. J. Exp. Med. 2005, 202, 47-59.
Sussan, T. E.; Rangasamy, T.; Blake, D. J.; Malhotra, D.; EI-Haddad, H.; Bedja, D.; Yates, M. S.; Kombairaju, P.; Yamamoto, M.; Liby, K. T.; Sporn, M. B.; Gabrielson, K. l.; Champion, H. C.; Tuder, R. M.; Kensler, T. W.; Biswal, S. Targeting Nrf2 with the triterpenoid CDDO-imidazolide attenuates cigarette smoke-induced emphysema and cardiac dysfunction in mice. Proc. Natl. Acad. Sci. U.S.A. 2009, 106, 250-255.
Thimmulappa, R. K.; Mai, K H.; Srisuma, S.; Kensler, T. W.; Yamamato, M.; Biswal, S. Identification of Nrf2-regulated genes induced by the chemopreventive agent sulforaphane by oligonucleotide microarray. Cancer Res. 2002, 62, 5196-5203.
Misra, V; Lee, H.; Singh, A; Huang, K; Thimmulappa, R. K; Mitzner, W.; Biswal, S.; Tankersley, C. G. Global expression profiles from C57BL/6J and DBA/2J mouse lungs to determine aging-related genes. Physiological Genomics. 2007, 31, 429-440.
Surh, Y. J.; Kundu, J. K.; Na, H. K. Nrf2 as a Master Redox Switch in Turning on the Cellular Signaling Involved in the Induction of Cytoprotective Genes by Some Chemopreventive Phytochemicals. Planta Med. 2008, 74, 1526-1539.
Singh, A; Boldin-Adamsky, S.; Thimmulappa, R. K; Rath, S. K.; Ashush, H; Coulter, J.; Blackford, A; Goodman, S, N.; Bunz, F.; Watson, W. H.; Gabrielson, E.; Feinstein, E.; Biswal, S. RNAi-mediated silencing of nuclear factor erythroid-2-related factor 2 gene expression in non-small cell lung cancer inhibits tumor growth and increases efficacy of chemotherapy. Cancer Res. 2008, 68, 7975-7984.
Niture, S. K.; Jain, A. K; Jaiswal, A. K. Antioxidant-induced modification of INrf2 cysteine 151 and PKC-delta-mediated phosphorylation of Nrf2 serine 40 are both required for stabilization and nuclear translocation of Nrf2 and increased drug resistance. J. Cell Sci. 2009, 122, 4452-4464.
Cheng, S. E.; Lee, I. T.; Lin, C. C.; Kou, Y. R.; Yang, C. M. Cigarette smoke particle-phase extract induces HO-1 expression in human tracheal smooth muscle cells: role of the c-Src/NADPH oxidase/MAPK/Nrf2 signaling pathway. Free Radical Bioi. Med. 2010, 48, 1410-1422.
Nioi, P.; McMahon, M.; Itoh, K.; Yamamoto, M.; Hayes, J. D. Identification of a novel Nrf2-regulated antioxidant response element (ARE) in the mouse NAD(P)H: quinone oxidoreductase 1 gene: reassessment of the ARE consensus sequence. Biochem. J. 2003, 374, 337-348.
Chartoumpekis, D.; Ziros, P. G.; Psyrogiannis, A; Kyriazopoulou, V.; Papavassiliou, A. G.; Habeos, I. G. Simvastatin lowers reactive oxygen species level by Nrf2 activation via PI3K/Akt pathway. Biochem. Biophys. Res. Commun. 2010, 396, 463-466.
Cullinan, S. B.; Diehl, J. A. PERK-dependent activation of Nrf2 contributes to redox homeostasis and cell survival following endoplasmic reticulum stress. J. Biol. Chem. 2004, 279, 20108-20117.
Giudice, A; Arra, C.; Turco, M. C. Review of Molecular Mechanisms Involved in the Activation of the Nrf2-ARE Signaling Pathway by Chemopreventive Agents. Transcription Factors: Methods and Protocols. 2010, 37-74.
Wan, J. K; Diaz-Sanchez, D. Antioxidant enzyme induction: A new protective approach against the adverse effects of diesel exhaust particles. Inhal. Toxicol. 2007, 19, 177-182.
Singh, A; Rangasamy, T.; Thimmulappa, R. K.; Lee, H.; Osburn, W. O.; Brigelius-Flohe, R; Kensler, T. W.; Yamamoto, M.; Biswal, S. Glutathione peroxidase 2, the major cigarette smoke-inducible isoform of GPX in lungs, is regulated by Nrf2. Am. J. Respir. Cell Mol. Biol. 2006, 35, 639-650.
Lin, H. J.; Zhou, H. Y.; Dai, A. H.; Huang, H. F.; Lin, J. H.; Frankl, H. O.; Lee, E. R; Haile, R. W. Glutathione transferase GSTT1, broccoli, and prevalence of colorectal adenomas. Pharmacogenetics. 2002, 12, 175-179.
Kirsch, M.; De Groot, H. NAD(P)H, a directly operating antioxidant? FASEB J. 2001, 15, 1569-1574.
Lee, J. S.; Surh, Y. J. Nrf2 as a novel molecular target for chemoprevention. Cancer Lett. 2005, 224, 171-184.
Nguyen, T.; Sharratt, P. J.; Pickett, C. B. Regulatory mechanisms controlling gene expression mediated by the antioxidant response element. Annu. Rev. Pharmacool. Toxicol. 2003, 43, 233-260.
Dinkova-Kostova, A. T.; Holtzclaw, W. D.; Cole, R. N.; Itoh, K.; Wakabayashi, N.; Katoh, Y.; Yamamoto, M.; Talalay, P. Direct evidence that sulfhydryl groups of Keap1 are the sensors regulating induction of phase 2 enzymes that protect against carcinogens and oxidants. Proc. Natl. Acad. Sci. U.S.A. 2002, 99, 11908-11913.
Yu, R; Lei, W.; Mandlekar, S.; Weber, M. J.; Der, C. J.; Wu, J.; Kong, A. N. T. Role of a mitogen-activated protein kinase pathway in the induction of phase II detoxifying enzymes by chemicals. J. Bioi. Chem. 1999, 274, 27545-27552.
McMahon, M.; Itoh, K; Yamamoto, M.; Hayes, J. D. Keap1-dependent proteasomal degradation of transcription factor Nrf2 contributes to the negative regulation of antioxidant response element-driven gene expression. J. Biol. Chem. 2003, 278, 21592-21600.
Kwak, M. K.; Itoh, K.; Yamamoto, M.; Kensler, T. W. Enhanced expression of the transcription factor Nrf2 by cancer chemopreventive agents: Role of antioxidant response element-like sequences in the nrf2 promoter. Mol. Cell. Biol. 2002, 22, 2883-2892.
Kwak, M. K.; Wakabayashi, N.; Itoh, K.; Motohashi, H.; Yamamoto, M.; Kensler, T. W. Modulation of gene expression by cancer chemopreventive dithiolethiones through the Keap1-Nrf2 pathway—Identification of novel gene clusters for cell survival. J. Biol. Chem. 2003, 278, 8135-8145.
Kwak, M. K.; Egner, P. A.; Dolan, P. M.; Ramos-Gomez, M.; Groopman, J. D.; Itoh, K.; Yamamoto, M.; Kensler, T. W. Role of phase 2 enzyme induction in chemoprotection by dithiolethiones. Mutat. Res. 2001, 480-1, 305-315.
Balogun, E.; Hogue, M.; Gong, P. F.; Killeen, E.; Green, C. J.; Foresti, R.; Alam, J.; Motterlini, R. Curcumin activates the haem oxygenase-1 gene via regulation of Nrf2 and the antioxidant-responsive element. Biochem. J. 2003, 371, 887-895.
Go, M. L.; Wu, X.; Uu, X. L. Chalcones: An update on cytotoxic and chemoprotective properties. Curr. Med. Chem. 2005, 12, 483-499.
Dimmock, J. R; Elias, D. W.; Beazely, M. A; Kandepu, N. M. Bioactivities of chalcones. Curr. Med. Chem. 1999, 6, 1125-1149.
Xia, Y.; Yang, Z. Y.; Xia, P.; Bastow, K. F.; Nakanishi, Y.; Lee, K. H. Antitumor agents. Part 202: Novel 2′-amino chalcones: Design, synthesis and biological evaluation. Bioorg. Med. Chem. Lett. 2000, 10, 699-701.
Bois, F.; Beney, C.; Boumendjel, A; Mariotte, A. M.; Conseil, G.; Di Pietro, A. Halogenated chalcones with high-affinity binding to P-glycoprotein: Potential modulators of multidrug resistance. J. Med. Chem. 1998, 41, 4161-4164.
Liu, M.; Wilairat, P.; Go, M. L. Antimalarial alkoxylated and hydroxylated chalcones: Structure-activity relationship analysis. J. Med. Chem. 2001, 44, 4443-4452.
Dominguez, J. N.; Charris, J. E.; Lobo, G.; de Dominguez, N. G.; Moreno, M. M.; Riggione, F.; Sanchez, E.; Olson, J.; Rosenthal, P. J. Synthesis of quinolinyl chalcones and evaluation of their antimalarial activity. Eur. J. Mad. Chem. 2001, 36, 555-560.
Hsieh, H. K.; Lee, T. H.; Wang. J. P.; Wang, J. J.; Lin, C. N. Synthesis and anti-inflammatory effect of chalcones and related compounds. Pharm. Res. 1998, 15, 39-46.
Hsieh, H. K.; Tsao, L. T.; Wang, J. P.; Lin, C. N. Synthesis and anti-inflammatory effect of chalcones. J. Pharm. Pharmacol. 2000, 52, 163-171.
Herencia, F.; Ferrandiz, M. L; Ubeda, A.; Dominguez, J. N.; Charris, J. E.; Lobo, G. M.; Alcaraz, M. J. Synthesis and anti-inflammatory activity of chalcone derivatives. Bioorg. Med. Chem. Lett. 1998, 8, 1169-1174.
Lin, Y. M.; Zhou, Y. S.; Flavin, M. T.; Zhou, L. M.; Nie, W. G.; Chen, F. C. Chalcones and flavonoids as anti-tuberculosis agents. Biorg. Med. Chem. 2002, 10, 2795-2802.
Rojas, J.; Paya, M.; Dominguez, J. N.; Ferrandiz, M. L. The synthesis and effect of fluorinated chalcone derivatives on nitric oxide production. Bioorg. Med. Chem. Lett. 2002, 12, 1951-1954.
Herencia, F.; Ferrandiz, M. L.; Ubeda, A; Guillen, I.; Dominguez, J. N.; Charris, J. E; Lobo, G. M.; Alcaraz, M. J. 4-dimethylamino-3′,4′-dimethoxychalcone downregulates iNOS expression and exerts anti-inflammatory effects. Free Radical Biol. Med. 2001, 30, 43-50.
Ducki, S.; Forrest, R.; Hadfield, J. A.; Kendall, A; Lawrence, N. J.; McGown, A. T.; Rennison, D. Potent antimitotic and cell growth inhibitory properties of substituted chalcones. Bioorg. Med. Chem. Lett. 1998, 8, 1051-1056.
Maria, K; Dimitra, H. L.; Maria, G. Synthesis and Anti-Inflammatory Activity of Chalcones and Related Mannich Bases. Med. Chem. 2008, 4, 586-596.
Viana, G. S. B.; Bandeira, M. A. M.; Matos, F. J. A. Analgesic and antiinflammatory affects of chalcones isolated from Myracrodruon urundeuva Allemao. Phytomedicine. 2003, 10, 189-195.
Cheng, J. H.; Hung, C. F.; Yang, S. C.; Wang, J. P.; Won, S. J.; Lin, C. N. Synthesis and cytotoxic, anti-inflammatory, and anti-oxidant activities of 2′,5′-dialkoxylchalcones as cancer chemopreventive agents. Biorg. Med. Chem. 2008, 44, 16, 7270-7276.
Heidari, M. R.; Foroumadi, A; Amirabadi, A; Samzadeh-Kermani, A; Azimzadeh, B. S.; Eskandarizadeh, A. Evaluation of Anti-inflammatory and Analgesic Activity of a Novel Rigid 3,4-Dihydroxy Chalcone in Mice. Natural Compounds and Their Role in Apoptotic Cell Signaling Pathways. 2009, 1171, 399-406.
Cheenpracha, S.; Karalai, C.; Ponglimanont, C.; Subhadhirasakul, S.; Tewtrakul, S. Anti-HIV-1 protease activity of compounds from Boesenbergia pandurata. Biorg. Med. Chem. 2006, 14, 1710-1714.
Svetaz, L.; Tapia, A; Lopez, S, N.; Furlan, R l. E.; Petenatti, E.; Pioli, R.; Schmeda-Hirschmann, G.; Zacchino, S. A. Antifungal chalcones and new caffeic acid esters from Zuccagnia punctata acting against soybean infecting fungi. J. Agric. Food. Chem. 2004, 52, 3297-3300.
Saavedra, M. J.; Borges, A.; Dias, C.; Aires, A.; Bennett, R. N.; Rosa, E. S.; Simoes, M. Antimicrobial activity of phenolics and glucosinolate hydrolysis products and their synergy with streptomycin against pathogenic bacteria. Med. Chem. 2010, 6, 174-83.
Aponte, J. C.; Verastegui, M.; Malaga, E,; Zimic, M.; Quiliano, M.; Vaisberg, A. J.; Gilman, R. H.; Hammond, G. B. Synthesis, cytotoxicity, and anti-Trypanosoma cruzi activity of new chalcones. J. Med. Chem. 2008, 51, 6230-6234.
Lahtchev, K. L.; Batovska, D. I.; Parushev, S. P.; Ubiyvovk, V. M.; Sibirny, A. A. Antifungal activity of chalcones: A mechanistic study using various yeast strains. Eur. J. Med. Chem. 2008. 43, 2220-2228.
Batovska, D. I.; Todorova, I. T. Trends in utilization of the pharmacological potential of chalcones. Curr Clin Pharmacol. b2010, 5, 1-29.
Torigoe, T,; Arisawa, M.; Itoh, S.; Fujiu, M.; Maruyama, H. B. Anti-mutagenic chalcones-antagonizing the mutagenicity of benzo (A) pyrene on salmonella-typhimurium. Biochem. Biophys. Res. Commun. 1983, 112, 833-842.
Lee, S. A; Ryu, H. W.; Kim, Y. M.; Choi, S.; Lee, M. J.; Kwak, T. K.; Kim, H. J.; Cho, M.; Park, K. H.; Lee, J. W. Blockade of Four-Transmembrane L6 Family Member 5 (TM4SF5)-Mediated Tumorigenicity in Hepatocytes by a Synthetic Chalcone Derivative. Hepatology. 2009, 49, 1316-1325.
Shibata, S. Anti-tumerigenic chalcones. Stem Cells. 1994, 12, 44-52.
Devincenzo, R.; Scambia, G.; Panici, P. B.; Ranelletti, F. O.; Bonanno, G.; Ercoli, A; Dellemonache, F.; Ferrari, F.; Piantelli, M.; Mancuso, S. Effect of synthetic and naturally-occurring chalcones on ovarian-cancer cell-growth-structure-activity-relationships. Anti-Cancer Drug Des. 1995, 10, 481-490.
Wei, B. L.; Teng, C. H.; Wang, J. P.; Won, S. J.; Lin, C. N. Synthetic 2′,5′-dimethoxychalcones as G(2)/M arrest-mediated apoptosis-inducing agents and inhibitors of nitric oxide production in rat macrophages. Eur. J. Med. Chem. 2007, 42, 660-668.
Takahashi, T.; Takasuka, N.; Iigo, M.; Baba, M.; Nishino, H.; Tsuda, H.; Okuyama, T. Isoliquiritigenin, a flavonoid from licorice, reduces prostaglandin E-2 and nitric oxide, causes apoptosis, and suppresses aberrant crypt foci development. Cancer Sci. 2004, 95, 448-453.
Lee, J. H.; Jung, H. S.; Giang, P. M.; Jin, X.; Lee, S.; Son, P. T.; Lee, D.; Hong, Y. S.; Lee, K; Lee, J. J. Blockade of nuclear factor-kappa B signaling pathway and anti-inflammatory activity of cardamomin, a chalcone analog from Alpinia conchigera. J. Pharmacol. Exp. Ther. 2006, 316, 271-278.
Ban, H. S.; Suzuki, K; Lim, S. S.; Jung, S. H.; Lee, S.; Ji, J.; Lee, H. S.; Lee, Y. S.; Shin, K H.; Ohuchi, K Inhibition of lipopolysaccharide-induced expression of inducible nitric oxide synthase and tumor necrosis factor-alpha by 2′-hydroxychalcone derivatives in RAW 264.7 cells. Biochem. Pharmacal. 2004, 67, 1549-1557.
Liu, Y. C.; Hsieh, C. W.; Wu, C. C.; Wung, B. S. Chalcone inhibits the activation of NF-kappa Band STAT3 in endothelial cells via endogenous electrophile. Life Sci. 2007, 80, 1420-1430.
Foresti, R.; Hogue, M.; Monti, D.; Green, C. J.; Motterlini, R. Differential activation of heme oxygenese-1 by chalcones and rosolic acid in endothelial cells. J. Pharmacol. Exp. Ther. 2005, 312, 686-693.
Wu, C. C.; Hsieh, C. W.; Lai, P. H.; Lin, J. B.; Liu, Y. C.; Wung, B. S. Upregulation of endothelial heme oxygenase-1 expression through the activation of the JNK pathway by sublethal concentrations of acrolein. Taxicol. Appl. Pharmacol. 2006, 214, 244-252.
Shibata, T.; Yamada, T.; Ishii, T.; Kumazawa, S.; Osawa, T.; Nakamura, H.; Masutani, H.; Yodol, J.; Uchida, K. Thioredoxin as a molecular target of cyclopentenone prostaglandins. Free Radical Biol. Med. 2003, 35, 384.
Heiss, E.; Herhaus, C.; Klimo, K.; Bartsch, H.; Gerhauser, C. Nuclear factor kappa B is a molecular target for sulforaphane-mediated anti-inflammatory mechanisms. J. Biol. Chem. 2001, 276, 32008-32015.
Micheli, F.; Degiorgis, F.; Feriani, A; Paio, A; Pozzan, A; Zarantonello, P.; Seneci, P. A combinatorial approach to [1,5]benzothiazepine derivatives as potential antibacterial agents. J. Comb. Chem. 2001, 3, 224-228.
Bu, X. Y.; Zhao, L. Y.; Li. Y. L. A facile synthesis of 6-C-prenylflavanones. Synthesis-Stuttgart. 1997, 1246-1248.
Sinisterra, J. V.; Garciaraso, A; Cabello, J. A; Marinas, J. M. An improved procedure for the Claisen-Schmidt reaction. Synthesis-Stuttgart. 1984, 502-504.
Alcantara, A. R.; Marinas, J. M.; Sinisterra, J. V. Synthesis of 2′-hydroxychalcones and related-compounds in interfacial solid-liquid conditions. Tetrahedron Lett. 1987, 28, 1515-1518.
Climent, M. J.; Corma, A.; Iborra, S.; Velty, A. Activated hydrotalcites as catalysts for the synthesis of chalcones of pharmaceutical interest. J. Catal. 2004, 221, 474-482.
Daskiewicz, J. B.; Comte, G.; Barron, D.; Di Pietro, A.; Thomasson, F. Organolithium mediated synthesis of prenylchalcones as potential inhibitors of chemoresistance. Tetrahedron Lett. 1999, 40, 7095-7098.
Sebti, S.; Solhy, A; Smahi, A.; Kossir, A.; Oumimoun, H. Dramatic activity enhancement of natural phosphate catalyst by lithium nitrate. An efficient synthesis of chalcones. Catal. Commun. 2002, 3, 335-339.
Calloway, N. O.; Green, L. D. Reactions in the presence of metallic halides 1 beta-unsaturated ketone formation as a side reaction in Friedel-Crafts acylations. J. Am. Chem. Soc. 1937, 59, 809-811.
Breslow, D. S.; Hauser, C. R. Condensations. XI. Condensations of certain active hydrogen compounds effected by boron trifluoride and aluminum chloride. J. Am. Chem. Soc. 1940, 62, 2385-2388.
Szell, T.; Sohar, I. New nitrochalcones. Can. J. Chem. 1969, 47, 1254-1258.
Irie, K; Watanabe, K. Aldol condensations with metal(II) complex catalysts. Bull. Chem. Soc. Jpn. 1980, 53, 1366-1371.
Nakano, T.; Irifune, S.; Umana, S.; Inada, A.; Ishii, Y.; Ogawa, M. Cross-condensation reactions of cycloalkanones with aldehydes and primary alcohols under the influence of zirconocene complexes. J. Org. Chem. 1987, 52, 2239-2244.
Iranpoor, N.; Kazemi, F. RuCl₃ catalyses aldol condensations of aldehydes and ketones. Tetrahedron. 1998, 54, 9475-9480.
Eddarir, S.; Cotelle, N.; Bakkour, Y.; Rolando, C. An efficient synthesis of chalcones based on the Suzuki reaction. Tetrahedron Lett. 2003, 44, 5359-5363.
Bhagat, S.; Sharma, R; Sawant, D. M.; Sharma, L.; Chakraborti, A. K. LiOH.H₂O as a novel dual activation catalyst for highly efficient and easy synthesis of 1,3-diaryl-2-propenones by Claisen-Schmidt condensation under mild conditions. J. Mol. Catal. A-Chem. 2006, 244, 20-24.
Osburn, W. O.; Yates, M. S.; Dolan, P. D.; Chen, S.; Liby, K. T,; Sporn, M. S.; Taguchi, K.; Yamamoto, M.; Kensler, T. W. Genetic or pharmacologic amplification of Nrf2 signaling inhibits acute inflammatory liver injury in mice. Toxicol. Sci. 2008, 1 04, 218-227.
Bloom, D.; Dhakshinamoorthy, S.; Jaiswal, A. K. Site-directed mutagenesis of cysteine to serine in the DNA binding region of Nrf2 decreases its capacity to upregulate antioxidant response element-mediated expression and antioxidant induction of NAD(P)H:quinone oxidoreductase1 gene. Oncogene. 2002, 21, 2191-2200.
Nguyen, T.; Nioi, P.; Pickett, C. B. The Nrf2-antioxidant response element signaling pathway and its activation by oxidative stress. J. Bioi. Chem. 2009, 284, 13291-13295.
McMahon, M.; Lamont, D. J.; Beattie, K. A; Hayes, J. D. Keap1 perceives stress via three sensors for the endogenous signaling molecules nitric oxide, zinc, and alkenals. Proc. Natl. Acad. Sci. U.S.A. 2010, 107, 18838-18843.
Kumar, S.; Singh, B. K.; Pandey, A K.; Kumar, A; Sharma, S. K; Raj, H. G.; Prasad, A. K.; Van der Eycken, E.; Parmar, V. S.; Ghosh, B. A chromone analog inhibits TNF-alpha induced expression of cell adhesion molecules on human endothelial cells via blocking NF-kappaB activation. Bioorg. Mad. Chem. 2007, 15, 2952-2962.
Singh, A; Misra, V.; Thimmulappa, R. K.; Lee, H.; Ames, S.; Hogue, M. O.; Herman, J. G.; Baylin, S. R; Sidransky, D.; Brock, M. V.; Gabrielson, E.; Biswal, Dysfunctional KEAP1-NRF2 interaction in non-small-cell lung cancer. PLoS Mad. 2006, 3, 1866-1876.
Boutten A, Goven D, Artaud-Macari E, Boczkowski J, Bonay M. NRF2 targeting: a promising therapeutic strategy in chronic obstructive pulmonary disease. Trends Mol Med 2011; 17(7) 363-371.
Kensler T W, Wakabayashi N, Biswal S. Cell Survival Responses to Environmental Stresses Via the Keap1-Nrf2-ARE Pathway. Annu Rev Pharmacol Toxicol 2006; 47:89-116.
Malhotra D, Portales, Casamar E, Singh A, Srivastava S, Arenillas D, Happel C, Shyr C, Wakabayashi N, Kensler T W, Wasserman W W, Biswal S. Global mapping of binding sites for Nrf2 identifies novel targets in cell survival response through ChIP-Seq profiling and network analysis. Nucleic Acids Res 2010; 38(17):5718-34.
Adcock I M, Caramori G, Chung K F. New targets for drug development in asthma. Lancet 2008; 372:1073-1087.
Akinbami L J, Schoendorf K C. Trends in childhood asthma: prevalence, health care utilization, and mortality. Pediatrics 2002; 110:315-322.
Fitzpatrick A M, Stephenson S T, Hadley G R, Burwell L, Penugonda M, Simon D M, Hansen J, Jones D P, Brown L A. Thiol redox disturbances in children with severe asthma are associated with posttranslational modification of the transcription factor nuclear factor (erythroid-derived 2)-like 2. J Allergy Clin Immunol 2011; 127:1604-1611.
Rangasamy T, Guo J, Mitzner W A, Roman J, Singh A, Fryer A D, Yamamoto M, Kensler T W, Tuder R M, Georas S N, Biswal S. Disruption of Nrf2 enhances susceptibility to severe airway inflammation and asthma in mice. J Exp Med 2005; 202:47-59.
Yoshida T, Tuder R M. Pathobiology of cigarette smoke-induced chronic obstructive pulmonary disease. Physiol Rev 2007; 87:1047-1082.
Wise R: The Epidemiology of Chronic Obstructive Pulmonary Disease. In: Lung volume reduction Surgery for Emphysema, vol. 184: Marcel Dekkerer; 2004.
Barnes P J. Chronic obstructive pulmonary disease: a growing but neglected global epidemic. PLoS Med 2007; 4:e112.
Hogg J C, Timens W. The Pathology of Chronic Obstructive Pulmonary Disease. Annu Rev Pathol 2008.
Veeramachaneni S B, Sethi S. Pathogenesis of bacterial exacerbations of COPD. COPD 2006; 3:109-115.
Wedzicha J A, Seemungal T A. COPD exacerbations: defining their cause and prevention. Lancet 2007; 370:786-796.
Donaldson G C, Seemungal T A, Bhowmik A, Wedzicha J A. Relationship between exacerbation frequency and lung function decline in chronic obstructive pulmonary disease. Thorax 2002; 57:847-852.
Kanner R E, Anthonisen N R, Connett J E. Lower respiratory illnesses promote FEV(1) decline in current smokers but not ex-smokers with mild chronic obstructive pulmonary disease: results from the lung health study. Am J Respir Crit Care Med 2001; 164:358-364.
Anzueto A, Sethi S, Martinez F J. Exacerbations of chronic obstructive pulmonary disease. Proc Am Thorac Soc 2007; 4:554-564.
Dworski, Free Radic. Biol. Med. 2011, Jul. 15; 51(2):516-21.
Michaeloudes, Am. J. Respir. Crit. Care. Med. 2011 Oct. 15; 184(8):894-903.
Merchant, A A, Singh, A, Matsui, W, Biswal, S, The redox-sensitive transcription factor Nrf2 regulates murine hematopoietic stem cell survival independently of ROS levels, Blood 2011; 118(25):6572-6579.

Although the foregoing subject matter has been described in some detail by way of illustration and example for purposes of clarity of understanding, it will be understood by those skilled in the art that certain changes and modifications can be practiced within the scope of the appended claims.

1. A compound of Formula (Ia): 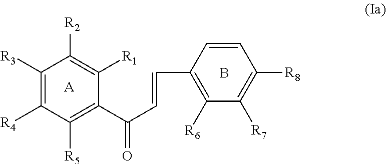
wherein:
R₁, R₂, R₃, R₄, and R₅ are each independently selected from the group consisting of H and alkoxyl, provided that at least one of R₁, R₂, R₃, R₄, and R₅ is alkoxyl;
R₆ and R₇ are each independently selected from the group consisting of H, CF₃, and NO₂, provided that at least one of R₆ and R₇ is CF₃ or NO₂;
R₈ is H;
under the further provision that if R₆ or R₇ is CF₃, then R₁ and R₃, or R₂ and R₃, or R₁ and R₄ cannot both be alkoxyl;
and pharmaceutically acceptable salts thereof.

2. The compound of claim 1, wherein the compound of Formula (Ia) is selected from the group consisting of: 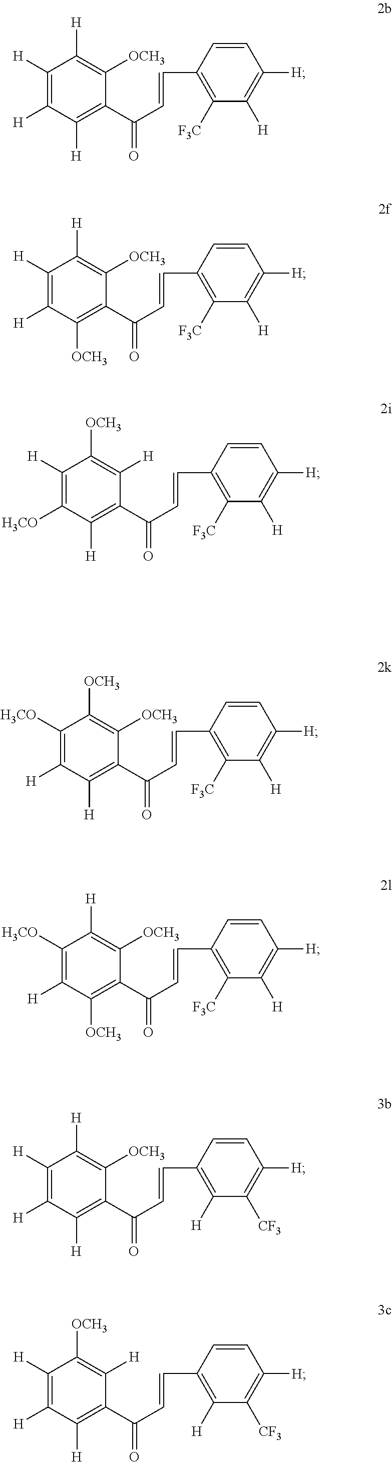 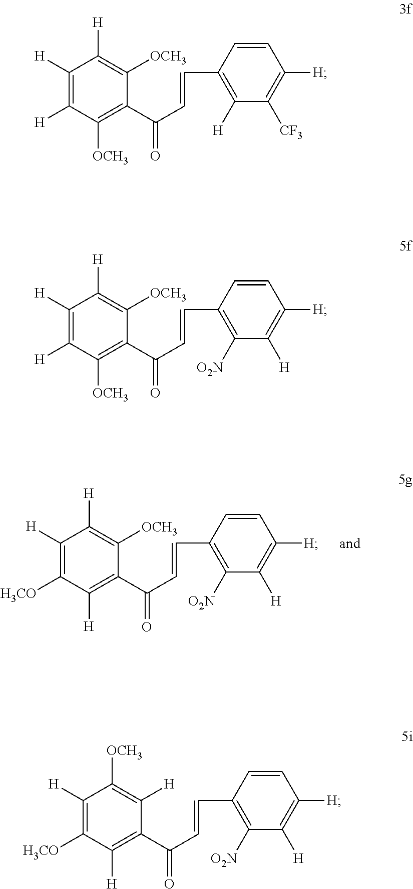
pharmaceutically acceptable salts thereof.

3. The compound of claim 2, wherein the compound of Formula (Ia) is selected from the group consisting of: 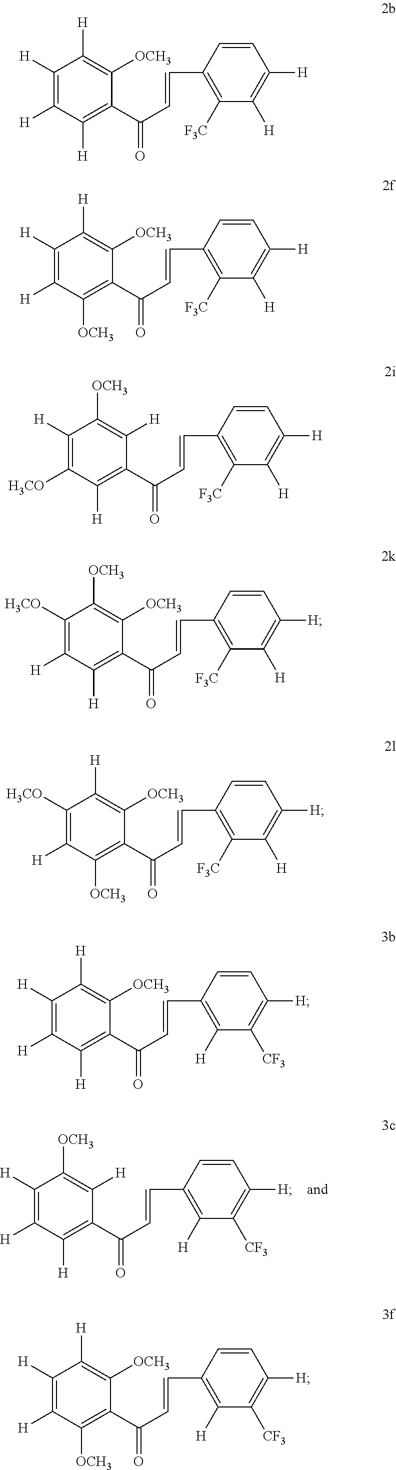
and pharmaceutically acceptable salts thereof.

4. The compound of claim 3, wherein the compound of Formula (Ia) is selected from the group consisting of: 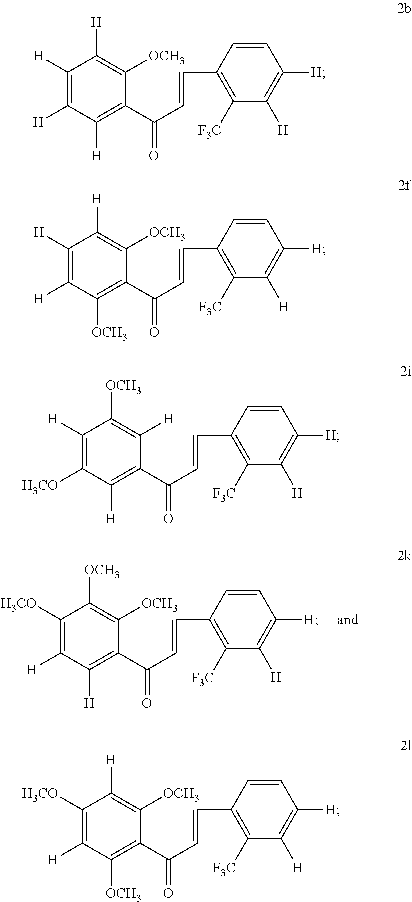
and pharmaceutically acceptable salts thereof.

5. The compound of claim 4, wherein the compound of Formula (Ia) is: 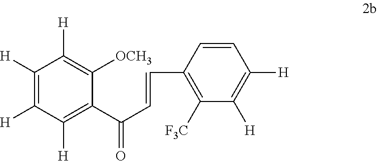 [SMILES: COc1ccccc1C(=O)/C=C/c1ccccc1C(F)(F)F]
and pharmaceutically acceptable salts thereof.

6. A pharmaceutical composition comprising a compound of Formula (Ia) and a pharmaceutically acceptable carrier.

7. The pharmaceutical composition of claim 6, further comprising one or more agents selected from the group consisting of a corticosteroid, an antibiotic, and combinations thereof.

8. The pharmaceutical composition of claim 7, wherein the corticosteroid is selected from the group consisting of dexamethasone, flunisolide, fluticasone propionate, triamcinolone acetonide, beclomethasone dipropionate, budesonide, prednisone, prednisolone, and methylprednisolone.

9. The pharmaceutical composition of claim 7, wherein the pharmaceutical composition is formulated for inhalation or oral administration.

10. A method for treating or preventing a disease, disorder or condition associated with an Nrf2-regulated pathway, the method comprising administering a compound of Formula (Ib) to the subject in an amount effective to increase an Nrf2 biological activity or Nrf2 expression, thereby treating or preventing the disease, disorder, or condition: 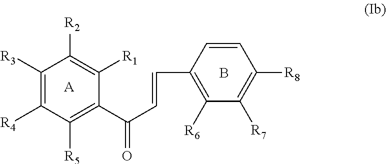
wherein:
R₁, R₂, R₃, R₄, and R₅ are each independently selected from the group consisting of H and alkoxyl, provided that at least one of R₁, R₂, R₃, R₄, and R₅ is alkoxyl;
R₆, R₇, and R₈ are each independently selected from the group consisting of H, CF₃, and NO₂, provided that at least one of R₆, R₇, and R₈ is CF₃ or NO₂;
and pharmaceutically acceptable salts thereof.

11. The method of claim 10, wherein the compound of Formula (Ib) is selected from the group consisting of: 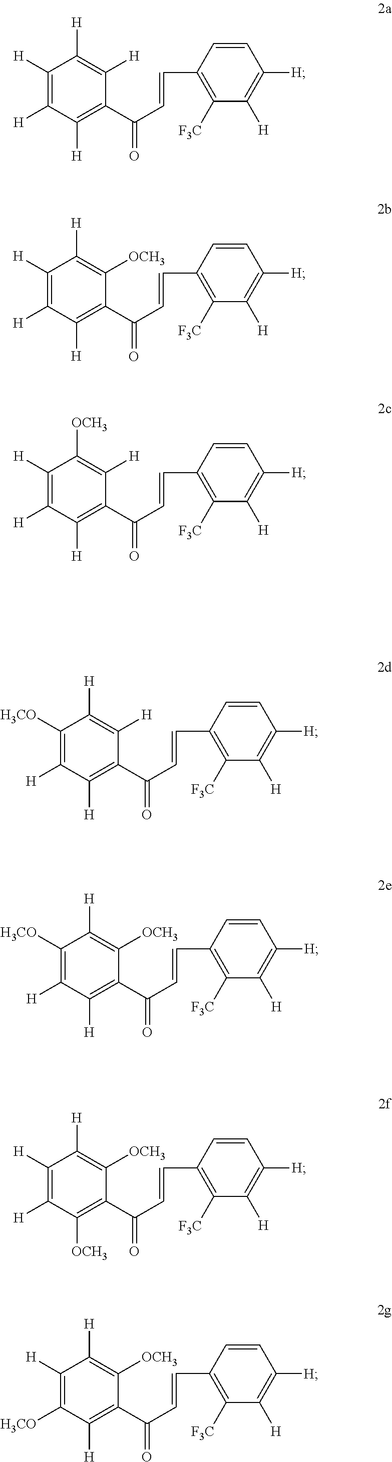 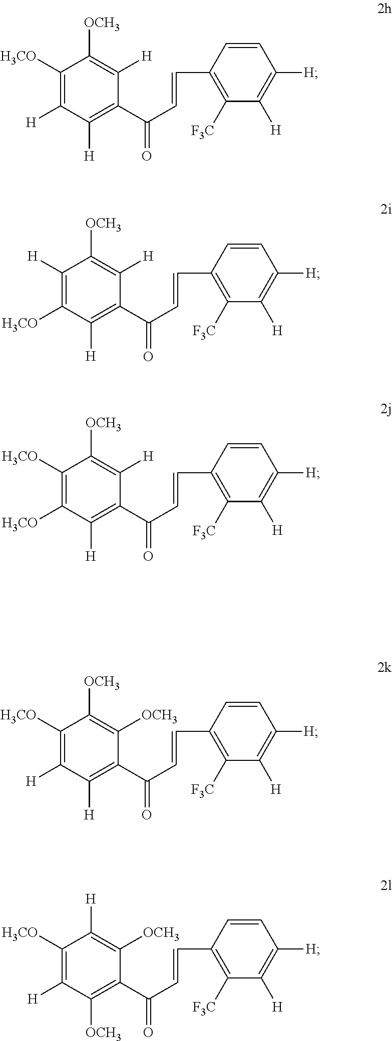
and pharmaceutically acceptable salts thereof.

12. The method of claim 10, wherein the compound of Formula (Ib) is selected from the group consisting of: 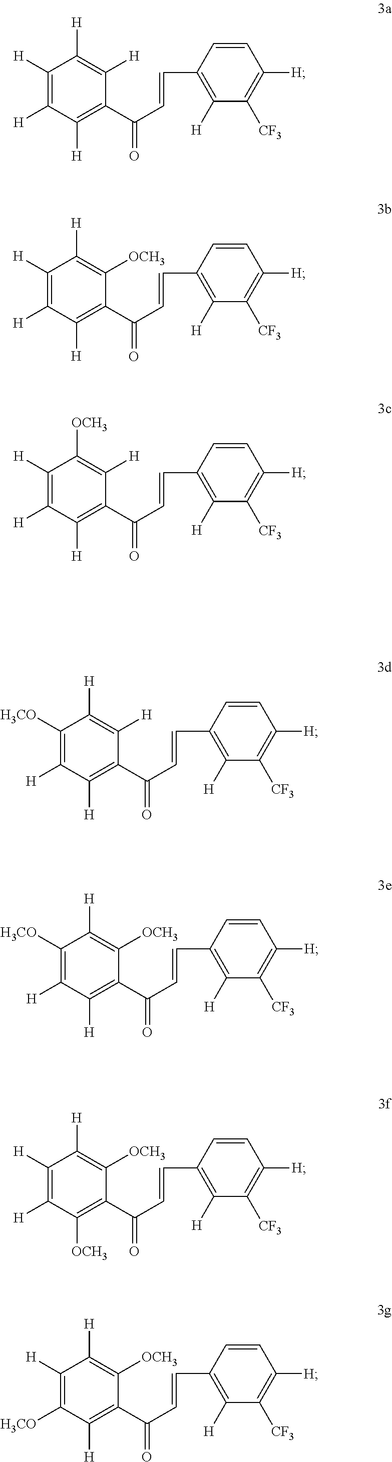 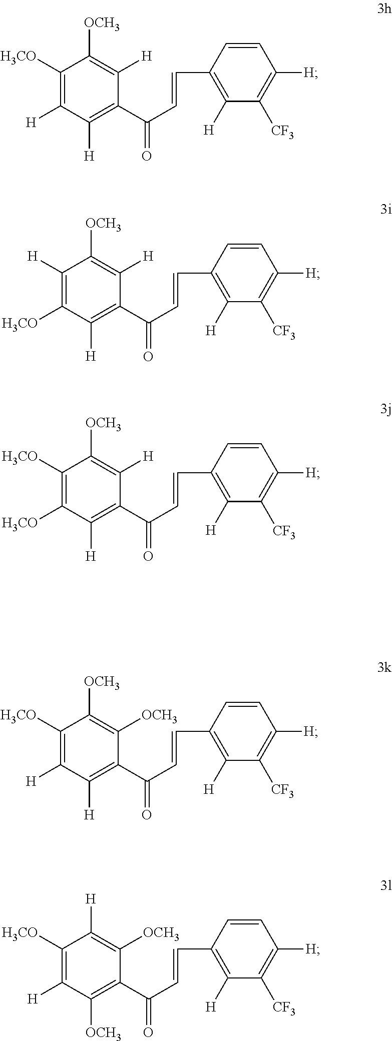
and pharmaceutically acceptable salts thereof.

13. The method of claim 10, wherein the compound of Formula (Ib) is selected from the group consisting of: 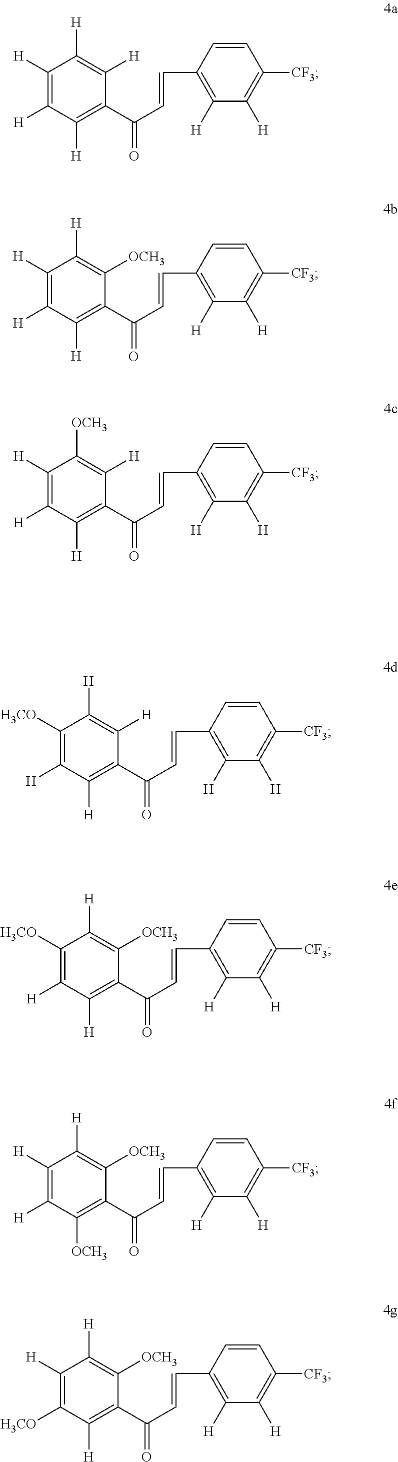 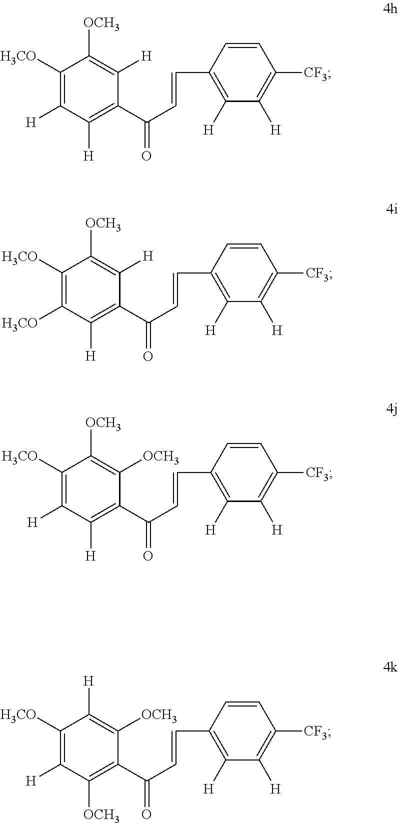 [SMILES: COc1ccc(C(=O)/C=C/c2ccc(C(F)(F)F)cc2)cc1OC ; COc1cc(C(=O)/C=C/c2ccc(C(F)(F)F)cc2)cc(OC)c1OC ; COc1ccc(C(=O)/C=C/c2ccc(C(F)(F)F)cc2)c(OC)c1OC ; COc1cc(OC)c(C(=O)/C=C/c2ccc(C(F)(F)F)cc2)c(OC)c1]
and pharmaceutically acceptable salts thereof.

14. The method of claim 11, wherein the compound of Formula (Ib) is: 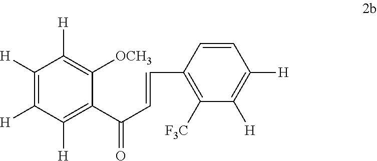 [SMILES: COc1ccccc1C(=O)/C=C/c1ccccc1C(F)(F)F]
and pharmaceutically acceptable salts thereof.

15. The method of claim 10, wherein the disease, disorder, or condition is an autoimmune disease.

16. The method of claim 15, wherein the autoimmune disease is selected from the group consisting of acute graft-versus host disease, autoimmune inner ear disease, inflammatory bowel disease, rheumatoid arthritis, psoriasis, psoriatic arthritis, multiple sclerosis, scleroderma, lupus, ankylosing spondylitis, neutropenia, and uveitis.

17. The method of claim 10, wherein the disease, disorder, or condition is related to a comorbidity associated with diabetes.

18. The method of claim 17, wherein the disease, disorder, or condition related to a comorbidity associated with diabetes is selected from the group consisting of retinopathy and nephropathy.

19. The method of claim 10, wherein the disease, disorder, or condition is related to improving an outcome for bone marrow transplant for leukemia and related cancers, a bone marrow deficiency, an inborn error of metabolism, and an immune disorder.

20. The method of claim 10, wherein the disease, disorder, or condition is related to oxidative stress.

21. The method of claim 20, wherein the disease, disorder, or condition related to oxidative stress is selected from the group consisting of a pulmonary inflammatory condition, interstitial pulmonary fibrosis, asthma, chronic obstructive pulmonary disease (COPD), acute respiratory distress syndrome (ARDS), emphysema, sepsis, septic shock, meningitis, encephalitis, hemorrhage, ischemic injury, cerebral ischemia, heart ischemia, a cognitive deficit, and a neurodegenerative disorder.

22. The method of claim 20, wherein the method reduces subepithelial fibrosis, mucus metaplasia, or a structural alteration associated with airway remodeling.

23. The method of claim 21, wherein the neurodegenerative disease is selected from the group consisting of Alzheimer's disease (AD), Creutzfeldt-Jakob disease, Huntington's disease, Lewy body disease, Pick's disease, Parkinson's disease, amyotrophic lateral sclerosis (ALS), multiple sclerosis (MS), neurofibromatosis, and a cognitive deficit.

24. The method of claim 21, wherein the method prevents or reduces cell death following an ischemic injury.

25. The method of 24, wherein the method reduces cell death in a neural tissue of the subject.

26. The method of claim 10, wherein the method restores a corticosteroid responsiveness in the subject.

27. The method of claim 26, wherein the subject has or is at risk of developing a disease, disorder, or condition selected from the group consisting of chronic obstructive pulmonary disease (COPD), asthma, severe asthma, acute graft-versus host disease, autoimmune inner ear disease, inflammatory bowel disease, and rheumatoid arthritis.

28. The method of claim 26, further comprising administering a corticosteroid to the subject in combination with the compound of Formula (Ib).

29. The method of claim 28, wherein the corticosteroid is selected from the group consisting of dexamethasone, flunisolide, fluticasone propionate, triamcinolone acetonide, beclomethasone dipropionate, budesonide, prednisone, prednisolone, and methylprednisolone.

30. The method of claim 10, wherein the disease, disorder, or condition comprises a respiratory infection.

31. The method of claim 30, wherein the subject has or is at risk of developing a disease, disorder, or condition selected from the group consisting of an acute respiratory infection, chronic bronchitis, cystic fibrosis, and an immunodeficiency syndrome.

32. The method of claim 30, wherein the subject is or was a smoker, has emphysema, or has COPD.

33. The method of claim 30, wherein the respiratory infection is associated with an infectious agent selected from the group consisting of Pseudomonas aeruginosa, nontypeable Haemophilus influenzae, Moraxella catarrhalis, streptococcus pneumonia, staphylococcus aureus, Rhinovirus, coronovirus, influenza A and B, parainfluenza, Adenovirus, and Respiratory syncytial virus.

34. The method of claim 30, further comprising treating or preventing bacterial colonization in a tissue or organ of a subject.

35. The method of claim 34, wherein the tissue is a mucous membrane.

36. The method of claim 34, wherein the organ is a lung.

37. The method of claim 34, further comprising administering an antibiotic in combination with the compound of Formula (Ib).

38. The method of claim 30, wherein the administration of a compound of Formula (Ib) increases a bacterial clearance by macrophages.

39. The method of claim 10, wherein the method comprises treating or preventing a radiation injury or a chemotherapy injury in the subject.

40. The method of claim 39, wherein the method treats or prevents a disease, disorder, or condition selected from the group consisting of hematopoietic syndrome, gastrointestinal syndrome, cerebrovascular syndrome, cerebrospinal injury, pulmonary effects, sepsis, renal failure, pneumonitis, mucositis, enteritis, fibrosis, skin injuries, neutropenia, and an effect on a soft tissue.

41. The method of claim 40, wherein the method treats or prevents a symptom of hematopoietic syndrome selected from the group consisting of hypoplasia or aplasia of the bone marrow, pancytopenia, predisposition to infection, bleeding, and poor wound healing.

42. The method of claim 40, wherein the method treats or prevents a symptom of gastrointestinal syndrome selected from the group consisting of a loss of intestinal crypts, a breakdown of the mucosal barrier, abdominal pain, diarrhea, nausea, and vomiting.

43. The method of claim 39, wherein the method treats or prevents a cutaneous injury from a radiation burn.

44. The method of claim 43, wherein the cutaneous injury is selected from the group consisting of loss of epidermis, loss of dermis, loss of muscle, and loss of bone.

45. The method of claim 39, wherein the method prevents lung fibrosis or esophageal damage associated with radiotherapy.

46. The method of claim 39, wherein the method treats or prevents an inflammation.

47. The method of claim 39, wherein the radiation injury is associated with radiation exposure arising from one or more radiation exposure events selected from the group consisting of radiotherapy, accidental radiation exposure, and nuclear attack.

48. The method of claim 39, wherein the method prevents cell death or damage of a cell selected from the group consisting of a pulmonary cell, a endothelial cell, a pulmonary endothelial cell, a smooth muscle cell, an epithelial cell, and an alveolar cell.

49. The method of claim 39, wherein the compound of Formula (Ib) is administered before, during, or after radiation injury.

50. The method of claim 49, wherein the compound of Formula (Ib) is administered within a time period of about one hour to about 12 hours after exposure of the subject to radiation.

51. The method of claim 49, wherein the compound of Formula (Ib) is administered before exposure of the subject to radiation.

52. The method of claim 10, wherein the disease, disorder, or condition is neutropenia.

53. The method of claim 52, wherein the neutropenia is caused by a condition selected from the group consisting of chemotherapy, autoimmunity, and a congenital neutropenic disorder.

54. The method of claim 10, wherein the method increases Nrf2 transcription or translation.

55. The method of claim 10, wherein the compound of Formula (Ib) increases a Nrf2 biological activity selected from the group consisting of binding to an antioxidant-response element (ARE), nuclear accumulation, and the transcriptional induction of a target gene.

56. The method of claim 55, wherein the Nrf2 target gene is selected from the group consisting of MARCO, HO-1, NQO1, GCLm, GST α1, TrₓR₅ Pxr 1, GSR₅ G6PDH, γGCLm, GCLc, G6PD, GST α3, GST p2, SOD2, SOD3, and GSR.

57. The method of claim 10, wherein the compound of Formula (Ib) reduces Keap1 inhibition of Nrf2.

58. The method of claim 57, wherein the compound of Formula (Ib) disrupts Keap1 binding to Nrf2.

59. A kit for treating or preventing a radiation injury, the kit comprising a therapeutically effect amount of compound of Formula (Ia) and written instructions for use of the kit.

60. A device for dispersing one or more particles comprising a compound of Formula (Ia) in an amount effective to increase a Nrf2 biological activity or Nrf2 expression and delivering a dose of the particles to lung tissue of a subject.

61. The device of claim 60, wherein the device is selected from the group consisting of a nebulizer, a metered dose inhaler, and a dry powder inhaler.
